(19) United States
(12) Patent Application Publication
(10) Pub. No.: US 2024/0226309 A1
(43) Pub. Date: Jul. 11, 2024

(54) HEAD AND NECK CANCER COMBINATION THERAPY COMPRISING AN IL-2 CONJUGATE AND CETUXIMAB

(71) Applicant: Synthorx, Inc., La Jolla, CA (US)

(72) Inventors: Giovanni ABBADESSA, Winchester, MA (US); Carolina E. CAFFARO, San Diego, CA (US); Brigitte DEMERS, Paris (FR); Joseph LEVEQUE, San Diego, CA (US); Wan-Ju MENG, Cambridge, MA (US); Marcos MILLA, Carlsbad, CA (US); Jerod PTACIN, San Diego, CA (US)

(73) Assignee: Synthorx, Inc., La Jolla, CA (US)

(21) Appl. No.: 18/524,157

(22) Filed: Nov. 30, 2023

Related U.S. Application Data
(63) Continuation of application No. PCT/US2022/031974, filed on Jun. 2, 2022.
(60) Provisional application No. 63/196,448, filed on Jun. 3, 2021.
(60) Provisional application No. 63/214,634, filed on Jun. 24, 2021.
(60) Provisional application No. 63/253,892, filed on Oct. 8, 2021.
(60) Provisional application No. 63/257,921, filed on Oct. 20, 2021.

(51) Int. Cl. A61K 47/64; A61K 31/138; A61K 31/165; A61K 31/4178; A61K 39/00; A61P 35/02; C07K 16/22

(52) U.S. Cl. CPC A61K 47/642; A61K 31/138; A61K 31/165; A61K 31/4178; A61P 35/02; C07K 16/22; A61K 2039/505; A61K 2039/545

(57) ABSTRACT

Disclosed herein are methods for treating head and neck squamous cell carcinoma (HNSCC) in a subject in need thereof, comprising administering IL-2 conjugates in combination with cetuximab.

### CROSS-REFERENCE TO RELATED APPLICATIONS

This application is a continuation of International Application No. PCT/US2022/031974, filed on Jun. 2, 2022, which claims priority to U.S. Provisional Application No. 63/196,448, filed on Jun. 3, 2021, U.S. Provisional Application No. 63/214,634, filed on Jun. 24, 2021, U.S. Provisional Application No. 63/253,892, filed on Oct. 8, 2021, and U.S. Provisional Application No. 63/257,921, filed on Oct. 20, 2021.

### SEQUENCE LISTING

This application contains a Sequence listing which has been submitted electronically in XML format and is hereby incorporated by reference in its entirety. Said XML copy, created on Nov. 17, 2023, is named “01183-0155-00US_ST26.xml” and is 3,547 bytes in size.

### BACKGROUND OF THE DISCLOSURE

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+T and B lymphocytes, monocytes, macrophages, endothelial cells, and other tissue residents.

In some instances, interleukin 2 (IL-2) signaling is used to modulate T cell responses and subsequently for treatment of a cancer. Accordingly, in one aspect, provided herein are methods of treating head and neck squamous cell carcinoma (HNSCC) in a subject comprising administering an IL-2 conjugate in combination with one or more additional agents.

### SUMMARY OF THE DISCLOSURE

Described herein are methods of treating head and neck squamous cell carcinoma (HNSCC) in a subject in need thereof, comprising administering to the subject an IL-2 conjugate in combination with one or more additional agents, wherein the IL-2 conjugate comprises the amino acid sequence of SEQ ID NO: 1 having an unnatural amino acid residue described herein at position 64, e.g., the amino acid sequence of SEQ ID NO: 2.

Exemplary embodiments include the following.

Embodiment 1. A method of treating head and neck squamous cell carcinoma (HNSCC) in a subject in need thereof, comprising administering to the subject (a) an IL-2 conjugate and (b) cetuximab, wherein:
the HNSCC is recurrent and/or metastatic HNSCC; and
the IL-2 conjugate comprises the amino acid sequence of SEQ ID NO: 1 wherein the amino acid at position P64 is replaced by the structure of Formula (I):

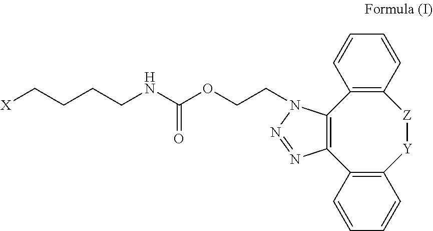

wherein:
Z is CH₂ and Y is

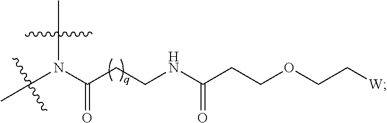

Y is CH₂ and Z is

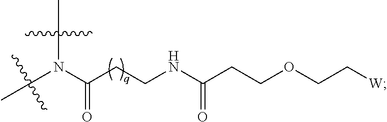

Z is CH₂ and Y is

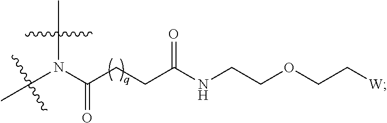

or
Y is CH₂ and Z is

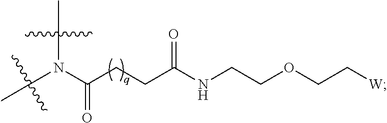

W is a PEG group having an average molecular weight of about 25 kDa-35 kDa;
q is 1, 2, or 3;
X is an L-amino acid having the structure:

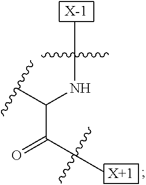

X−1 indicates the point of attachment to the preceding amino acid residue; and
X+1 indicates the point of attachment to the following amino acid residue.

Embodiment 2. A method of treating head and neck squamous cell carcinoma (HNSCC) in a subject in need thereof, comprising:
selecting a subject having HNSCC, wherein the subject is selected at least in part on the basis of the subject having recurrent and/or metastatic HNSCC; and
administering to the subject (a) an IL-2 conjugate, and (b) cetuximab, wherein:
the IL-2 conjugate comprises the amino acid sequence of SEQ ID NO: 1 wherein the amino acid at position P64 is replaced by the structure of Formula (I):

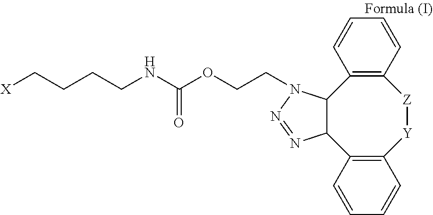

wherein:
Z is CH₂ and Y is

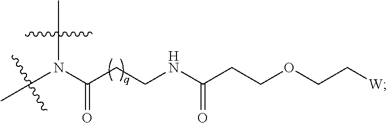

Y is CH₂ and Z is

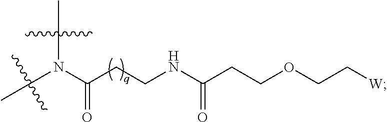

Z is CH₂ and Y is

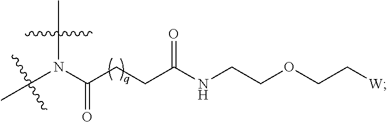

or
Y is CH₂ and Z is

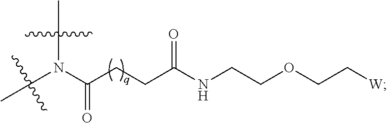

W is a PEG group having an average molecular weight of about 25 kDa-35 kDa;
q is 1, 2, or 3;
X is an L-amino acid having the structure:

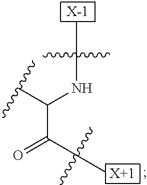

X−1 indicates the point of attachment to the preceding amino acid residue; and
X+1 indicates the point of attachment to the following amino acid residue.

Embodiment 3. A method of treating head and neck squamous cell carcinoma (HNSCC) in a subject in need thereof, comprising administering to the subject (a) from 8 μg/kg to 32 μg/kg IL-2 as an IL-2 conjugate and (b) cetuximab, wherein:
the IL-2 conjugate comprises the amino acid sequence of SEQ ID NO: 1 wherein the amino acid at position P64 is replaced by the structure of Formula (I):

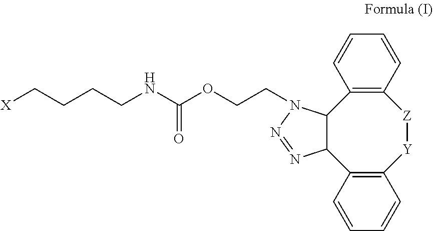

wherein:
Z is CH₂ and Y is

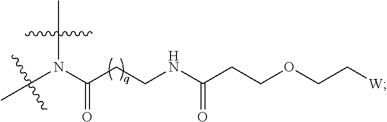

Y is CH₂ and Z is

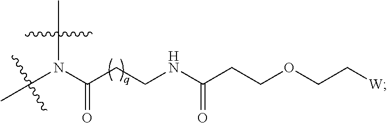

Z is CH₂ and Y is

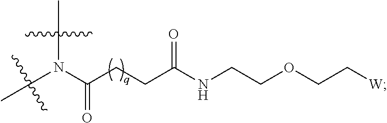

or
Y is CH₂ and Z is

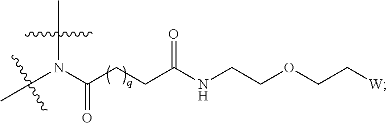

W is a PEG group having an average molecular weight of about 25 kDa-35 kDa;
q is 1, 2, or 3;
X is an L-amino acid having the structure:

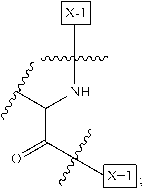

X−1 indicates the point of attachment to the preceding amino acid residue; and
X+1 indicates the point of attachment to the following amino acid residue.

Embodiment 4. The method of any one of embodiments 1-3, wherein the subject was not previously treated with cetuximab.

Embodiment 5. The method of any one of embodiments 1-4, wherein the subject has platinum-refractory HNSCC.

Embodiment 6. The method of any one of embodiments 1-5, wherein the subject was previously treated for HNSCC and the previous treatment for HNSCC comprised failure of no more than two regimens.

Embodiment 7. The method of any one of embodiments 1-6, wherein the subject has platinum-refractory HNSCC and the subject's previous treatment for HNSCC comprised failure of one regimen.

Embodiment 8. The method of any one of embodiments 1-6, wherein the subject has platinum-refractory HNSCC and the subject's previous treatment for HNSCC comprised failure of two regimens.

Embodiment 9. The method of any one of embodiments 1-8, comprising administering to the subject about 8 μg/kg to 32 μg/kg of the IL-2 conjugate.

Embodiment 10. The method of any one of embodiments 1-9, comprising administering to the subject about 8 μg/kg of the IL-2 conjugate.

Embodiment 11. The method of any one of embodiments 1-9, comprising administering to the subject about 16 μg/kg of the IL-2 conjugate.

Embodiment 12. The method of any one of embodiments 1-9, comprising administering to the subject about 24 μg/kg of the IL-2 conjugate.

Embodiment 13. The method of any one of embodiments 1-9, comprising administering to the subject about 32 μg/kg of the IL-2 conjugate.

Embodiment 14. The method of any one of embodiments 1-13, wherein in the IL-2 conjugate the PEG group has an average molecular weight of about 30 kDa.

Embodiment 15. The method of any one of embodiments 1-14, wherein in the IL-2 conjugate Z is CH₂ and Y is

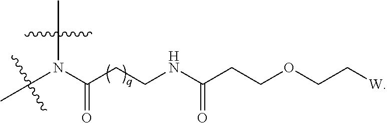

Embodiment 16. The method of any one of embodiments 1-14, wherein in the IL-2 conjugate Y is CH₂ and Z is

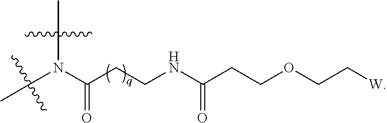

Embodiment 17. The method of any one of embodiments 1-14, wherein in the IL-2 conjugate Z is CH₂ and Y is

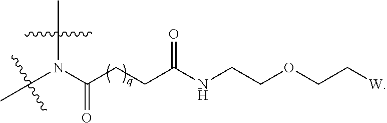

Embodiment 18. The method of any one of embodiments 1-14, wherein in the IL-2 conjugate Y is CH₂ and Z is

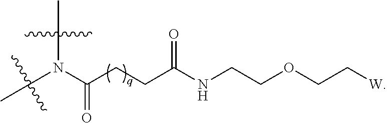

Embodiment 19. The method of any one of embodiments 1-14, wherein the structure of Formula (I) has the structure of Formula (IV) or Formula (V), or is a mixture of Formula (IV) and Formula (V):

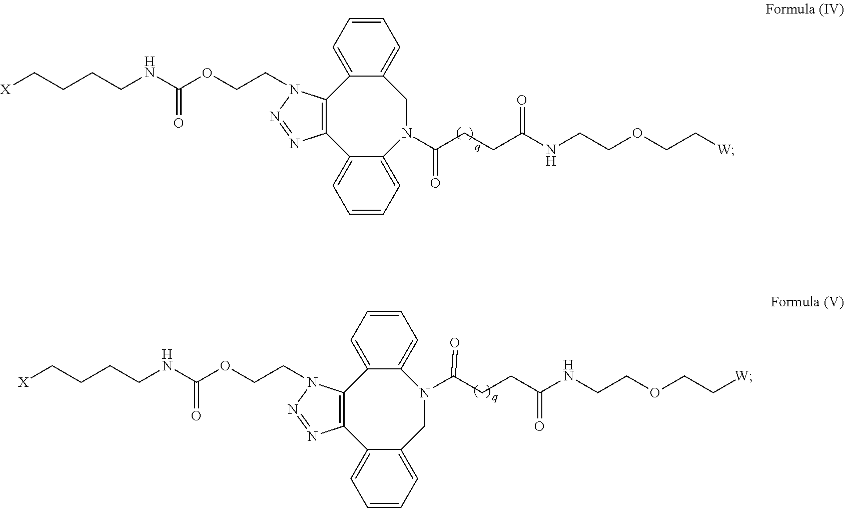

wherein:
W is a PEG group having an average molecular weight of about 25 kDa-35 kDa;
q is 1, 2, or 3;
X is an L-amino acid having the structure:

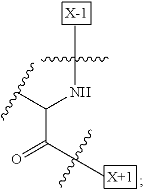

X−1 indicates the point of attachment to the preceding amino acid residue; and
X+1 indicates the point of attachment to the following amino acid residue.

Embodiment 20. The method of any one of embodiments 1-14, wherein the structure of Formula (I) has the structure of Formula (XII) or Formula (XIII), or is a mixture of Formula (XII) and Formula (XIII):

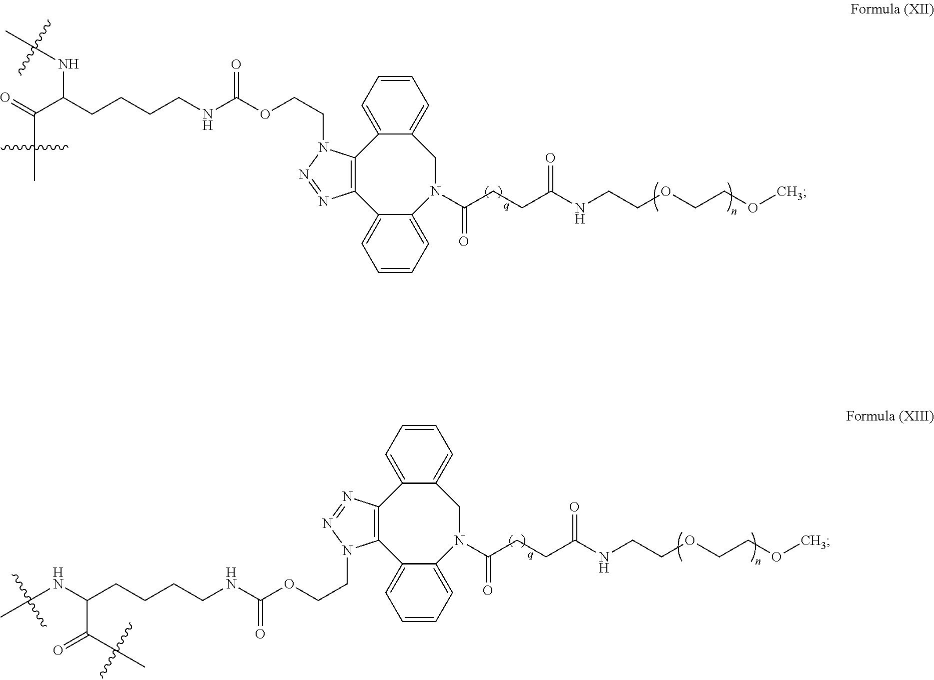

wherein:
n is an integer such that —(OCH₂CH₂)ₙ—OCH₃ has a molecular weight of about 30 kDa; q is 1, 2, or 3; and
the wavy lines indicate covalent bonds to amino acid residues within SEQ ID NO: 1 that are not replaced.

Embodiment 21. The method of any one of embodiments 1-20, wherein q is 1.

Embodiment 22. The method of any one of embodiments 1-20, wherein q is 2.

Embodiment 23. The method of any one of embodiments 1-20, wherein q is 3.

Embodiment 24. The method of any one of embodiments 1-23, wherein the average molecular weight is a number average molecular weight.

Embodiment 25. The method of any one of embodiments 1-23, wherein the average molecular weight is a weight average molecular weight.

Embodiment 26. The method of any one of embodiments 1-25, wherein the IL-2 conjugate is administered to the subject about once every two weeks, about once every three weeks, or about once every 4 weeks.

Embodiment 27. The method of any one of embodiments 1-26, wherein the IL-2 conjugate and cetuximab are administered to the subject about once every two weeks, about once every three weeks, or about once every 4 weeks.

Embodiment 28. The method of any one of embodiments 1-27, wherein the IL-2 conjugate is a pharmaceutically acceptable salt, solvate, or hydrate.

Embodiment 29. The method of any one of embodiments 1-28, wherein the initial dose of cetuximab is administered at a dose of about 400 mg/m², and subsequent doses of cetuximab are administered at a dose of about 250 mg/m².

Embodiment 30. The method of any one of embodiments 1-29, wherein cetuximab is administered before the IL-2 conjugate.

Embodiment 31. The method of any one of embodiments 1-30, wherein the IL-2 conjugate and cetuximab are administered separately.

Embodiment 32. The method of embodiment 31, wherein the IL-2 conjugate and cetuximab are administered sequentially.

Embodiment 33. The method of embodiment 32, wherein the IL-2 conjugate is administered after cetuximab.

Embodiment 34. The method of any one of embodiments 1-33, wherein the IL-2 conjugate is administered to the subject by subcutaneous administration.

Embodiment 35. The method of any one of embodiments 1-34, wherein the wherein the IL-2 conjugate and cetuximab are administered to the subject by subcutaneous administration.

Embodiment 36. The method of any one of embodiments 1-33, wherein the IL-2 conjugate is administered to the subject by intravenous administration.

Embodiment 37. The method of any one of embodiments 1-33 and 36, wherein the wherein the IL-2 conjugate and cetuximab are administered to the subject by intravenous administration.

Embodiment 38. The method of any one of embodiments 1-37, further comprising administering acetaminophen to the subject.

Embodiment 39. The method of any one of embodiments 1-38, further comprising administering diphenhydramine to the subject.

Embodiment 40. The method of any one of embodiments 1-39, further comprising administering ondansetron to the subject.

Embodiment 41. The method of any one of embodiments 38-40, wherein the acetaminophen, diphenhydramine, and/or ondansetron is administered to the subject before administering the IL-2 conjugate.

Embodiment 42. The method of any one of embodiments 38-40, wherein the acetaminophen, diphenhydramine, and/or ondansetron is administered to the subject before administering cetuximab.

Embodiment 43. An IL-2 conjugate for use in the method of any one of embodiments 1-42.

Embodiment 44. Use of an IL-2 conjugate for the manufacture of a medicament for the method of any one of embodiments 1-42.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C show the % cytotoxicity in CAL27 cells co-cultured with 3 separate donor human PBMCs and varying amounts of an IL-2 conjugate and cetuximab.

FIG. 2A shows the % cytotoxicity in CAL27 cells co-cultured with human PBMCs and varying amounts of an IL-2 conjugate and cetuximab.

FIG. 2B shows the % cytotoxicity in A431 cells co-cultured with human PBMCs and varying amounts of an IL-2 conjugate and cetuximab.

FIG. 3A shows the cytotoxic effect on A431 cells co-cultured with NK92 cells and treated varying amounts of an IL-2 conjugate and cetuximab.

FIG. 3B shows the % cytotoxicity on DLD-1 cells co-cultured with NK92 cells and treated varying amounts of an IL-2 conjugate and cetuximab.

FIG. 3C shows the % cytotoxicity on FaDu cells co-cultured with NK92 cells and treated varying amounts of an IL-2 conjugate and cetuximab.

FIG. 3D shows the % cytotoxicity on CAL27 cells co-cultured with NK92 cells and treated varying amounts of an IL-2 conjugate and cetuximab.

FIG. 4 shows the peripheral CD8+ T_{eff} cell counts in the indicated subjects treated with 16 μg/kg of the IL-2 conjugate in combination with cetuximab at specified times following administration of IL-2 conjugate.

FIG. 5 shows the peripheral NK cell counts in the indicated subjects treated with 16 μg/kg of the IL-2 conjugate in combination with cetuximab at specified times following administration of IL-2 conjugate.

FIG. 6 shows the peripheral CD4+T_{reg} cell counts in the indicated subjects treated with 16 μg/kg of the IL-2 conjugate in combination with cetuximab at specified times following administration of IL-2 conjugate.

FIG. 7 shows the eosinophil cell counts in the indicated subjects treated with 16 μg/kg of the IL-2 conjugate in combination with cetuximab at specified times following administration of IL-2 conjugate.

FIG. 8A shows the lymphocyte cell counts in the indicated subjects treated with 16 μg/kg of the IL-2 conjugate in combination with cetuximab at specified times following administration of IL-2 conjugate.

FIG. 8B shows the change in lymphocyte cell counts in the indicated subjects treated with 16 μg/kg of the IL-2 conjugate in combination with cetuximab at specified times following administration of IL-2 conjugate. Data is normalized to pre-treatment (C1D1) lymphocyte cell count.

FIG. 9 shows the peripheral CD8+ T_{eff} cell counts in the indicated subjects treated with 24 μg/kg of the IL-2 conjugate in combination with cetuximab at specified times following administration of IL-2 conjugate.

FIG. 10 shows the peripheral NK cell counts in the indicated subjects treated with 24 μg/kg of the IL-2 conjugate in combination with cetuximab at specified times following administration of IL-2 conjugate.

FIG. 11 shows the peripheral CD4+T_{reg} cell counts in the indicated subjects treated with 24 μg/kg of the IL-2 conjugate in combination with cetuximab at specified times following administration of IL-2 conjugate.

FIG. 12 shows the eosinophil cell counts in the indicated subjects treated with 24 μg/kg of the IL-2 conjugate in combination with cetuximab at specified times following administration of IL-2 conjugate.

FIG. 13A shows the lymphocyte cell counts in the indicated subjects treated with 24 μg/kg of the IL-2 conjugate in combination with cetuximab at specified times following administration of IL-2 conjugate.

FIG. 13B shows the change in lymphocyte cell counts in the indicated subjects treated with 24 μg/kg of the IL-2 conjugate in combination with cetuximab at specified times following administration of IL-2 conjugate. Data is normalized to pre-treatment (C1D1) lymphocyte cell count.

FIG. 14 shows serum levels of IFN-γ, IL-5, and IL-6 in the indicated subjects treated with 24 μg/kg of the IL-2 conjugate in combination with cetuximab at specified times following administration of IL-2 conjugate.

FIG. 15 shows the peripheral CD8+ T_{eff} cell counts in the indicated subjects treated with 32 μg/kg of the IL-2 conjugate in combination with cetuximab at specified times following administration of IL-2 conjugate.

FIG. 16 shows the peripheral NK cell counts in the indicated subjects treated with 32 μg/kg of the IL-2 conjugate in combination with cetuximab at specified times following administration of IL-2 conjugate.

FIG. 17 shows the peripheral CD4+T_{reg} cell counts in the indicated subjects treated with 32 μg/kg of the IL-2 conjugate in combination with cetuximab at specified times following administration of IL-2 conjugate.

FIG. 18 shows the eosinophil cell counts in the indicated subjects treated with 32 μg/kg of the IL-2 conjugate in combination with cetuximab at specified times following administration of IL-2 conjugate.

FIG. 19 shows serum levels of IFN-γ, IL-5, and IL-6 in the indicated subjects treated with 32 μg/kg of the IL-2 conjugate in combination with cetuximab at specified times following administration of the IL-2 conjugate. #: the maximum IFN-γ value of 1876 μg/mL for subject 4001-00116 at CsD1 POST was above the linear range of the assay.

FIG. 20A shows the peripheral NK cell count in subjects during Cycles 1 and 2. Subjects were treated with 16, 24, or 32 μg/kg of the IL-2 conjugate in combination with cetuximab.

FIG. 20B shows the peripheral NK cell count in subjects during Cycles 4 and 5. Subjects were treated with 16, 24, or 32 μg/kg of the IL-2 conjugate in combination with cetuximab.

FIG. 21A shows the peripheral CD8+ T_{eff} cell counts in subjects during Cycles 1 and 2. Subjects were treated with 16, 24, or 32 μg/kg of the IL-2 conjugate in combination with cetuximab.

FIG. 21B shows the peripheral CD8+ T_{eff} cell counts in subjects during Cycles 4 and 5. Subjects were treated with 16, 24, or 32 μg/kg of the IL-2 conjugate in combination with cetuximab.

FIG. 22A shows the peripheral CD4+T_{reg} cell counts in subjects during Cycles 1 and 2. Subjects were treated with 16, 24, or 32 μg/kg of the IL-2 conjugate in combination with cetuximab.

FIG. 22B shows the peripheral CD4+T_{reg} cell counts in subjects during Cycles 4 and 5. Subjects were treated with 16, 24, or 32 μg/kg of the IL-2 conjugate in combination with cetuximab.

FIG. 23A shows the eosinophil cell counts in subjects during Cycles 1 and 2. Subjects were treated with 16, 24, or 32 μg/kg of the IL-2 conjugate in combination with cetuximab.

FIG. 23B shows the eosinophil cell counts in subjects during Cycles 4 and 5. Subjects were treated with 16, 24, or 32 μg/kg of the IL-2 conjugate in combination with cetuximab.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the terms “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285(15): 11039-11044 (2010), the disclosure of which is incorporated herein by reference.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

As used herein, “dose-limiting toxicity” (DLT) is defined as an adverse event occurring within Day 1 through Day 29 (inclusive)±1 day of a treatment cycle that was not clearly or incontrovertibly solely related to an extraneous cause and that meets the criteria set forth in Example 2 for DLT.

As used herein, a “platinum-refractory” cancer is defined as a cancer in which the disease progresses during platinum-based therapy (i.e., patients do not achieve at least stable disease or a partial response to the platinum-based therapy), or the disease relapses within 6 months after the end of the platinum-based treatment.

As used herein, “treatment-naïve” refers to a subject who has never received treatment with a particular therapy. For example, a subject is treatment-naïve for cetuximab if the subject has never received treatment with cetuximab.

As used herein, “cetuximab” refers to the chimeric (mouse/human) anti-EGFR antibody marketed under the brand name “Erbitux” by Eli Lilly and Co.

Although various features of the invention may be described in the context of a single embodiment, the features may also be provided separately or in any suitable combination.

Conversely, although the invention may be described herein in the context of separate embodiments for clarity, the invention may also be implemented in a single embodiment.

### IL-2 Combination Therapy

Interleukin 2 (IL-2) is a pleiotropic type-1 cytokine whose structure comprises a 15.5 kDa four α-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2Rβ (also known as CD122), and IL-2Rγ (also known as CD132). Interaction of IL-2 with the IL-2Rα forms the “low-affinity” IL-2 receptor complex with a K_{d} of about 10⁻⁸ M. Interaction of IL-2 with IL-2Rβ and IL-2Rγ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (Treg) cells, mediate maintenance of immune homeostasis by suppression of effector cells such as CD4⁺ T cells, CD8⁺ T cells, B cells, NK cells, and NKT cells. In some instances, Treg cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4⁺ T cells produced a variety of autoimmune diseases in nude mice, whereas cotransfer of CD4⁺ CD25⁺ T cells suppressed the development of autoimmunity (Sakaguchi, et al., “Immunologic self-tolerance maintained by activated T cells expressing IL-2 receptor alpha-chains (CD25),” J. Immunol. 155(3): 1151-1164 (1995), the disclosure of which is incorporated herein by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8⁺ effector T (Teff) cells, NK cells, and NKT cells. CD8⁺ Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8⁺ Teff cells, target cancerous cells and pathogen-infected cells.

In some instances, IL-2 signaling is utilized to modulate T cell responses and subsequently for treatment of a cancer. For example, IL-2 is administered in a high-dose form to induce expansion of Teff cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome (VLS).

Adoptive cell therapy enables physicians to effectively harness a patient's own immune cells to fight diseases such as proliferative disease (e.g., cancer) as well as infectious disease. The effect of IL-2 signaling may be further enhanced by the presence of additional agents or methods in combination therapy.

One attractive therapy is combination of an IL-2 derivative with cetuximab. Cetuximab is a monoclonal antibody that binds to epidermal growth factor receptor (EGFR). EGFR is a cell surface receptor overexpressed in many types of cancer. Activation of EGFR promotes cell proliferation and survival, as well as angiogenesis, leading to tumor growth and metastasis. Cell growth and angiogenesis may be regulated by blocking the binding of EGFR to epidermal growth factor (EGF). By preventing EGF from binding to EGFR, the downstream signal transduction cascade is inhibited, leading to decreased cell growth. The same effect can be achieved by inhibiting transforming growth factor alpha (TGF-α) from binding to EGFR. The mechanism of action of cetuximab appears to include antibody dependent cell mediated cytotoxicity (Iannello, A. et al., Cancer Metastasis Rev. 2005, 24(4):487-99, the disclosure of which is incorporated herein by reference) in addition to EGFR blockade, which may contribute to its efficacy and may be further exploited. Cetuximab is indicated for the treatment of locally or regionally advanced squamous cell carcinoma of the head and neck in combination with radiation therapy; recurrent locoregional disease or metastatic squamous cell carcinoma of the head and neck in combination with platinum-based therapy with fluorouracil; and recurrent or metastatic squamous cell carcinoma of the head and neck.

Head and neck squamous cell carcinoma (HNSCC) is the ninth leading cancer by incidence worldwide and constitutes 90% of all head and neck cancers (Gupta et al. Oncology, 2016, 91(1):13-23, the disclosure of which is incorporated herein by reference). HNSCC is a biologically diverse and genomically heterogeneous disease that arises from the squamous mucosal lining of the upper aerodigestive tract, including the lip and oral cavity, nasal cavity, paranasal sinuses, nasopharynx, oropharynx, larynx and hypopharynx. A large number of patients with head and neck cancer initially present with locally advanced, Stage III/IV disease that is initially treated with combinations of chemotherapy, radiation and/or surgery. This initial treatment can result in disease control rates ranging between 33% and 86% of patients. Patients who progress after initial therapy require subsequent treatment for recurrent (R) or metastatic (M) disease.

### IL-2 Conjugates

Provided herein are methods of treating HNSCC in a subject in need thereof, comprising administering to the subject an IL-2 conjugate in combination with one or more additional agents.

In some embodiments, the IL-2 sequence comprises the sequence of SEQ ID NO: 1:

(SEQ ID NO: 1)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT

wherein the amino acid at position P64 is replaced by the structure of Formula (I):

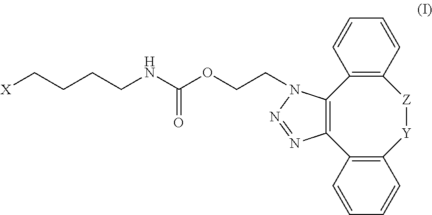

wherein:
Z is CH₂ and Y is

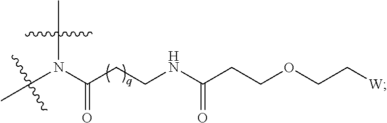

Y is CH₂ and Z is

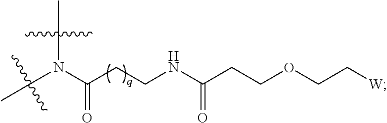

Z is CH₂ and Y is

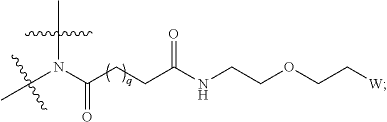

for
Y is CH₂ and Z is

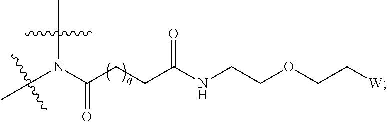

W is a PEG group having an average molecular weight of about 25 kDa-35 kDa;
q is 1, 2, or 3;
X is an L-amino acid having the structure:

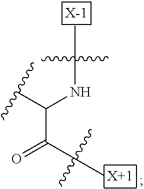

X−1 indicates the point of attachment to the preceding amino acid residue; and
X+1 indicates the point of attachment to the following amino acid residue.

In any of the embodiments or variations of Formula (I) described herein, the IL-2 conjugate is a pharmaceutically acceptable salt, solvate, or hydrate. In some embodiments, the IL-2 conjugate is a pharmaceutically acceptable salt. In some embodiments, the IL-2 conjugate is a solvate. In some embodiments, the IL-2 conjugate is a hydrate.

In any of the embodiments or variations of Formula (I) described herein and pharmaceutical compositions comprising the same, average molecular weight encompasses both weight average molecular weight and number average molecular weight; in other words, for example, both a 30 kDa number average molecular weight and a 30 kDa weight average molecular weight qualify as a 30 kDa molecular weight. In some embodiments, the average molecular weight is weight average molecular weight. In other embodiments, the average molecular weight is number average molecular weight. It is understood that in the methods provided herein, administering an IL-2 conjugate as described herein to a subject comprises administering more than a single molecule of IL-2 conjugate; as such, use of the term “average” to describe the molecular weight of the PEG group refers to the average molecular weight of the PEG groups of the IL-2 conjugate molecules in a dose administered to the subject.

In some embodiments of Formula (I), Z is CH₂ and Y is

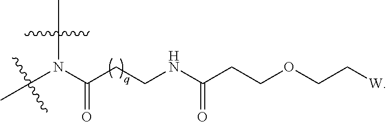

In some embodiments of Formula (I), Y is CH₂ and Z is

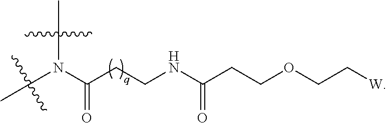

In some embodiments of Formula (I), Z is CH₂ and Y is

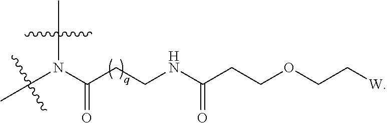

In some embodiments of Formula (I), Y is CH₂ and Z is

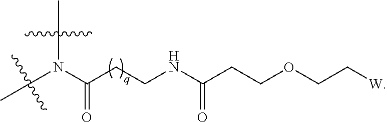

In some embodiments of Formula (I), q is 1. In some embodiments of Formula (I), q is 2. In some embodiments of Formula (I), q is 3.

In some embodiments of Formula (I), W is a PEG group having an average molecular weight of about 25 kDa. In some embodiments of Formula (I), W is a PEG group having an average molecular weight of about 30 kDa. In some embodiments of Formula (I), W is a PEG group having an average molecular weight of about 35 kDa.

In some embodiments of Formula (I), q is 1 and structure of Formula (I) is the structure of Formula (Ia):

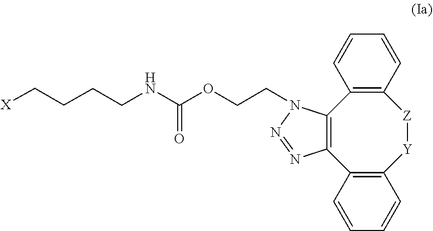

wherein:
Z is CH₂ and Y is

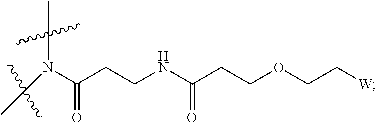

Y is CH₂ and Z is

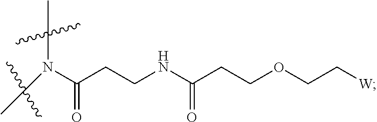

Z is CH₂ and Y is

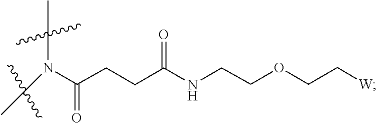

or
Y is CH₂ and Z is

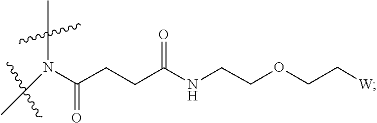

W is a PEG group having an average molecular weight of about 25 kDa-35 kDa;
X is an L-amino acid having the structure:

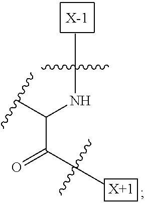

X−1 indicates the point of attachment to the preceding amino acid residue; and
X+1 indicates the point of attachment to the following amino acid residue.

In some embodiments of Formula (Ia), Z is CH₂ and Y is

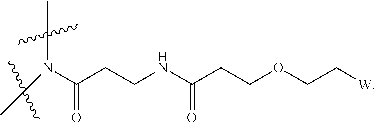

In some embodiments of Formula (Ia), Y is CH₂ and Z is

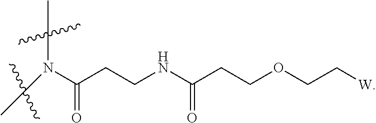

In other embodiments of Formula (Ia), Z is CH₂ and Y is

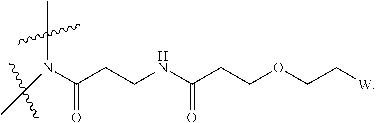

In some embodiments of Formula (Ia), Y is CH₂ and Z is

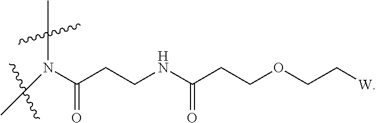

In some embodiments of Formula (Ia), the PEG group has an average molecular weight of about 30 kDa.

In some embodiments, the IL-2 conjugate comprises the sequence of SEQ ID NO: 2: PTSSSTKKTQLQLEHLLLDLQMILNGINNYKNPKLTRMLTFKFYMPKKATELKHLQCLE EELK[AzK_L1_PEG30kD]LEEVLNLAQSKNFHLRPRDLISNINVIVLELKGSETTFMCEY ADETATIVEFLNRWITFSQSIISTLT (SEQ ID NO: 2)
wherein [AzK_L1_PEG30kD] is N6-((2-azidoethoxy)-carbonyl)-L-lysine stably-conjugated to PEG via DBCO-mediated click chemistry to form a compound comprising a structure of Formula (IV) or Formula (V), wherein q is 1 (such as Formula (IVa) or Formula (Va)), and wherein the PEG group has an average molecular weight of about 25-35 kiloDaltons (e.g., about 30 kDa), capped with a methoxy group. The term “DBCO” means a chemical moiety comprising a dibenzocyclooctyne group, such as comprising the mPEG-DBCO compound illustrated in Schemes 1 and 2 of Example 1.

The ratio of regioisomers generated from the click reaction is about 1:1 or greater than 1:1.

PEGs will typically comprise a number of (OCH₂CH₂) monomers (or (CH₂CH₂O) monomers, depending on how the PEG is defined). In some embodiments, the number of (OCH₂CH₂) monomers (or (CH₂CH₂O) monomers) is such that the average molecular weight of the PEG group is about 30 kDa.

In some instances, the PEG is an end-capped polymer, that is, a polymer having at least one terminus capped with a relatively inert group, such as a lower C₁₋₆ alkoxy group, or a hydroxyl group. In some embodiments, the PEG group is a methoxy-PEG (commonly referred to as mPEG), which is a linear form of PEG wherein one terminus of the polymer is a methoxy (—OCH₃) group, and the other terminus is a hydroxyl or other functional group that can be optionally chemically modified.

In some embodiments, the PEG group is a linear or branched PEG group. In some embodiments, the PEG group is a linear PEG group. In some embodiments, the PEG group is a branched PEG group. In some embodiments, the PEG group is a methoxy PEG group. In some embodiments, the PEG group is a linear or branched methoxy PEG group. In some embodiments, the PEG group is a linear methoxy PEG group. In some embodiments, the PEG group is a branched methoxy PEG group. For example, included within the scope of the present disclosure are IL-2 conjugates comprising a PEG group having a molecular weight of 30,000 Da±3,000 Da, or 30,000 Da±4,500 Da, or 30,000 Da±5,000 Da.

In some embodiments, the IL-2 conjugate comprises the amino acid sequence of SEQ ID NO: 1 in which the amino acid residue P64 is replaced by the structure of Formula (IV) or Formula (V), or a mixture of Formula (IV) and Formula (V):

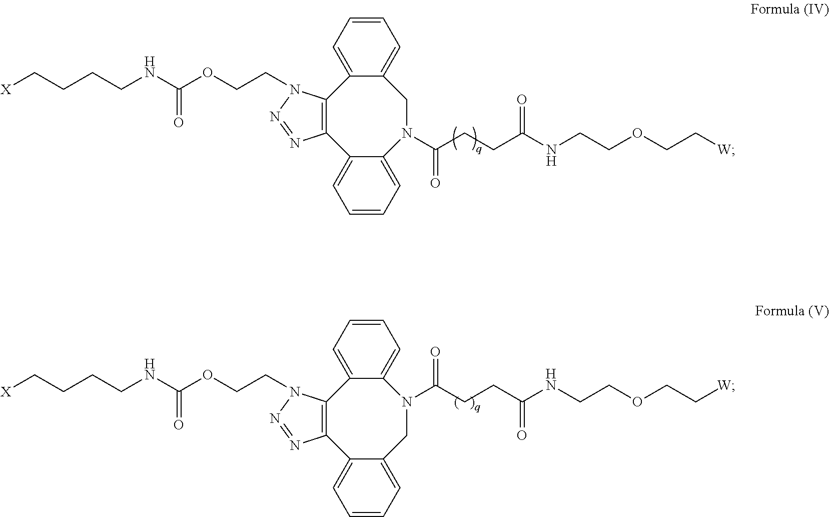

wherein:
W is a PEG group having an average molecular weight of about 25 kDa-35 kDa;
q is 1, 2, or 3; and
X has the structure:

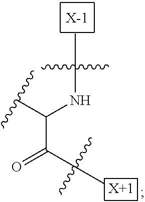

X−1 indicates the point of attachment to the preceding amino acid residue; and
X+1 indicates the point of attachment to the following amino acid residue.

In some embodiments of Formula (IV) or Formula (V), or a mixture of Formula (IV) or Formula (V), q is 1. In some embodiments of Formula (IV) or Formula (V), or a mixture of Formula (IV) or Formula (V), q is 2. In some embodiments of Formula (IV) or Formula (V), or a mixture of Formula (IV) or Formula (V), q is 3.

In some embodiments of Formula (IV) or Formula (V), or a mixture of Formula (IV) or Formula (V), W is a PEG group having an average molecular weight of about 25 kDa. In some embodiments of Formula (IV) or Formula (V), or a mixture of Formula (IV) or Formula (V), W is a PEG group having an average molecular weight of about 30 kDa. In some embodiments of Formula (IV) or Formula (V), or a mixture of Formula (IV) or Formula (V), W is a PEG group having an average molecular weight of about 35 kDa.

In any of the embodiments described herein, the structure of Formula (I) has the structure of Formula (IV) or Formula (V), or is a mixture of Formula (IV) and Formula (V). In some embodiments, the structure of Formula (I) has the structure of Formula (IV). In some embodiments, the structure of Formula (I) has the structure of Formula (V). In some embodiments, the structure of Formula (I) is a mixture of Formula (IV) and Formula (V).

In some embodiments of Formula (IV) or Formula (V), or a mixture of Formula (IV) and Formula (V), q is 1, the structure of Formula (IV) is the structure of Formula (IVa), and the structure of Formula (V) is the structure of Formula (Va):

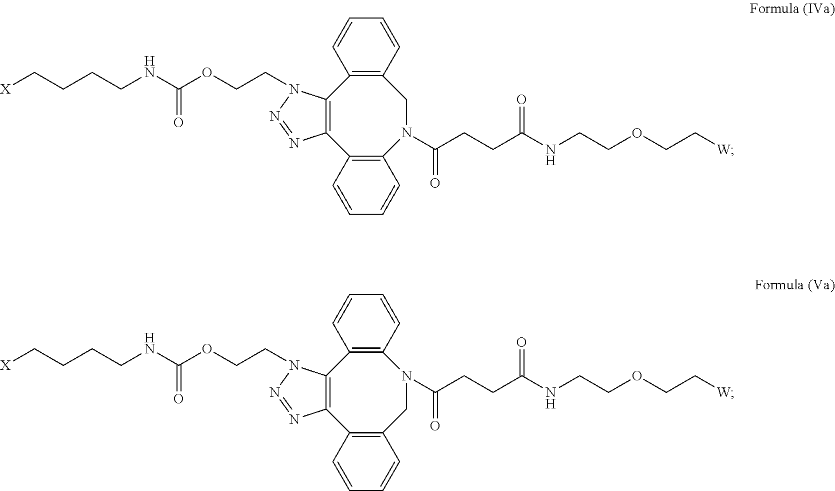

wherein:
W is a PEG group having an average molecular weight of about 25 kDa-35 kDa; and
X has the structure:

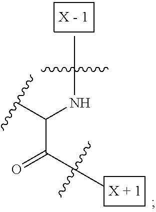

X−1 indicates the point of attachment to the preceding amino acid residue; and
X+1 indicates the point of attachment to the following amino acid residue.

In some embodiments of Formula (IVa) or Formula (Va), or a mixture of Formula (IVa) and Formula (Va), the PEG group has an average molecular weight of about 30 kDa.

In any of the embodiments described herein, the structure of Formula (I) has the structure of Formula (IVa) or Formula (Va), or is a mixture of Formula (IVa) and Formula (Va).

In some embodiments, the structure of Formula (I) has the structure of Formula (IVa). In some embodiments, the structure of Formula (I) has the structure of Formula (Va). In some embodiments, the structure of Formula (I) is a mixture of Formula (IVa) and Formula (Va).

In some embodiments, the IL-2 conjugate comprises the amino acid sequence of SEQ ID NO: 1 in which the amino acid residue P64 is replaced by the structure of Formula (XII) or Formula (XIII), or a mixture of Formula (XII) and Formula (XIII):

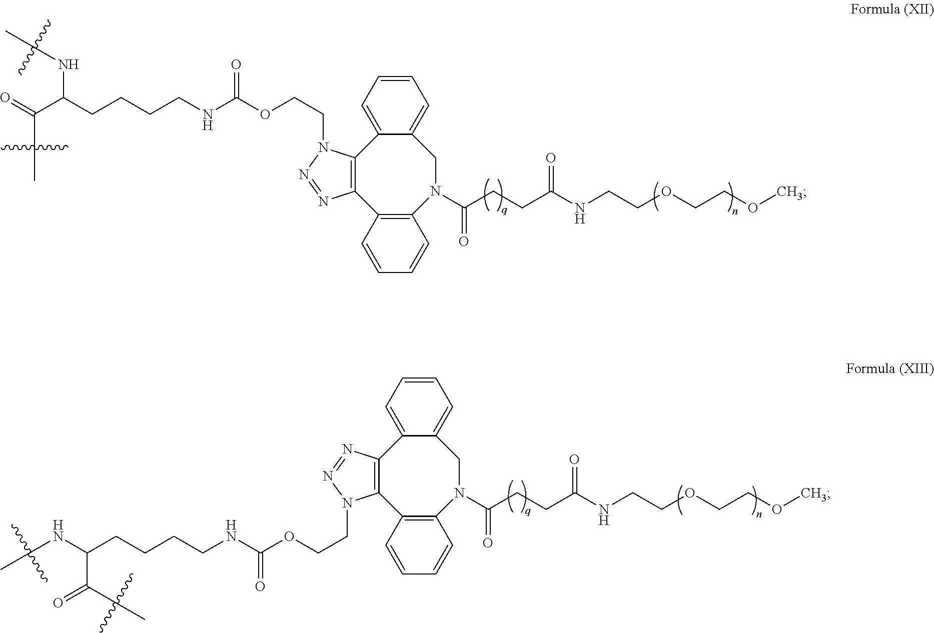

wherein:
n is is an integer such that —(OCH₂CH₂)ₙ—OCH₃ has a molecular weight of about 25 kDa-35 kDa;
q is 1, 2, or 3; and
the wavy lines indicate convalent bonds to amino acid residues within SEQ ID NO: 1 that are not replaced.

In some embodiments of Formula (XII) or Formula (XIII), or a mixture of Formula (XII) and Formula (XIII), q is 1. In some embodiments of Formula (XII) or Formula (XIII), or a mixture of Formula (XII) and Formula (XIII), q is 2. In some embodiments of Formula (XII) or Formula (XIII), or a mixture of Formula (XII) and Formula (XIII), q is 3.

In some embodiments of Formula (XII) or Formula (XIII), or a mixture of Formula (XII) and Formula (XIII), n is is an integer such that —(OCH₂CH₂)ₙ—OCH₃ has a molecular weight of about 30 kDa.

In any of the embodiments described herein, the structure of Formula (I) has the structure of Formula (XII) or Formula (XIII), or is a mixture of Formula (XII) and Formula (XIII). In some embodiments, the structure of Formula (I) has the structure of Formula (XII). In some embodiments, the structure of Formula (I) has the structure of Formula (XIII). In some embodiments, the structure of Formula (I) is a mixture of Formula (XII) and Formula (XIII).

In some embodiments of Formula (XII) or Formula (XIII), or a mixture of Formula (XII) and Formula (XIII), q is 1, the structure of Formula (XII) is the structure of Formula (XIIa), and the structure of Formula (XIII) is the structure of Formula (XIIIa):

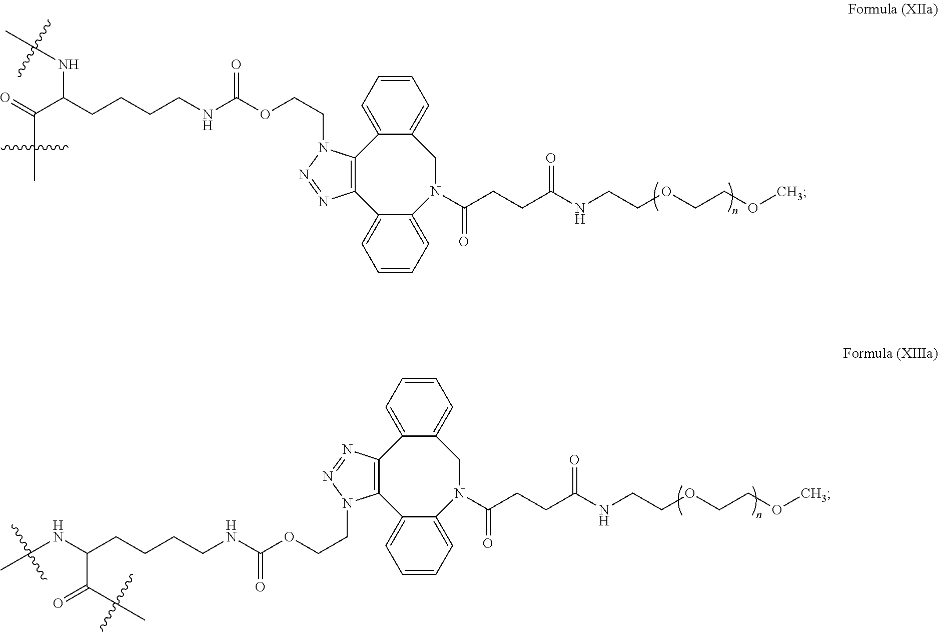

wherein:
n is is an integer such that —(OCH₂CH₂)ₙ—OCH₃ has a molecular weight of about 25 kDa-35 kDa; and
the wavy lines indicate convalent bonds to amino acid residues within SEQ ID NO: 1 that are not replaced.

In some embodiments of Formula (XIIa) or Formula (XIIIa), or a mixture of Formula (XIIa) and Formula (XIIIa), n is is an integer such that —(OCH₂CH₂)ₙ—OCH₃ has a molecular weight of about 30 kDa.

In any of the embodiments described herein, the structure of Formula (I) has the structure of Formula (XIIa) or Formula (XIIIa), or is a mixture of Formula (XIIa) and Formula (XIIIa). In some embodiments, the structure of Formula (I) has the structure of Formula (XIIa).

In some embodiments, the structure of Formula (I) has the structure of Formula (XIIIa). In some embodiments, the structure of Formula (I) is a mixture of Formula (XIIa) and Formula (XIIIa).

In some embodiments, the IL-2 conjugate comprises the amino acid sequence of SEQ ID NO: 1 in which the amino acid residue P64 is replaced by the structure of Formula (XIV) or Formula (XV), or a mixture of Formula (XIV) and Formula (XV):

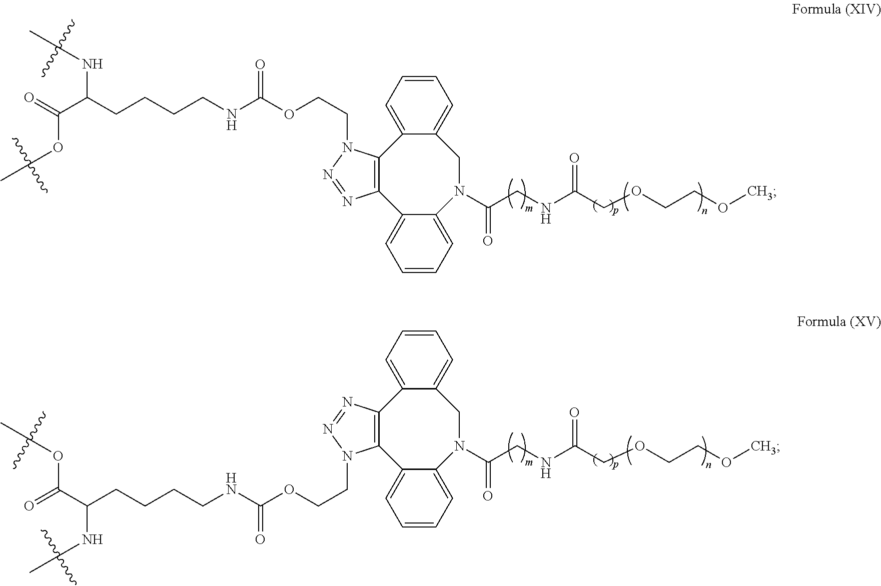

wherein:
m is an integer from 0 to 20;
p is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XIV) or Formula (XV), or a mixture of Formula (XIV) and Formula (XV), n is an integer such that the PEG group has an average molecular weight of about 30 kDa.

In some embodiments, m is an integer from 0 to 15. In some embodiments, m is an integer from 0 to 10. In some embodiments, m is an integer from 0 to 5. In some embodiments, m is an integer from 1 to 5. In some embodiments, m is 1. In some embodiments, m is 2. In some embodiments, m is 3. In some embodiments, m is 4. In some embodiments, m is 5.

In some embodiments, p is an integer from 0 to 15. In some embodiments, p is an integer from 0 to 10. In some embodiments, p is an integer from 0 to 5. In some embodiments, p is an integer from 1 to 5. In some embodiments, p is 1. In some embodiments, p is 2. In some embodiments, p is 3. In some embodiments, p is 4. In some embodiments, p is 5.

In some embodiments, m and p are each 2.

In any of the embodiments described herein, the structure of Formula (I) has the structure of Formula (XIV) or Formula (XV), or is a mixture of Formula (XIV) and Formula (XV). In some embodiments, the structure of Formula (I) has the structure of Formula (XIV). In some embodiments, the structure of Formula (I) has the structure of Formula (XV). In some embodiments, the structure of Formula (I) is a mixture of Formula (XIV) and Formula (XV).

In some embodiments, the IL-2 conjugate comprises the amino acid sequence of SEQ ID NO: 1 in which the amino acid residue P64 is replaced by the structure of Formula (XVI) or Formula (XVII), or a mixture of Formula (XVI) and Formula (XVII):

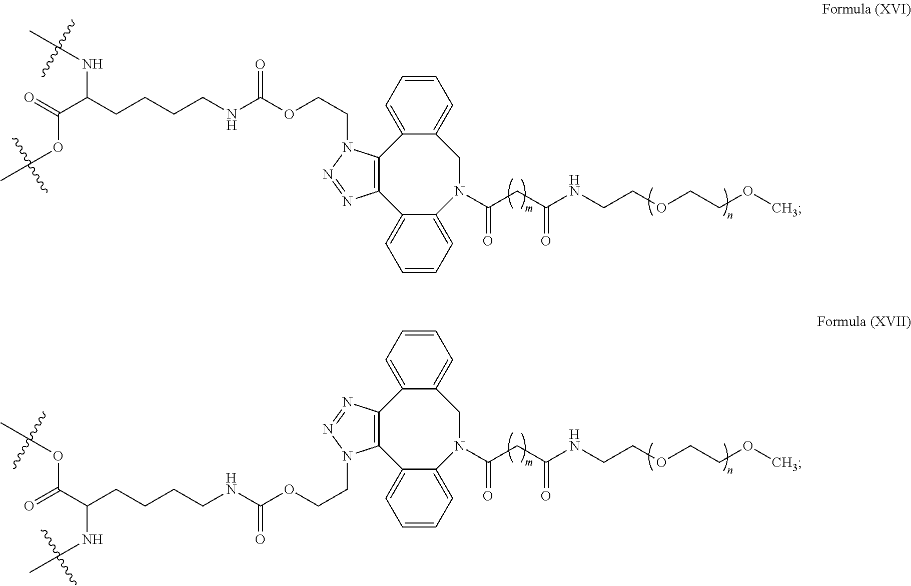

wherein:
H is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XVI) or Formula (XVII), or a mixture of Formula (XVI) and Formula (XVII), n is an integer such that the PEG group has an average molecular weight of about 30 kDa.

In some embodiments, m is an integer from 0 to 15. In some embodiments, m is an integer from 0 to 10. In some embodiments, is an integer from 0 to 5. In some embodiments, n is an integer from 1 to 5. In some embodiments, m is 1. In some embodiments, 2 is 2. In some embodiments, m is 3. In some embodiments, m is 4. In some embodiments, m is 5.

In any of the embodiments described herein, the structure of Formula (I) has the structure of Formula (XVI) or Formula (XVII), or is a mixture of Formula (XVI) and Formula (XVII). In some embodiments, the structure of Formula (I) has the structure of Formula (XVI). In some embodiments, the structure of Formula (I) has the structure of Formula (XVII). In some embodiments, the structure of Formula (I) is a mixture of Formula (XVI) and Formula (XVII).

### Conjugation Chemistry

In some embodiments, the IL-2 conjugates described herein can be prepared by a conjugation reaction comprising a 1,3-dipolar cycloaddition reaction. In some embodiments, the 1,3-dipolar cycloaddition reaction comprises reaction of an azide and an alkyne (“Click” reaction). In some embodiments, a conjugation reaction described herein comprises the reaction outlined in Scheme I, wherein X is an unnatural amino acid at position P64 of SEQ ID NO: 1.

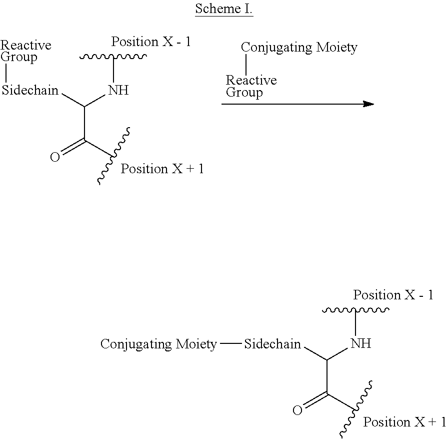

In some embodiments, the conjugating moiety comprises a PEG group as described herein. In some embodiments, a reactive group comprises an alkyne or azide.

In some embodiments, a conjugation reaction described herein comprises the reaction outlined in Scheme II, wherein X is an unnatural amino acid at position P64 of SEQ ID NO: 1.

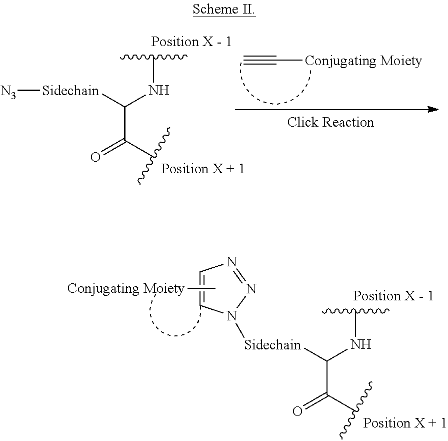

In some embodiments, a conjugation reaction described herein comprises the reaction outlined in Scheme III, wherein X is an unnatural amino acid at position P64 of SEQ ID NO: 1.

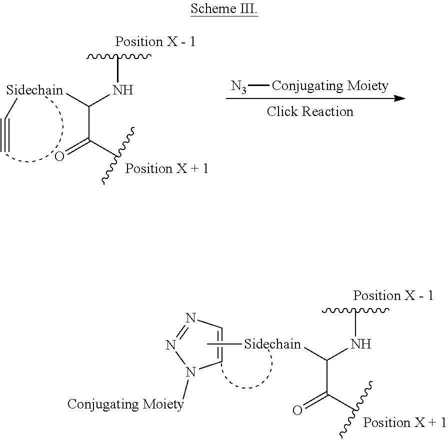

In some embodiments a conjugation reaction described herein comprises the reaction outlined in Scheme IV, wherein X is an unnatural amino acid at position P64 of SEQ ID NO: 1.

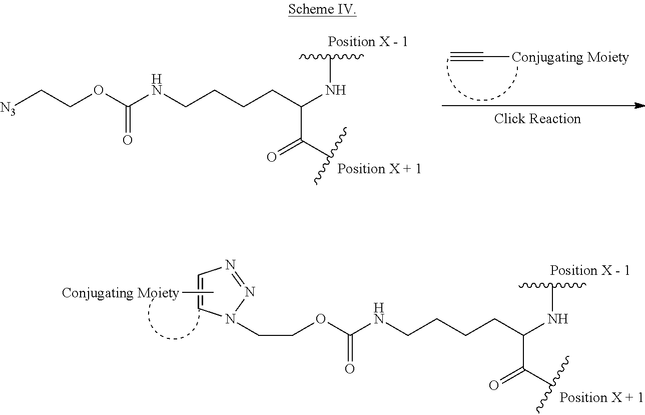

In some embodiments, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods known to those of ordinary skill in the art. Exemplary reactions are shown in Schemes V and VI.

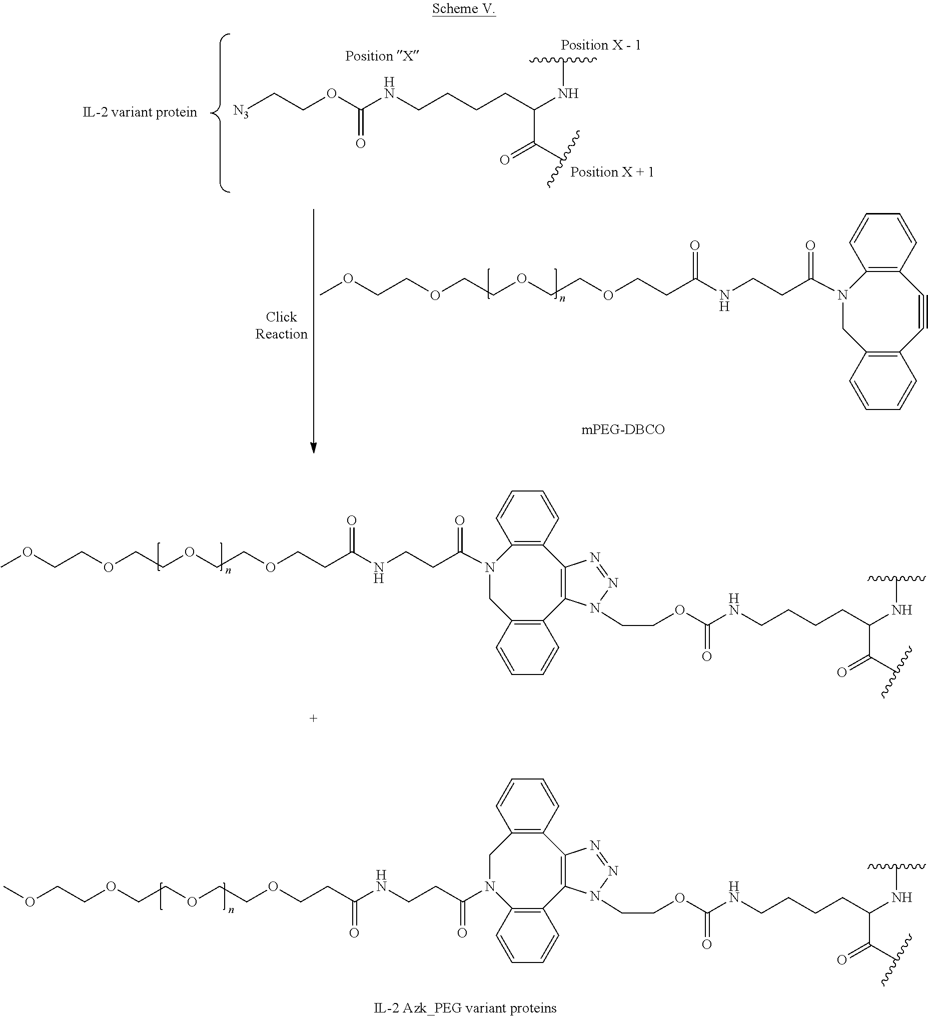

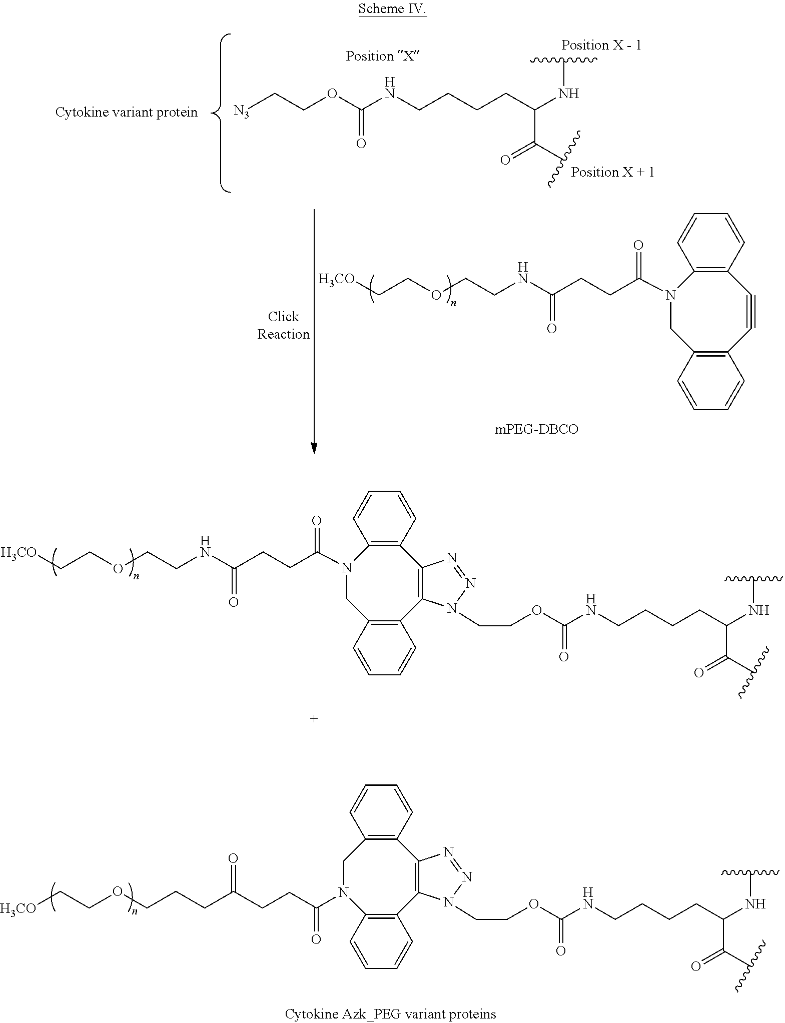

Conjugation reactions such as a click reaction described herein may generate a single regioisomer, or a mixture of regioisomers. In some instances the ratio of regioisomers is about 1:1. In some instances the ratio of regioisomers is about 2:1. In some instances the ratio of regioisomers is about 1.5:1. In some instances the ratio of regioisomers is about 1.2:1. In some instances the ratio of regioisomers is about 1.1:1. In some instances the ratio of regioisomers is greater than 1:1.

### IL-2 Polypeptide Production

In some instances, the IL-2 conjugates described herein, either containing a natural amino acid mutation or an unnatural amino acid mutation, are generated recombinantly or are synthesized chemically. In some instances, IL-2 conjugates described herein are generated recombinantly, for example, either by a host cell system, or in a cell-free system.

In some instances, IL-2 conjugates are generated recombinantly through a host cell system. In some cases, the host cell is a eukaryotic cell (e.g., mammalian cell, insect cells, yeast cells or plant cell) or a prokaryotic cell (e.g., Gram-positive bacterium or a Gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cells include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS 115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Machi™, DH10B™, TOP10, DH5a, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable polynucleic acid molecules or vectors for the production of an IL-2 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway®pYES-DEST52 vector, pBAD-DEST49 Gateway® destination vector, pAO815 Pichia vector, pFLD1 Pichia pastoris (K phaffii) vector, pGAPZA, B, & C Pichia pastoris (K. phaffii) vector, pPIC3.5K Pichia vector, pPIC6A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is used for the production of an IL-2 polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, tRNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631, the disclosure of each of which is incorporated herein by reference. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, an IL-2 polypeptide is generated as a circularly permuted form, either via an expression host system or through a cell-free system.

### Production of Cytokine Polypeptide Comprising an Unnatural Amino Acid

An orthogonal or expanded genetic code can be used in the present disclosure, in which one or more specific codons present in the nucleic acid sequence of an IL-2 polypeptide are allocated to encode the unnatural amino acid so that it can be genetically incorporated into the IL-2 by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464(7287): 441-444 (2010), the disclosure of which is incorporated herein by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113(38): E5588-5597 (2016), the disclosure of which is incorporated herein by reference. In some cases, the recoded codon is as described in Ostrov et al., “Design, synthesis, and testing toward a 57-codon genome,” Science 353(6301): 819-822 (2016), the disclosure of which is incorporated herein by reference.

In some instances, unnatural nucleic acids are utilized leading to incorporation of one or more unnatural amino acids into the IL-2. Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993; the disclosure of each of which is incorporated herein by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d]pyrimidin-5-yl, 2-amino-4-oxopyrolo [2, 3-d] pyrimidin-5-yl, 2-amino-4-oxopyrrolo [2.3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′.

Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050; the disclosure of each of which is incorporated herein by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337); the disclosure of each of which is incorporated herein by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or 0) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414). The disclosure of each reference listed in this paragraph is incorporated herein by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181, the disclosure of each of which is incorporated herein by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]m CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, the disclosure of each of which is incorporated herein by reference.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂-O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393. The disclosure of each reference listed in this paragraph is incorporated herein by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179; the disclosure of each of which is incorporated herein by reference.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179, the disclosure of which is incorporated herein by reference). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference.

See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941. The disclosure of each reference listed in this paragraph is incorporated herein by reference.

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 5SICS, d5SICS, NaM, dNaM, CNMO, dCNMO, and combinations thereof. In some embodiments, unnatural nucleotides include:

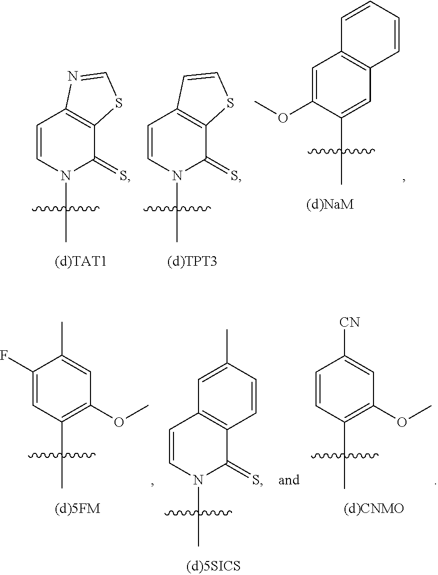

Exemplary unnatural base pairs include: (d)TPT3-(d)NaM; (d)₅SICS-(d)NaM; (d)CNMO-(d)TAT1; (d)NaM-(d)TAT1; (d)CNMO-(d)TPT3; and (d)₅FM-(d)TAT1.

Other examples of unnatural nucleotides capable of forming unnatural UBPs that may be used to prepare the IL-2 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010; the disclosure of each of which is incorporated herein by reference. In some embodiments, unnatural nucleotides include:

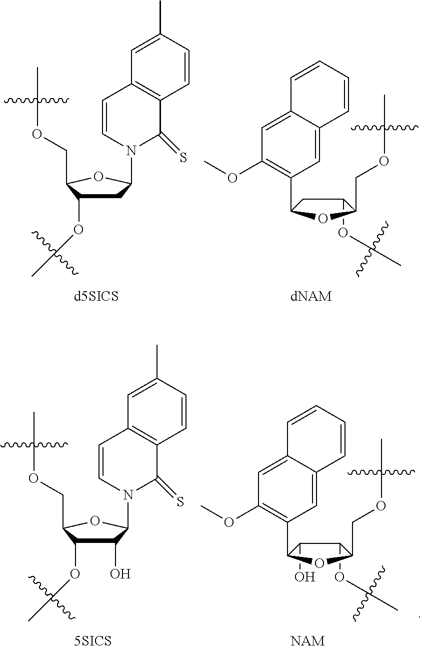

In some embodiments, the unnatural nucleotides that may be used to prepare the IL-2 conjugates disclosed herein may be derived from a compound of the formula

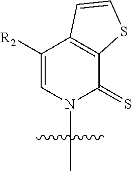

wherein R₂ is selected from the group consisting of hydrogen, alkyl, alkenyl, alkynyl, methoxy, methanethiol, methaneseleno, halogen, cyano, and azido; and
the wavy line indicates a bond to a ribosyl or 2′-deoxyribosyl, wherein the 5′-hydroxy group of the ribosyl or 2′-deoxyribosyl moiety is in free form, is connected to a monophosphate, diphosphate, triphosphate, α-thiotriphosphate, β-thiotriphosphate, or γ-thiotriphosphate group, or is included in an RNA or a DNA or in an RNA analog or a DNA analog.

In some embodiments, the unnatural nucleotides that may be used to prepare the IL-2 conjugates disclosed herein may be derived from a compound of the Formula

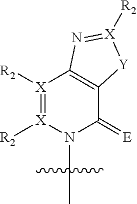

wherein:
each X is independently carbon or nitrogen;
R₂ is absent when X is nitrogen, and is present when X is carbon and is independently hydrogen, alkyl, alkenyl, alkynyl, methoxy, methanethiol, methaneseleno, halogen, cyano, or azide;
Y is sulfur, oxygen, selenium, or secondary amine;
E is oxygen, sulfur, or selenium; and
the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, is connected to a mono-phosphate, diphosphate, triphosphate, α-thiotriphosphate, β-thiotriphosphate, or γ-thiotriphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R₂ is present when X is carbon. In some embodiments, R² is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂═CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-2 conjugates disclosed herein may be derived from

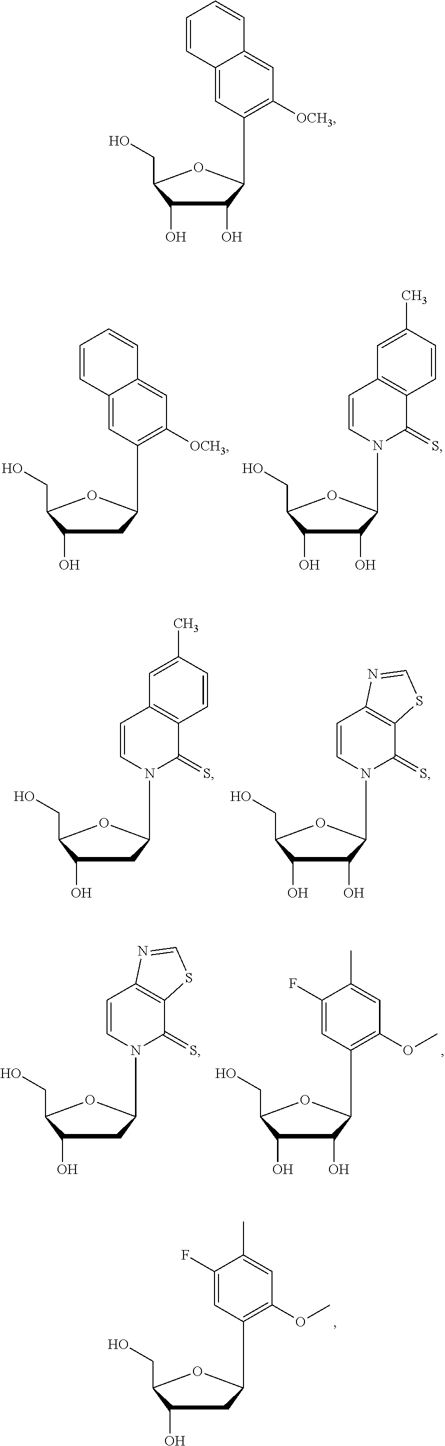

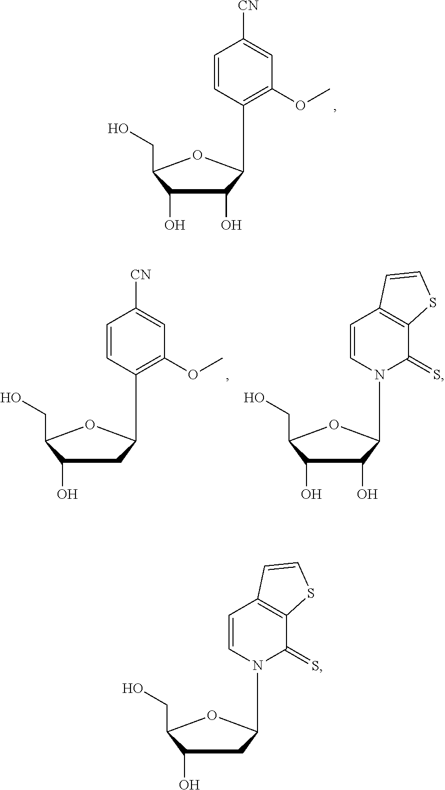

In some embodiments, the unnatural nucleotides that may be used to prepare the IL-2 conjugates disclosed herein include

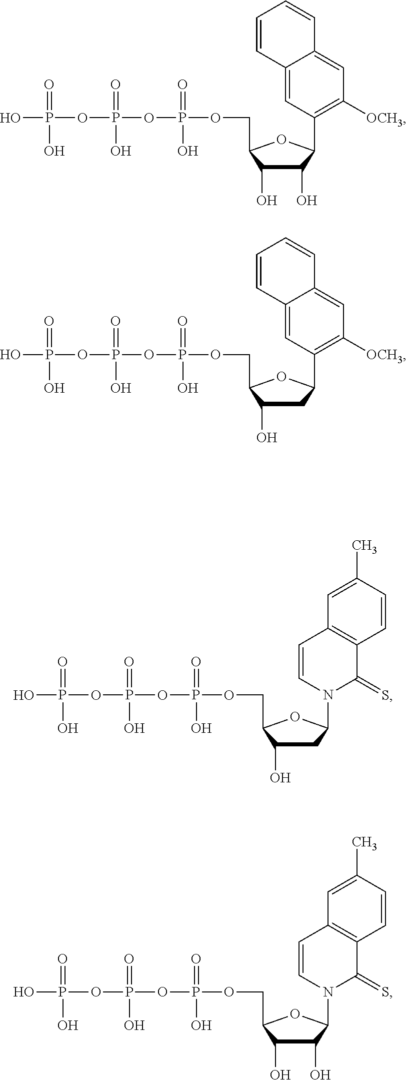

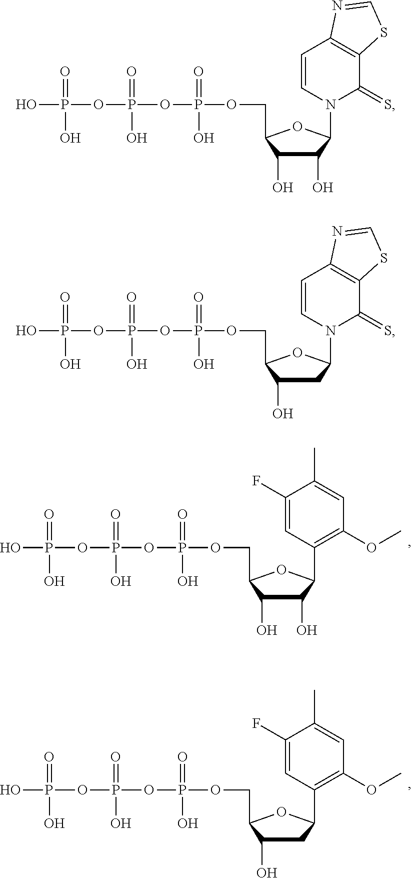

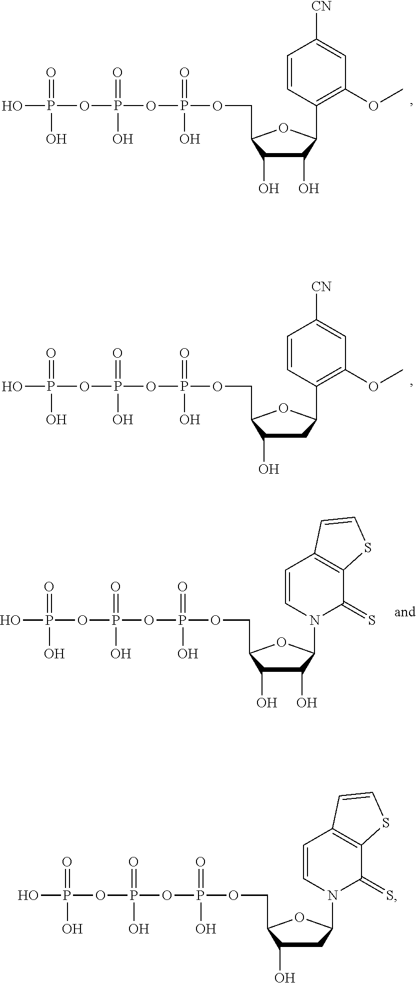

or salts thereof.

In some embodiments, an unnatural base pair generate an unnatural amino acid described in Dumas et al., “Designing logical codon reassignment—Expanding the chemistry in biology,” Chemical Science, 6: 50-69 (2015), the disclosure of which is incorporated herein by reference.

In some embodiments, the unnatural amino acid is incorporated into the cytokine (e.g., the IL polypeptide) by a synthetic codon comprising an unnatural nucleic acid. In some instances, the unnatural amino acid is incorporated into the cytokine by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the cytokine (e.g., the IL polypeptide) by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. In some instances, the unnatural amino acid is incorporated into the cytokine (e.g., the IL polypeptide) by a Mj-TyrRS/tRNA pair. Exemplary UAAs that can be incorporated by a Mj-TyrRS/tRNA pair include, but are not limited to, para-substituted phenylalanine derivatives such as p-aminophenylalanine andp-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; and o-nitrobenzyltyrosine.

In some instances, the unnatural amino acid is incorporated into the cytokine (e.g., the IL polypeptide) by a Ec-Tyr/tRNA_{CUA} or a Ec-Leu/tRNA_{CUA} pair. Exemplary UAAs that can be incorporated by a Ec-Tyr/tRNA_{CUA} or a Ec-Leu/tRNA_{CUA} pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into the cytokine (e.g., the IL polypeptide) by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans.

Exemplary UAAs that can be incorporated by a pyrrolysyl-tRNA pair include, but are not limited to, amide and carbamate substituted lysines such as 2-amino-6-((R)-tetrahydrofuran-2-carboxamido)hexanoic acid, N-ε-D-prolyl-L-lysine, and N-ε-cyclopentyloxycarbonyl-L-lysine; N-ε-Acryloyl-L-lysine; N-ε-[(1-(6-nitrobenzo[d][1,3]dioxol-5-yl)ethoxy)carbonyl]-L-lysine; and N-ε-(1-methylcyclopro-2-enecarboxamido)lysine. In some embodiments, the IL-2 conjugates disclosed herein may be prepared by use of M. mazei tRNA which is selectively charged with a non-natural amino acid such as N6-((2-azidoethoxy)-carbonyl)-L-lysine (AzK) by the M. barkeri pyrrolysyl-tRNA synthetase (Mb PylRS). Other methods are known to those of ordinary skill in the art, such as those disclosed in Zhang et al., Nature 2017, 551(7682): 644-647, the disclosure of which is incorporated herein by reference.

In some instances, an unnatural amino acid is incorporated into a cytokine described herein (e.g., the IL polypeptide) by a synthetase disclosed in U.S. Pat. Nos. 9,988,619 and 9,938,516, the disclosure of each of which is incorporated herein by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-2 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTTT (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551(7682): 644-647; Malyshev et al. Nature 2014 (509(7500), 385-388; and Zhang et al. Proc Natl Acad Sci USA, 2017, 114:1317-1322, the disclosure of each of which is incorporated herein by reference.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322; the disclosure of each of which is incorporated herein by reference.

In some embodiments are provided methods of preparing the IL-2 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E. coli TyrRS (Ec-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E. coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E. coli TyrRS (Ec-Tyr)/B. stearothermophilus, or M. mazei.

In some embodiments, the IL-2 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-2 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), will be incorporated, (c) a plasmid encoding a tRNA derived from M. mazei and which comprises an unnatural nucleotide to provide a recognized anticodon (to the codon of the IL-2 variant) in place of its native sequence, and (d) a plasmid encoding a M. barkeri derived pyrrolysyl-tRNA synthetase (Mb PylRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-2 variant contains a codon AXC at position 64 of the sequence that encodes the protein having SEQ ID NO: 1, wherein X is an unnatural nucleotide. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

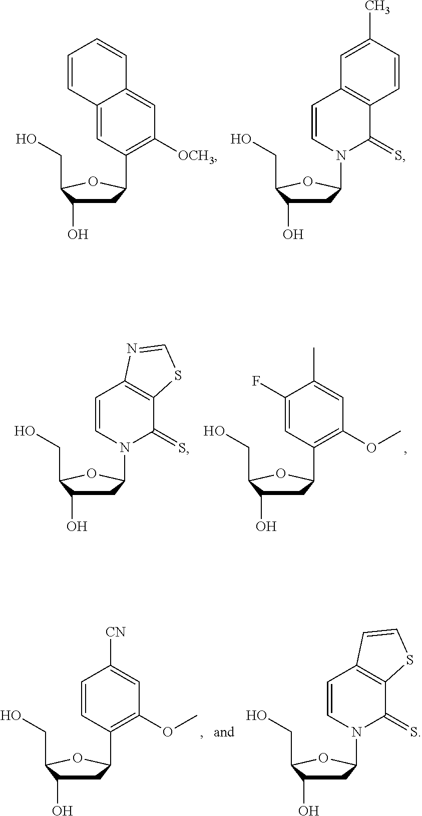

In some embodiments, the first and second unnatural nucleotides comprising the unnatural base pair in the double-stranded oligonucleotide may be derived from

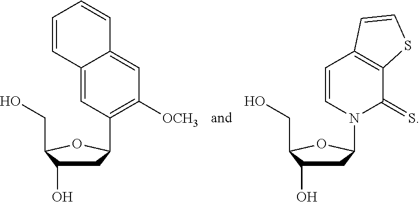

In some embodiments, the triphosphates of the first and second unnatural nucleotides include,

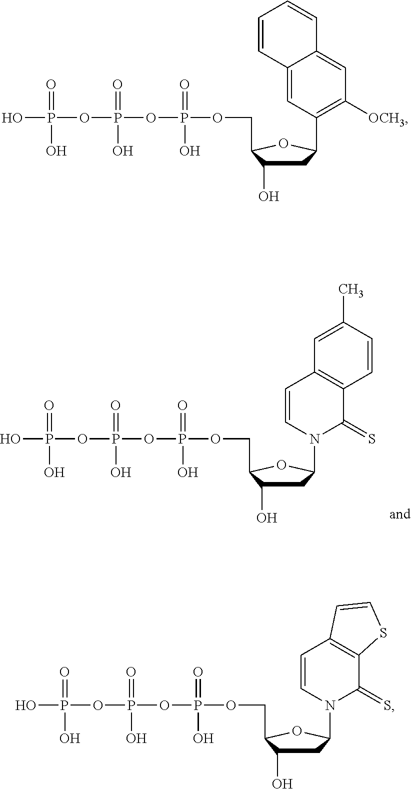

or salts thereof. In some embodiments, the triphosphates of the first and second unnatural nucleotides include,

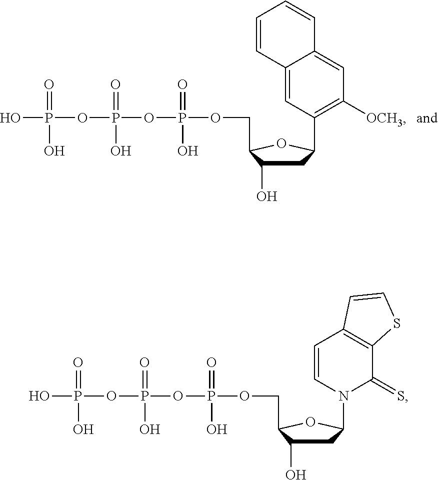

or salts thereof. In some embodiments, the mRNA derived the double-stranded oligonucleotide comprising a first unnatural nucleotide and a second unnatural nucleotide may comprise a codon comprising an unnatural nucleotide derived from

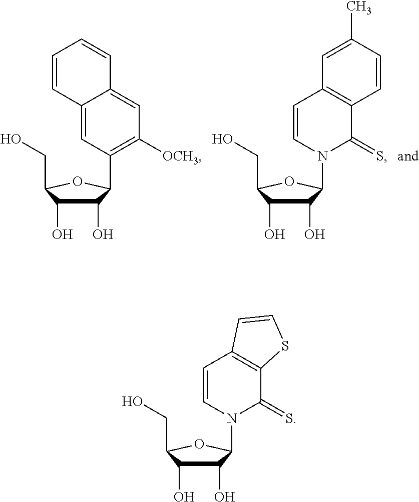

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

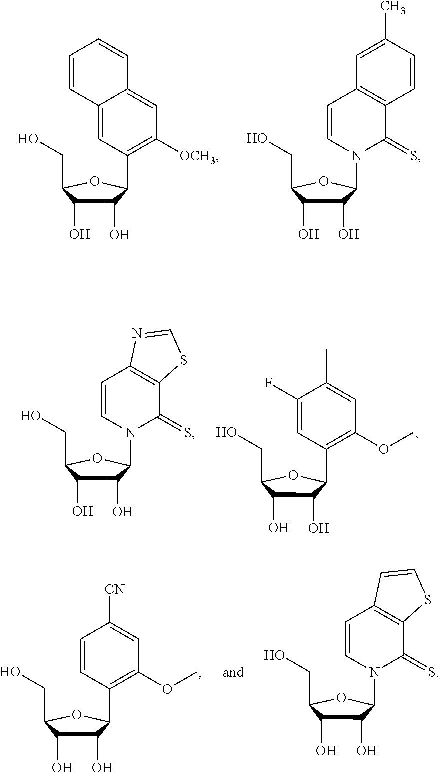

In some embodiments, the mRNA comprises an unnatural nucleotide derived from

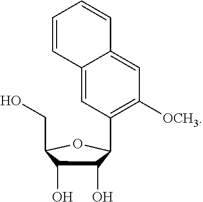

In some embodiments, the mRNA comprises an unnatural nucleotide derived from

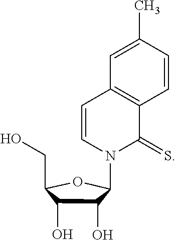

In some embodiments, the mRNA comprises an unnatural nucleotide derived from

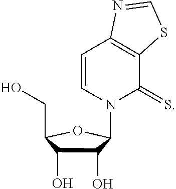

In some embodiments, the mRNA comprises an unnatural nucleotide derived from

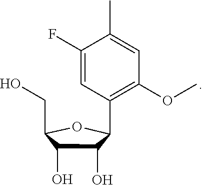

In some embodiments, the mRNA comprises an unnatural nucleotide derived from

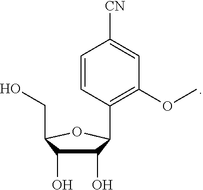

In some embodiments, the mRNA comprises an unnatural nucleotide derived from

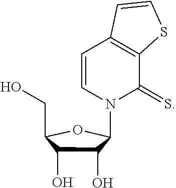

In some embodiments, the tRNA comprises an unnatural nucleotide derived from

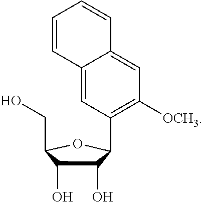

In some embodiments, the tRNA comprises an unnatural nucleotide derived from

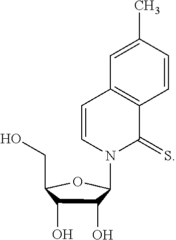

In some embodiments, the tRNA comprises an unnatural nucleotide derived from

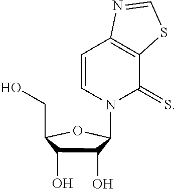

In some embodiments, the tRNA comprises an unnatural nucleotide derived from

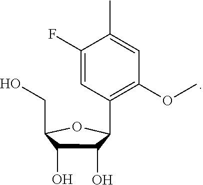

In some embodiments, the tRNA comprises an unnatural nucleotide derived from

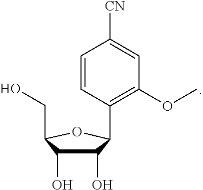

In some embodiments, the tRNA comprises an unnatural nucleotide derived from

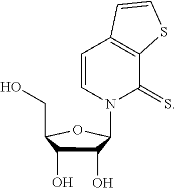

In some embodiments, the mRNA comprises an unnatural nucleotide derived from

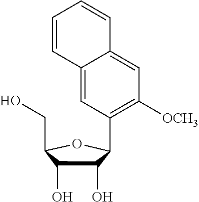

and the tRNA comprises an unnatural nucleotide derived from

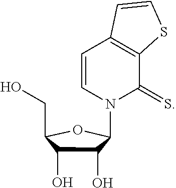

In some embodiments, the mRNA comprises an unnatural nucleotide derived from

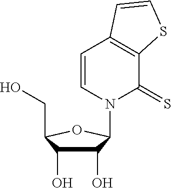

and the tRNA comprises an unnatural nucleotide derived from

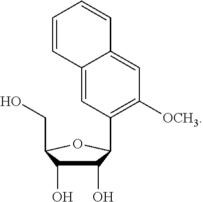

In some embodiments, the mRNA comprises an unnatural nucleotide derived from

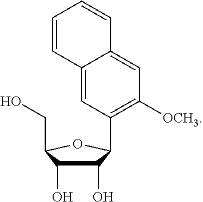

and the tRNA comprises an unnatural nucleotide derived from

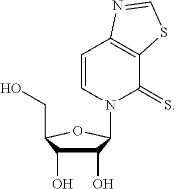

In some embodiments, the mRNA comprises an unnatural nucleotide derived from

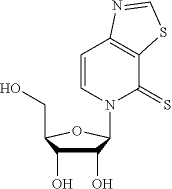

and the tRNA comprises an unnatural nucleotide derived from

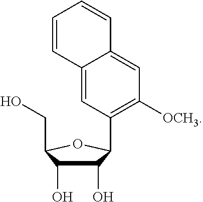

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting AzK-containing protein that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

Alternatively, an IL-2 polypeptide comprising an unnatural amino acid(s) is prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the IL-2 polypeptide. Alternatively, two or more unnatural amino acids may be incorporated into the IL-2 polypeptide at two or more sites in the protein.

Once the IL-2 polypeptide incorporating the unnatural amino acid(s) has been produced in the host cell it can be extracted therefrom by a variety of techniques known in the art, including enzymatic, chemical and/or osmotic lysis and physical disruption. The IL-2 polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21(DE3)), but most suitably host cells are eukaryotic cells, for example insect cells (e.g. Drosophila such as Drosophila melanogaster), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell—such as a human cell or an insect cell. In some embodiments, the suitable host cells comprise E. coli.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### Methods of Treatment

In one aspect, provided herein is a method of treating HNSCC in a subject in need thereof, comprising administering to the subject: (a) an IL-2 conjugate as described herein, and (b) cetuximab.

In a further aspect, provided herein is a method of treating HNSCC in a subject in need thereof, comprising administering to the subject (a) an IL-2 conjugate as described herein, and (b) cetuximab, wherein the HNSCC is recurrent and/or metastatic HNSCC.

In a further aspect, provided herein is a method of treating HNSCC in a subject in need thereof, comprising: selecting a subject having HNSCC, wherein the subject is selected at least in part on the basis of the subject having recurrent and/or metastatic HNSCC; and administering to the subject (a) an IL-2 conjugate as described herein, and (b) cetuximab.

In a further aspect, provided herein is a method of treating HNSCC in a subject in need thereof, comprising administering to the subject (a) an IL-2 conjugate as described herein, and (b) cetuximab, wherein the HNSCC is platinum-refractory HNSCC.

In a further aspect, provided herein is a method of treating HNSCC in a subject in need thereof, comprising: selecting a subject having HNSCC, wherein the subject is selected at least in part on the basis of the subject having platinum-refractory HNSCC; and administering to the subject (a) an IL-2 conjugate as described herein, and (b) cetuximab.

In yet a further aspect, provided herein is a method of treating HNSCC in a subject in need thereof, comprising administering to the subject (a) from 8 μg/kg to 32 μg/kg IL-2 as an IL-2 conjugate as described herein, and (b) cetuximab.

Also provided herein is an IL-2 conjugate as described herein for use in a method disclosed herein of treating HNSCC in a subject in need thereof.

In a further aspect, provided herein is use of an IL-2 conjugate as described herein for the manufacture of a medicament for a method disclosed herein of treating HNSCC in a subject in need thereof.

The embodiments described in the following sections apply to any of the foregoing aspects.

### Cancer Types

In some embodiments, the HNSCC is recurrent and/or metastatic (R/M). In some embodiments, the HNSCC is recurrent. In some embodiments, the HNSCC is metastatic. In some embodiments, the HNSCC is recurrent and metastatic. In some embodiments, the HNSCC is stage III. In some embodiments, the HNSCC is stage IV. In some embodiments, the HNSCC is platinum-refractory HNSCC. In some embodiments, the primary tumor location of the HNSCC is the oropharynx, oral cavity, hypopharynx, or larynx.

### Agents for Combination Therapy

The methods disclosed herein for treatment of HNSCC comprise administering the IL-2 conjugate described herein in combination with one or more additional agents. In some embodiments, the one or more additional agents is cetuximab.

### Administration

### 1. Route

In some embodiments, the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 conjugate is administered to the subject by intravenous, subcutaneous, or intramuscular administration. In some embodiments, the IL-2 conjugate is administered to the subject by intravenous administration. In some embodiments, the IL-2 conjugate is administered to the subject by subcutaneous administration. In some embodiments, the IL-2 conjugate is administered to the subject by intramuscular administration.

In some embodiments, cetuximab is administered by intravenous administration. In some embodiments, the IL-2 conjugate and cetuximab are each administered by intravenous administration.

### 2. Schedule

The IL-2 conjugate may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

Cetuximab may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 conjugate and cetuximab are administered more than once, e.g., twice, three times, four times, five times, or more. In any of these embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 conjugate is administered to a subject in need thereof about once every week, about once every two weeks, about once every three weeks, or about once every 4 weeks. In some embodiments, the IL-2 conjugate is administered to a subject in need thereof once every week. In some embodiments, the IL-2 conjugate is administered to a subject in need thereof once every two weeks. In some embodiments, the IL-2 conjugate is administered to a subject in need thereof once every three weeks. In some embodiments, the IL-2 conjugate is administered to a subject in need thereof once every 4 weeks. In some embodiments, the IL-2 conjugate is administered about once every 7, 14, 15, 16, 17, 18, 19, 20, or 21 days.

In some embodiments, cetuximab is administered to a subject in need thereof about once every week, about once every two weeks, about once every three weeks, or about once every 4 weeks. In some embodiments, cetuximab is administered to a subject in need thereof once every week. In some embodiments, cetuximab is administered to a subject in need thereof once every two weeks. In some embodiments, cetuximab is administered to a subject in need thereof once every three weeks. In some embodiments, cetuximab is administered to a subject in need thereof once every 4 weeks. In some embodiments, cetuximab is administered about once every 7, 14, 15, 16, 17, 18, 19, 20, or 21 days.

In some embodiments, the IL-2 conjugate and cetuximab are administered to a subject in need thereof about once every week, about once every two weeks, about once every three weeks, or about once every 4 weeks. In some embodiments, the IL-2 conjugate and cetuximab are administered to a subject in need thereof once every week. In some embodiments, the IL-2 conjugate and cetuximab are administered to a subject in need thereof once every two weeks. In some embodiments, the IL-2 conjugate and cetuximab are administered to a subject in need thereof once every three weeks. In some embodiments, the IL-2 conjugate and cetuximab are administered to a subject in need thereof once every 4 weeks. In some embodiments, the IL-2 conjugate and cetuximab are administered about once every 7, 14, 15, 16, 17, 18, 19, 20, or 21 days. In some embodiments, the IL-2 conjugate is administered to a subject in need thereof about once every 3 weeks, and cetuximab is administered to the subject about once every week.

In some embodiments, the IL-2 conjugate is administered to the subject separately from the administration of cetuximab. In some embodiments, the IL-2 conjugate and cetuximab are administered to the subject sequentially. In some embodiments, the IL-2 conjugate is administered to the subject prior to the administration to the subject of cetuximab. In some embodiments, the IL-2 conjugate is administered to the subject after the administration to the subject of cetuximab. In some embodiments, the IL-2 conjugate and cetuximab are administered to the subject simultaneously. In some embodiments, the IL-2 conjugate and cetuximab are administered to the subject on the same day. In some embodiments, the IL-2 conjugate and cetuximab are administered to the subject on different days.

### 3. Dosing

In some instances, the desired doses are conveniently presented in a single dose or as divided doses administered simultaneously (or over a short period of time) or at appropriate intervals, for example as two, three, four or more sub-doses per day.

In some embodiments, the IL-2 conjugate is administered at a dose from about 8 μg/kg to 32 μg/kg. In some embodiments, the IL-2 conjugate is administered at a dose from about 8 μg/kg to 24 μg/kg. In some embodiments, the IL-2 conjugate is administered at a dose of about 8 μg/kg. In some embodiments, the IL-2 conjugate is administered at a dose of about 16 μg/kg. In some embodiments, the IL-2 conjugate is administered at a dose of about 24 μg/kg. In some embodiments, the IL-2 conjugate is administered at a dose of about 32 μg/kg. In any of these embodiments, the IL-2 conjugate can be administered at a dose as described herein every 3 weeks.

In some embodiments, the IL-2 conjugate is administered at a dose from about 8 μg/kg to 32 μg/kg in combination with cetuximab. In some embodiments, the IL-2 conjugate is administered at a dose from about 8 μg/kg to 24 μg/kg in combination with cetuximab. In some embodiments, the IL-2 conjugate is administered at a dose of about 8 μg/kg in combination with cetuximab. In some embodiments, the IL-2 conjugate is administered at a dose of about 16 μg/kg in combination with cetuximab. In some embodiments, the IL-2 conjugate is administered at a dose of about 24 μg/kg in combination with cetuximab. In some embodiments, the IL-2 conjugate is administered at a dose of about 32 μg/kg in combination with cetuximab. In any of these embodiments, the IL-2 conjugate can be administered at a dose as described herein every 3 weeks.

In some embodiments, cetuximab is administered at a loading dose from about 100 mg/m² to about 500 mg/m² by intravenous infusion. In any of the embodiments described herein, the loading dose of cetuximab is mg/m² of the subject's body surface area. In some embodiments, cetuximab is administered at a loading dose of about 100 mg/m² by intravenous infusion. In some embodiments, cetuximab is administered at a loading dose of about 150 mg/m² by intravenous infusion. In some embodiments, cetuximab is administered at a loading dose of about 200 mg/m² by intravenous infusion. In some embodiments, cetuximab is administered at a loading dose of about 250 mg/m² by intravenous infusion. In some embodiments, cetuximab is administered at a loading dose of about 300 mg/m² by intravenous infusion. In some embodiments, cetuximab is administered at a loading dose of about 350 mg/m² by intravenous infusion. In some embodiments, cetuximab is administered at a loading dose of about 400 mg/m² by intravenous infusion. In some embodiments, cetuximab is administered at a loading dose of about 450 mg/m² by intravenous infusion. In some embodiments, cetuximab is administered at a loading dose of about 500 mg/m² by intravenous infusion. In some embodiments, the initial dose of cetuximab is administered at a loading dose of about 400 mg/m² by intravenous infusion, and all subsequent doses of cetuximab are administered at a loading dose of about 250 mg/m² by intravenous infusion. In any of these embodiments, cetuximab is infused over about 30-240 minutes. In some embodiments, cetuximab is infused over about 30 minutes. In some embodiments, cetuximab is infused over about 60 minutes. In some embodiments, cetuximab is infused over about 90 minutes. In some embodiments, cetuximab is infused over about 120 minutes. In some embodiments, cetuximab is infused over about 150 minutes. In some embodiments, cetuximab is infused over about 180 minutes. In some embodiments, cetuximab is infused over about 210 minutes. In some embodiments, cetuximab is infused over about 240 minutes. In any of these embodiments, cetixumab is administered at an infusion rate of about 1 mg/min to about 10 mg/min, such as 1 mg/min, 2 mg/min, 3 mg/min, 4 mg/min, 5 mg/min, 6 mg/min, 7 mg/min, 8 mg/min, 9 mg/min, or 10 mg/min. In some embodiments, the first dose of cetuximab is administered at a higher loading dose than the dose of subsequent doses of cetuximab. In some embodiments, the infusion time of the first dose of cetuximab is longer than the infusion time of subsequent doses of cetuximab. In some embodiments, cetuximab is administered at a dose as described herein every 3 weeks. In some embodiments, cetuximab is administered at a dose as described herein every 2 weeks. In some embodiments, cetuximab is administered at a dose as described herein every week.

### 4. Additional Agents/Premedication

In some embodiments, any of the methods described herein further comprises administering an antihistamine. In some embodiments, the antihistamine is cetirizine. In some embodiments, the antihistamine is promethazine. In some embodiments, the antihistamine is dexchlorpheniramine. In some embodiments, the antihistamine is diphenhydramine. In some embodiments, diphenhydramine is administered intravenously at a dose from about 25 to 50 mg.

In some embodiments, any of the methods described herein further comprises administering an analgesic, such as acetaminophen. In some embodiments, acetaminophen is administered orally at a dose from about 650 to 1000 mg.

In some embodiments, any of the methods described herein further comprises administering a serotonin 5-HT3 receptor antagonist. In some embodiments, the serotonin 5-HT3 receptor antagonist is granisetron. In some embodiments, the serotonin 5-HT3 receptor antagonist is dolasetron. In some embodiments, the serotonin 5-HT3 receptor antagonist is tropisetron. In some embodiments, the serotonin 5-HT3 receptor antagonist is palonosetron. In some embodiments, the serotonin 5-HT3 receptor antagonist is ondansetron. In some embodiments, ondansetron is administered intravenously at a dose from about 8 mg to 0.15 mg/kg.

In some embodiments, any of the methods described herein further comprises administering an antihistamine (such as cetirizine, promethazine, dexchlorpheniramine, or diphenhydramine), an analgesic (such as acetaminophen), and/or a serotonin 5-HT3 receptor antagonist (such as granisetron, dolasetron, tropisetron, palonosetron, or ondansetron). In some embodiments, the method further comprises administering an antihistamine (such as cetirizine, promethazine, dexchlorpheniramine, or diphenhydramine) and an analgesic (such as acetaminophen). In some embodiments, the method further comprising administering an antihistamine (such as cetirizine, promethazine, dexchlorpheniramine, or diphenhydramine) and a serotonin 5-HT3 receptor antagonist (such as granisetron, dolasetron, tropisetron, palonosetron, or ondansetron). In some embodiments, the method further comprising administering an analgesic (such as acetaminophen) and a serotonin 5-HT3 receptor antagonist (such as granisetron, dolasetron, tropisetron, palonosetron, or ondansetron). In some embodiments, any of the methods described herein further comprises administering an antihistamine (such as cetirizine, promethazine, dexchlorpheniramine, or diphenhydramine), an analgesic (such as acetaminophen), and a serotonin 5-HT₃ receptor antagonist (such as granisetron, dolasetron, tropisetron, palonosetron, or ondansetron).

In some embodiments, any of the methods described herein further comprises administering a premedication, for example to prevent or reduce the acute effect of infusion-associated reactions (IAR) or flu-like symptoms. In some embodiments, the premedication is administered prior to administering the IL-2 conjugate and/or cetuximab. In some embodiments, the premedication is administered prior to administering the IL-2 conjugate. In some embodiments, the premedication is administered prior to administering cetuximab. In some embodiments, the premedication is administered prior to administering the IL-2 conjugate and cetuximab.

In some embodiments, the premedication for the IL-2 conjugate is different from the premedication for cetuximab. In some embodiments, the premedication for the IL-2 conjugate is the same as the premedication for cetuximab. In some instances where the premedication for the IL-2 conjugate and cetuximab is the same, only a single dose of premedication is administered. In other instances where the premedication for the IL-2 conjugate and cetuximab is the same, multiple doses of premedication are administered. In some embodiments, the premedication is administered for all doses administered of the IL-2 conjugate. In some embodiments, the premedication is administered for the first 1, 2, 3, 4, 5, 6, 7, 8, 9, or 10 doses of the IL-2 conjugate and not for any subsequent doses of the IL-2 conjugate. In some embodiments, the premedication is administered for the first 4 doses of the IL-2 conjugate and not for any subsequent doses of the IL-2 conjugate. In some embodiments, the premedication is administered for all doses administered of cetuximab. In some embodiments, the premedication is administered for the first 1, 2, 3, 4, 5, 6, 7, 8, 9, or 10 doses of cetuximab and not for any subsequent doses of cetuximab. In some embodiments, the premedication is administered for the first dose of cetuximab and not for any subsequent doses of cetuximab.

In some embodiments, any of the methods described herein further comprises administering premedication prior to administering the IL-2 conjugate. In some embodiments, the IL-2 conjugate premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the IL-2 conjugate premedication is a serotonin 5-HT3 receptor antagonist (such as granisetron, dolasetron, tropisetron, palonosetron, or ondansetron). In some embodiments, the serotonin 5-HT3 receptor antagonist is ondansetron. In some embodiments, ondansetron is administered intravenously at a dose from about 8 mg to 0.15 mg/kg. In some embodiments, the IL-2 conjugate premedication is an analgesic (such as acetaminophen). In some embodiments, acetaminophen is administered orally at a dose from about 650 to 1000 mg.

In some embodiments, any of the methods described herein further comprises administering premedication prior to administering cetuximab. In some embodiments, the cetuximab premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the cetuximab premedication is a serotonin 5-HT3 receptor antagonist (such as granisetron, dolasetron, tropisetron, palonosetron, or ondansetron). In some embodiments, the serotonin 5-HT3 receptor antagonist is ondansetron. In some embodiments, ondansetron is administered intravenously at a dose from about 8 mg to 0.15 mg/kg. In some embodiments, the cetuximab premedication is an analgesic (such as acetaminophen). In some embodiments, acetaminophen is administered orally at a dose from about 650 to 1000 mg.

In some embodiments, any of the methods described herein further comprises administering a first dose of premedication prior to administering the IL-2 conjugate and a second dose of premedication prior to administering cetuximab. In some embodiments, the premedication for the IL-2 conjugate is the same as the premedication for cetuximab. In some embodiments, the premedication for the IL-2 conjugate is different from the premedication for cetuximab. In some embodiments, the premedication is an antihistamine, such as cetirizine, promethazine, dexchlorpheniramine, or diphenhydramine. In some embodiments, the antihistamine is diphenhydramine. In some embodiments, diphenhydramine is administered intravenously at a dose from about 25 to 50 mg. In some embodiments, the premedication is a serotonin 5-HT3 receptor antagonist (such as granisetron, dolasetron, tropisetron, palonosetron, or ondansetron). In some embodiments, the serotonin 5-HT3 receptor antagonist is ondansetron. In some embodiments, ondansetron is administered intravenously at a dose from about 8 mg to 0.15 mg/kg. In some embodiments, the premedication is an analgesic (such as acetaminophen). In some embodiments, acetaminophen is administered orally at a dose from about 650 to 1000 mg. In some embodiments, the premedication comprises an antihistamine and a serotonin 5-HT3 receptor antagonist. In some embodiments, the premedication comprises an antihistamine and an analgesic. In some embodiments, the premedication comprises a serotonin 5-HT3 receptor antagonist and an analgesic. In some embodiments, the premedication comprises an antihistamine, a serotonin 5-HT3 receptor antagonist, and an analgesic. In some instances where the premedication for the IL-2 conjugate and cetuximab is the same (such as diphenhydramine), only a single dose of premedication is administered. In other instances where the premedication for the IL-2 conjugate and cetuximab is the same, multiple doses of premedication are administered.

### 5. Dosing Sequence

In some embodiments, the premedication for the IL-2 conjugate and/or cetuximab is as described above and is administered as part of a dosing sequence comprising administering the IL-2 conjugate.

In some embodiments of the methods described herein, the dosing sequence is as follows: (i) premedication for cetuximab; (ii) cetuximab; (iii) premedication for the IL-2 conjugate; and (iv) IL-2 conjugate. In some variations where the premedication for cetuximab is the same as the premedication for the IL-2 conjugate (such as diphenhydramine), administering the premedication for the IL-2 conjugate may be omitted.

In some embodiments, the premedication for the IL-2 conjugate is administered about 30-60 minutes prior to administering the IL-2 conjugate, for example, 30-60 minutes prior to the start of the IL-2 conjugate infusion. In some embodiments, the premedication for cetuximab is administered about 30-60 minutes prior to administering cetuximab, for example, 30-60 minutes prior to the start of cetuximab infusion.

### Subject

In some embodiments, administration of the IL-2 conjugate and the one or more additional agents is to an adult. In some embodiments, the adult is a male. In other embodiments, the adult is a female. In some embodiments, the adult is at least age 18, 19, 20, 25, 30, 35, 40, 45, 50, 55, 60, 65, 70, 75, 80, 85, 90, or 95 years of age.

In some embodiments, the subject has measurable disease (i.e., HNSCC). Measureable disease may be determined by RECIST v1.1. For example, the subject may have at least one measurable lesion per RECIST v1.1. In some embodiments, the subject has histologically or cytologically confirmed diagnosis of recurrent and/or metastatic (R/M) HNSCC that is not amenable to further therapy with curative intent. In some embodiments, the primary tumor location of the HNSCC in the subject is oropharynx, oral cavity, hypopharynx, or larynx. In some embodiments, the primary tumor location is not the nasopharynx. In some embodiments, the subject HPV p16 status for oropharyngeal cancer is known. In some embodiments, the subject has been determined to have Eastern Cooperative Oncology Group (ECOG) performance status of <2, e.g., 0 or 1. In some embodiments, the subject has adequate cardiovascular, hematological, liver, and renal function, as determined by a physician. In some embodiments, the subject has been determined (e.g., by a physician) to have a life expectancy greater than or equal to 12 weeks. In some embodiments, the subject has had prior anti-cancer therapy before administration of the first treatment dose. In some embodiments, the subject has a histologically or cytologically confirmed diagnosis of R/M HNSCC that is considered not amenable to further therapy with curative intent. In some embodiments, if a subject has oropharyngeal cancer, then the subject has a known human papillomavirus p16 status. In some embodiments, the subject does not have a history of allogenic tissue/solid organ transplant. In some embodiments, the subject did not experience an immune-mediated/related toxicity from prior immunooncology therapy of Grade 4 or leading to discontinuation. In some embodiments, the subject does not have ongoing AEs caused by any prior anti-cancer therapy >Grade 2. In some embodiments, the subject does not have baseline oxygen saturation (SpO2)<92% (without oxygen therapy). In some embodiments, the subject has not received prior IL2-based anticancer treatment. In some embodiments, the subject can temporarily (for at least 36 hours) withhold any antihypertensive medications prior to each dose of the IL-2 conjugate. In some embodiments, for methods in which the therapy comprises administering cetuximab, the subject did not receive prior treatment with cetuximab. In some embodiments, the subject does not have electrolyte (magnesium, calcium, and potassium) levels outside of normal ranges. In some embodiments, a subject meets each of the foregoing criteria.

In some embodiments, the subject is treatment-naïve for R/M HNSCC. In some embodiments, the subject was not previously treated with cetuximab (i.e., the patient is treatment-naïve for cetuximab).

In some embodiments, the subject was previously treated with a platinum-based regimen. In some embodiments, the subject has platinum-refractory HNSCC. In some embodiments, the subject's previous treatment for HNSCC comprised failure of no more than two regimens. In some embodiments, the subject's previous treatment for HNSCC comprised failure of one regimen. In some embodiments, the subject's previous treatment for HNSCC comprised failure of two regimens. In some embodiments, the subject's previous treatment for HNSCC comprised failure of no more than two regimens, wherein at least one of the failed regimens was a platinum-based regimen. In some embodiments, the subject's previous treatment for HNSCC comprised failure of a checkpoint-based regimen. In some embodiments, the subject's previous treatment for HNSCC comprised failure of a checkpoint-based regimen and a platinum-based regimen. In some embodiments, the subject has platinum-refractory HNSCC and the subject's previous treatment for HNSCC comprised failure of no more than two regimens. In some embodiments, the subject has platinum-refractory HNSCC and the subject's previous treatment for HNSCC comprised failure of one regimen. In some embodiments, the subject has platinum-refractory HNSCC and the subject's previous treatment for HNSCC comprised failure of two regimens. In some embodiments, the subject is a 1 L R/M HNSCC subject. In some embodiments, the subject is a 2/3L R/M HNSCC subject.

In some embodiments, the subject has no known hypersensitivity or contraindications to any of the IL-2 conjugates disclosed herein, PEG, pegylated drugs, or cetuximab. In some embodiments, the subject has not received a previous anticancer treatment comprising IL-2. In some embodiments, the subject has not received a previous anticancer treatment comprising cetuximab.

In some embodiments, the subject does not have an Eastern Cooperative Oncology Group (ECOG) performance status of greater than or equal to 2. In some embodiments, the subject does not have a predicted life expectancy less than or equal to 3 months.

In some embodiments, the subject does not have active brain metastases or leptomeningeal metastases. In some embodiments, the subject was previously treated for brain metastases, has been clinically stable for at least 4 weeks prior to administration of the IL-2 conjugate combination therapy, has no evidence of new or enlarging brain metastases, and has not received steroids for at least 2 weeks prior to administration of the IL-2 conjugate combination therapy. In some embodiments, the subject has asymptomatic brain metastases (i.e., no neurological symptoms, no requirements for corticosteroids, and no lesion greater than 1.5 cm) and receives regular imaging of the brain as a site of disease.

In some embodiments, the subject has no history of allogenic or solid organ transplant.

In some embodiments, the subject does not have treatment-related immune-mediated (or immune-related) adverse events (AEs) from immune-modulatory agents (including, but not limited to anti-cytotoxic T lymphocyte associated protein 4 monoclonal antibodies) that caused permanent discontinuation of the agent, or that were Grade 4 in severity.

In some embodiments, the subject's last administration of prior antitumor therapy (chemotherapy, targeted agents, and immunotherapy) or any investigational treatment was not within 28 days or less than 5 times the half-life, whichever is shorter, prior to administration of the IL-2 conjugate combination therapy. In some embodiments, the subject did not have major surgery or local intervention within 28 days of receiving the IL-2 combination therapy.

In some embodiments, the subject does not have comorbidity requiring corticosteroid therapy (>10 mg prednisone/day or equivalent) within 2 weeks of receiving the first dose of the IL-2 conjugate combination therapy. In some embodiments, the subject receives inhaled or topical steroids, provided that they are not for treatment of an autoimmune disorder. In some embodiments, the subject receives a brief course of steroids (e.g., as prophylaxis for imaging studies due to hypersensitivity to contrast agents).

In some embodiments, the subject has not received antibiotics (excluding topical antibiotics) within 14 days of receiving the first dose of the IL-2 conjugate combination therapy. In some embodiments, the subject does not have any serious systemic fungal, bacterial, viral, or other infections that are not controlled or require IV or oral antibiotics.

In some embodiments, the subject has not had a severe or unstable cardiac condition within 6 months of administration of the IL-2 conjugate combination therapy, such as congestive heart failure (New York Heart Association Class III or IV), cardiac bypass surgery or coronary artery stent placement, angioplasty, left ventricular ejection fraction (LVEF) below 50%, unstable angina, medically uncontrolled hypertension (e.g., ≥160 mmHg systolic or ≥100 mmHg diastolic), uncontrolled cardiac arrhythmia requiring medication (≥Grade 2, according to NCI-CTCAE v5.0), or myocardial infarction. In some embodiments, the subject does not have significant valvular heart disease (including valve replacement), vascular malformation, and aneurysm.

In some embodiments, the subject does not have ongoing AEs caused by a prior anticancer therapy ≥Grade 2 (NCI-CTCAE Version 5.0). In some embodiments, the subject has Grade 2 peripheral neuropathy or Grade 2 alopecia.

In some embodiments, the subject has not had active, known, or suspected autoimmune disease that has required systemic treatment (i.e., use of disease modifying agents, corticosteroids, or immunosuppressive drugs) within 2 years of administering the IL-2 conjugate combination therapy. In some embodiments, the subject has received replacement therapy for an autoimmune disease (e.g., thyroxine, insulin, or physiologic corticosteroid replacement therapy for adrenal or pituitary insufficiency, etc). In some embodiments, the subject has had vitiligo, childhood asthma that has resolved, or psoriasis that does not require systemic treatment.

In some embodiments, the subject does not have pneumonitis or interstitial lung disease, or a history of interstitial lung disease or pneumonitis that required oral or IV glucocorticoids to assist with management.

In some embodiments, the subject has not received radiotherapy within 2 weeks of receiving the first dose of the IL-2 conjugate combination therapy. In some embodiments, the subject has recovered from all radiation-related toxicities, does not require corticosteroids, and did not have radiation pneumonitis. In some embodiments, the subject has had a one-week washout for palliative radiation (≤2 weeks of radiotherapy) relating to non-CNS disease.

In some embodiments, the subject did not receive a live-virus vaccination within 28 days of receiving the first dose of the IL-2 conjugate combination therapy.

In some embodiments, the subject is not HIV-infected with a history of Kaposi sarcoma and/or Multicentric Castleman Disease or known uncontrolled infection with HIV. In some embodiments, the subject is HIV-infected and is on anti-retroviral therapy (ART) and has a well-controlled HIV infection/disease defined as: subjects on ART have a CD4+ T-cell count >350 cells/mm³; subjects on ART have achieved and maintained virologic suppression defined as confirmed HIV RNA level below 50 copies/mL or the lower limit of qualification (below the limit of detection) using a locally available assay and for at least 12 weeks; subjects on ART are on a stable regimen, without changes in drugs or dose modification, for at least 4 weeks prior to receiving the first dose of the IL-2 conjugate combination therapy; combination ART regimen does not contain any antiretroviral medications other than abacavir, dolutegravir, emtricitabine, lamivudine, raltegravir, rilpivirine, or tenoforvir.

In some embodiments, the subject does not have known uncontrolled hepatitis B infection, known untreated hepatitis C infection, active tuberculosis, or severe infection requiring parenteral antibiotic treatment. In some embodiments, the subject has positive HBsAg and has started anti-HBV therapy to control HBV infection prior to receiving the first dose of the IL-2 conjugate combination therapy. In some embodiments, the subject has received antiviral therapy for HBV for at least 4 weeks and has an HBV viral load of less than 100 IU/mL prior to receiving the first dose of the IL-2 conjugate combination therapy. In some embodiments, the subject has a viral load under 100 IU/mL and receives active HBV therapy throughout the IL-2 conjugate combination therapy.

In some embodiments, the subject is positive for anti-hepatitis B core antibody HBc, negative for hepatitis B surface antigen (HBsAg), negative or positive for anti-hepatitis B surface antibody (HBs), has an HBV viral load under 100 IU/mL, and does not require HBV anti-viral prophylaxis.

In some embodiments, the subject has past or ongoing HCV infection and has completed treatment at least 1 month prior to receiving the first dose of the IL-2 conjugate combination therapy. In some embodiments, the subject has positive HCV antibody and undetectable HCV RNA and does not receive anti-HCV therapy.

In some embodiments, the subject does not have a known second malignancy either progressing or requiring active treatment within 3 years prior to administering the IL-2 conjugate combination therapy. In some embodiments, the subject has basal cell carcinoma of the skin, squamous cell carcinoma of the skin, or carcinoma in situ (e.g., breast carcinoma, cervical cancer in situ) and has undergone potentially curative therapy.

In some embodiments, the subject does not have underlying cancer predisposition syndromes including, but not limited to, history of hereditary breast and ovarian cancer syndrome, Ferguson-Smith syndrome, multiple self-healing epithelioma, familial adenomatous polyposis, multiple endocrine neoplasia, or Li-Fraumeni syndrome.

In some embodiments, the subject does not have electrolytes (magnesium, calcium, potassium) outside the normal ranges. In some embodiments, the subject does not have baseline SpO₂≤92% (without oxygen therapy).

In some embodiments, the subject has not received prior IL-2 based anticancer treatment. In some embodiments, the subject is able and willing to take premedication. In some embodiments, the subject is not receiving hepatically metabolized narrow therapeutic index drugs (e.g., digoxin, warfarin) without close monitoring. In some embodiments, the subject is receiving anti-hypertensive treatment and the antihypertensive medication is temporarily withheld (for at least 36 hours) prior to receiving each dose of the IL-2 conjugate combination therapy.

In some embodiments, the subject is not being treated with therapeutic doses of anticoagulants or antiplatelet agents (e.g., 1 mg/kg bid of enoxaparin, 300 mg of aspirin daily, 300 mg of clopidogrel daily or equivalent) within 7 days prior to receiving the first dose of the anti-TGFβ antibody. In some embodiments, the subject receives prophylactic treatment of anticoagulants.

In some embodiments, the subject has not received prior treatment with cetuximab unless used locally for the treatment of locally advanced disease, with no progressive disease for at least 4 months from completion of prior cetuximab therapy.

In some embodiments, the subject is not participating in a clinical study concurrently with receiving the IL-2 conjugate combination therapy.

In some embodiments, the subject does not have any or more of the following: absolute neutrophil count <1500/uL (1.5×10⁹/L) (after at least one week off G-CSF); platelets <100×103 u/L (after at least 3 days without platelet transfusion); hemoglobin <9 g/dL (without packed red blood cell [pRBC] transfusion within prior 2 weeks; subjects can be on stable dose of erythropoietin (≥approximately 3 months); total bilirubin >1.5×upper limit of normal (ULN) unless direct bilirubin ≤ULN (subjects with known Gilbert disease who have serum bilirubin level ≤3×ULN are not excluded); aspartate aminotransferase and/or alanine aminotransferase >2.5×ULN (or >5×ULN for subjects with liver metastases); estimated glomerular filtration rate (eGFR)<50 mL/min/1.73 m² (Modification of Diet in Renal Disease [MDRD] Formula); International Normalized Ratio (INR) or Prothrombin Time (PT) or Activated Partial Thromboplastin Time (aPTT) >1.5×ULN unless the subject is receiving anticoagulant therapy as long as PT or aPTT is within therapeutic range of intended use of anticoagulants.

### Effects of Administration

In some embodiments, administration of the IL-2 conjugate combination therapy as described herein provides a complete response, a partial response, or stable disease.

In some embodiments, following administration of the IL-2 conjugate combination therapy, the subject experiences a response as measured by the Immune-related Response Evaluation Criteria in Solid Tumors (iRECIST). In some embodiments, following administration of the IL-2 conjugate combination therapy, the subject experiences an Objective Response Rate (ORR) according to RECIST version 1.1. In some embodiments, following administration of the IL-2 conjugate combination therapy, the subject experiences Duration of Response (DOR) according to RECIST versions 1.1. In some embodiments, following administration of the IL-2 conjugate combination therapy, the subject experiences Progression-Free Survival (PFS) according to RECIST version 1.1. In some embodiments, following administration of the IL-2 conjugate combination therapy, the subject experiences Overall Survival according to RECIST version 1.1. In some embodiments, following administration of the IL-2 conjugate combination therapy, the subject experiences Time to Response (TTR) according to RECIST version 1.1. In some embodiments, following administration of the IL-2 conjugate combination therapy, the subject experiences Disease Control Rate (DCR) according to RECIST version 1.1. In any of these embodiments, the subject's experience is based on a physician's review of a radiographic image taken of the subject.

In some embodiments, administration of the IL-2 conjugate combination therapy to the subject does not cause vascular leak syndrome in the subject. In some embodiments, administration of the IL-2 conjugate combination therapy to the subject does not cause Grade 2, Grade 3, or Grade 4 vascular leak syndrome in the subject. In some embodiments, administration of the IL-2 conjugate combination therapy to the subject does not cause Grade 2 vascular leak syndrome in the subject. In some embodiments, administration of the IL-2 conjugate combination therapy to the subject does not cause Grade 3 vascular leak syndrome in the subject. In some embodiments, administration of the IL-2 conjugate combination therapy to the subject does not cause Grade 4 vascular leak syndrome in the subject. In some embodiments, administration of the IL-2 conjugate combination therapy to the subject does not cause loss of vascular tone in the subject.

In some embodiments, administration of the IL-2 conjugate combination therapy to the subject does not cause extravasation of plasma proteins and fluid into the extravascular space in the subject.

In some embodiments, administration of the IL-2 conjugate combination therapy to the subject does not cause hypotension and reduced organ perfusion in the subject.

In some embodiments, administration of the IL-2 conjugate combination therapy to the subject does not cause impaired neutrophil function in the subject. In some embodiments, administration of the IL-2 conjugate combination therapy to the subject does not cause reduced chemotaxis in the subject.

In some embodiments, administration of the IL-2 conjugate combination therapy to the subject is not associated with an increased risk of disseminated infection in the subject. In some embodiments, the disseminated infection is sepsis or bacterial endocarditis. In some embodiments, the disseminated infection is sepsis. In some embodiments, the disseminated infection is bacterial endocarditis. In some embodiments, the subject is treated for any preexisting bacterial infections prior to administration of the IL-2 conjugate combination therapy. In some embodiments, the subject is treated with an antibacterial agent selected from oxacillin, nafcillin, ciprofloxacin, and vancomycin prior to administration of the IL-2 conjugate combination therapy.

In some embodiments, administration of the IL-2 conjugate combination therapy to the subject does not exacerbate a pre-existing or initial presentation of an autoimmune disease or an inflammatory disorder in the subject. In some embodiments, the administration of the IL-2 conjugate combination therapy to the subject does not exacerbate a pre-existing or initial presentation of an autoimmune disease in the subject. In some embodiments, the administration of the IL-2 conjugate combination therapy to the subject does not exacerbate a pre-existing or initial presentation of an inflammatory disorder in the subject. In some embodiments, the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid. In some embodiments, the autoimmune disease or inflammatory disorder in the subject is Crohn's disease. In some embodiments, the autoimmune disease or inflammatory disorder in the subject is scleroderma. In some embodiments, the autoimmune disease or inflammatory disorder in the subject is thyroiditis. In some embodiments, the autoimmune disease or inflammatory disorder in the subject is inflammatory arthritis. In some embodiments, the autoimmune disease or inflammatory disorder in the subject is diabetes mellitus. In some embodiments, the autoimmune disease or inflammatory disorder in the subject is oculo-bulbar myasthenia gravis. In some embodiments, the autoimmune disease or inflammatory disorder in the subject is crescentic IgA glomerulonephritis. In some embodiments, the autoimmune disease or inflammatory disorder in the subject is cholecystitis. In some embodiments, the autoimmune disease or inflammatory disorder in the subject is cerebral vasculitis. In some embodiments, the autoimmune disease or inflammatory disorder in the subject is Stevens-Johnson syndrome. In some embodiments, the autoimmune disease or inflammatory disorder in the subject is bullous pemphigoid.

In some embodiments, administration of the IL-2 conjugate combination therapy to the subject does not cause changes in mental status, speech difficulties, cortical blindness, limb or gait ataxia, hallucinations, agitation, obtundation, or coma in the subject. In some embodiments, administration of the IL-2 conjugate combination therapy to the subject does not cause seizures in the subject. In some embodiments, administration of the IL-2 conjugate combination therapy to the subject is not contraindicated in subjects having a known seizure disorder.

In some embodiments, administration of the IL-2 conjugate combination therapy to the subject does not cause capillary leak syndrome in the subject. In some embodiments, administration of the IL-2 conjugate combination therapy to the subject does not cause Grade 2, Grade 3, or Grade 4 capillary leak syndrome in the subject. In some embodiments, administration of the IL-2 conjugate combination therapy to the subject does not cause Grade 2 capillary leak syndrome in the subject. In some embodiments, administration of the IL-2 conjugate combination therapy to the subject does not cause Grade 3 capillary leak syndrome in the subject. In some embodiments, administration of the IL-2 conjugate combination therapy to the subject does not cause Grade 4 capillary leak syndrome in the subject.

In some embodiments, administration of the IL-2 conjugate combination therapy to the subject does not cause a drop in mean arterial blood pressure in the subject following administration. In some embodiments, administration of the IL-2 conjugate combination therapy to the subject does cause hypotension in the subject. In some embodiments, administration of the IL-2 conjugate combination therapy to the subject does not cause the subject to experience a systolic blood pressure below 90 mm Hg or a 20 mm Hg drop from baseline systolic pressure.

In some embodiments, administration of the IL-2 conjugate combination therapy to the subject does not cause edema or impairment of kidney or liver function in the subject.

In some embodiments, administration of the IL-2 conjugate combination therapy to the subject does not cause eosinophilia in the subject. In some embodiments, administration of the IL-2 conjugate combination therapy to the subject does not cause the eosinophil count in the peripheral blood of the subject to exceed 500 per μL. In some embodiments, administration of the IL-2 conjugate combination therapy to the subject does not cause the eosinophil count in the peripheral blood of the subject to exceed 500 μL to 1500 per μL. In some embodiments, administration of the the IL-2 conjugate combination therapy to the subject does not cause the eosinophil count in the peripheral blood of the subject to exceed 1500 per μL to 5000 per μL. In some embodiments, administration of the IL-2 conjugate combination therapy to the subject does not cause the eosinophil count in the peripheral blood of the subject to exceed 5000 per μL. In some embodiments, administration of the IL-2 conjugate combination therapy to the subject is not contraindicated in subjects on an existing regimen of psychotropic drugs.

In some embodiments, administration of the IL-2 conjugate combination therapy to the subject is not contraindicated in subjects on an existing regimen of nephrotoxic, myelotoxic, cardiotoxic, or hepatotoxic drugs. In some embodiments, administration of the IL-2 conjugate combination therapy to the subject is not contraindicated in subjects on an existing regimen of aminoglycosides, cytotoxic chemotherapy, doxorubicin, methotrexate, or asparaginase. In some embodiments, administration of the IL-2 conjugate combination therapy to the subject is not contraindicated in subjects receiving combination regimens containing antineoplastic agents. In some embodiments, the antineoplastic agent is selected from dacarbazine, cis-platinum, tamoxifen and interferon-alpha.

In some embodiments, administration of the IL-2 conjugate combination therapy to the subject does not cause one or more Grade 4 adverse events in the subject following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis. In some embodiments, administration of the IL-2 conjugate combination therapy to a group of subjects does not cause one or more Grade 4 adverse events in greater than 1% of the subjects following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

In some embodiments, administration of the IL-2 conjugate combination therapy to a group of subjects does not cause one or more adverse events in greater than 1% of the subjects following administration, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

In some embodiments, administration of the IL-2 conjugate combination therapy to a group of subjects does not cause one or more adverse events in greater than 1% of the subjects following administration, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

In some embodiments, administration of the IL-2 conjugate combination therapy to the subject stimulates CD8+ cells in a subject. In some embodiments, administration of the IL-2 conjugate combination therapy to the subject stimulates NK cells in a subject. Stimulation may comprise an increase in the number of CD8+ cells in the subject, e.g., about 4, 5, 6, or 7 days after administration, or about 1, 2, 3, or 4 weeks after administration. In some embodiments, the CD8+ cells comprise memory CD8+ cells. In some embodiments, the CD8+ cells comprise effector CD8+ cells. Stimulation may comprise an increase in the proportion of CD8+ cells that are Ki67 positive in the subject, e.g., about 4, 5, 6, or 7 days after administration, or about 1, 2, 3, or 4 weeks after administration. Stimulation may comprise an increase in the number of NK cells in the subject, e.g., about 4, 5, 6, or 7 days after administration, or about 1, 2, 3, or 4 weeks after administration.

In some embodiments, CD8+ cells are expanded in the subject following administration of the IL-2 conjugate combination therapy by at least 1.5-fold, such as by at least 1.6-fold, 1.7-fold, 1.8-fold, or 1.9-fold. In some embodiments, NK cells are expanded in the subject following administration of the IL-2 conjugate combination therapy by at least 5-fold, such as by at least 5.5-fold, 6-fold, or 6.5-fold. In some embodiments, eosinophils are expanded in the subject following administration of the IL-2 conjugate combination therapy by no more than about 2-fold, such as no more than about 1.5-fold, 1.4-fold, or 1.3-fold. In some embodiments, CD4+ cells are expanded in the subject following administration of the IL-2 conjugate combination therapy by no more than about 2-fold, such as no more than about 1.8-fold, 1.7-fold, or 1.6-fold. In some embodiments, the expansion of CD8+ cells and/or NK cells in the subject following administration of the IL-2 conjugate combination therapy is greater than the expansion of CD4+ cells and/or eosinophils. In some embodiments, the expansion of CD8+ cells is greater than the expansion of CD4+ cells. In some embodiments, the expansion of NK cells is greater than the expansion of CD4+ cells. In some embodiments, the expansion of CD8+ cells is greater than the expansion of eosinophils. In some embodiments, the expansion of NK cells is greater than the expansion of eosinophils. Fold expansion is determined relative to a baseline value measured before administration of the IL-2 conjugate. In some embodiments, fold expansion is determined at any of the times after administration, such as about 4, 5, 6, or 7 days after administration, or about 1, 2, 3, or 4 weeks after administration.

In some embodiments, administration of the IL-2 conjugate combination therapy to the subject increases the number of peripheral CD8+T and NK cells in the subject without increasing the number of peripheral CD4+ regulatory T cells in the subject. In some embodiments, administration of the IL-2 conjugate combination therapy to the subject increases the number of peripheral CD8+T and NK cells in the subject without increasing the number of peripheral eosinophils in the subject. In some embodiments, administration of the IL-2 conjugate combination therapy to the subject increases the number of peripheral CD8+T and NK cells in the subject without increasing the number of intratumoral CD8+T and NK cells in the subject and without increasing the number of intratumoral CD4+ regulatory T cells in the subject.

In some embodiments, administration of the IL-2 conjugate combination therapy to the subject does not require the availability of an intensive care facility or skilled specialists in cardiopulmonary or intensive care medicine. In some embodiments, administration of the IL-2 conjugate combination therapy to the subject does not require the availability of an intensive care facility or skilled specialists in cardiopulmonary or intensive care medicine. In some embodiments, administration of the IL-2 conjugate combination therapy to the subject does not require the availability of an intensive care facility. In some embodiments, administration of the IL-2 conjugate combination therapy to the subject does not require the availability of skilled specialists in cardiopulmonary or intensive care medicine.

In some embodiments, administration of the IL-2 conjugate combination therapy does not cause dose-limiting toxicity. In some embodiments, administration of the IL-2 conjugate combination therapy does not cause severe cytokine release syndrome. In some embodiments, the IL-2 conjugate does not induce anti-drug antibodies (ADAs), i.e., antibodies against the IL-2 conjugate. In some embodiments, administration of cetuximab does not induce anti-drug antibodies (ADAs), i.e., antibodies against cetuximab. In some embodiments, a lack of induction of ADAs is determined by direct immunoassay for antibodies against PEG and/or ELISA for antibodies against the IL-2 conjugate or cetuximab. An IL-2 conjugate or cetuximab is considered not to induce ADAs if a measured level of ADAs is statistically indistinguishable from a baseline (pre-treatment) level or from a level in an untreated control.

In some embodiments, administration of the IL-2 conjugate combination therapy improves an ADCC response to the HNSCC. In some embodiments, administration of the IL-2 conjugate combination therapy promotes immune activation within the tumor microenvironment. In some embodiments, administration of the IL-2 conjugate combination therapy overcomes or reduces immune evasion mechanisms and boosts anti-cancer T cell immunity. In some embodiments, administration of the IL-2 conjugate combination therapy inhibits the mechanism responsible for resistance of a tumor, for example, EGFR activity.

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. Preparation of Pegylated IL-2 Conjugates

An exemplary method with details for preparing IL-2 conjugates described herein is provided in this Example.

IL-2 employed for bioconjugation was expressed as inclusion bodies in E. coli using methods disclosed herein, using: (a) an expression plasmid encoding (i) the protein with the desired amino acid sequence, which gene contains a first unnatural base pair to provide a codon at the desired position at which an unnatural amino acid N6-((2-azidoethoxy)-carbonyl)-L-lysine (AzK) was incorporated and (ii) a tRNA derived from M. mazei Pyl, which gene comprises a second unnatural nucleotide to provide a matching anticodon in place of its native sequence; (b) a plasmid encoding a M. barkeri derived pyrrolysyl-tRNA synthetase (Mb PylRS), (c) N6-((2-azidoethoxy)-carbonyl)-L-lysine (AzK); and (d) a truncated variant of nucleotide triphosphate transporter PtNTT2 in which the first 65 amino acid residues of the full-length protein were deleted. The double-stranded oligonucleotide that encodes the amino acid sequence of the desired IL-2 variant contained a codon AXC as codon 64 of the sequence that encodes the protein having SEQ ID NO: 1 in which P64 is replaced with an unnatural amino acid described herein. The plasmid encoding an orthogonal tRNA gene from M. mazei comprised an AXC-matching anticodon GYT in place of its native sequence, wherein Y is an unnatural nucleotide as disclosed herein. X and Y were selected from unnatural nucleotides dTPT3 and dNaM as disclosed herein. The expressed protein was extracted from inclusion bodies and re-folded using standard procedures before site-specifically pegylating the AzK-containing IL-2 product using DBCO-mediated copper-free click chemistry to attach stable, covalent mPEG moieties to the AzK. Exemplary reactions are shown in Schemes 1 and 2 (wherein n indicates the number of repeating PEG units). The reaction of the AzK moiety with the DBCO alkynyl moiety may afford one regioisomeric product or a mixture of regioisomeric products.

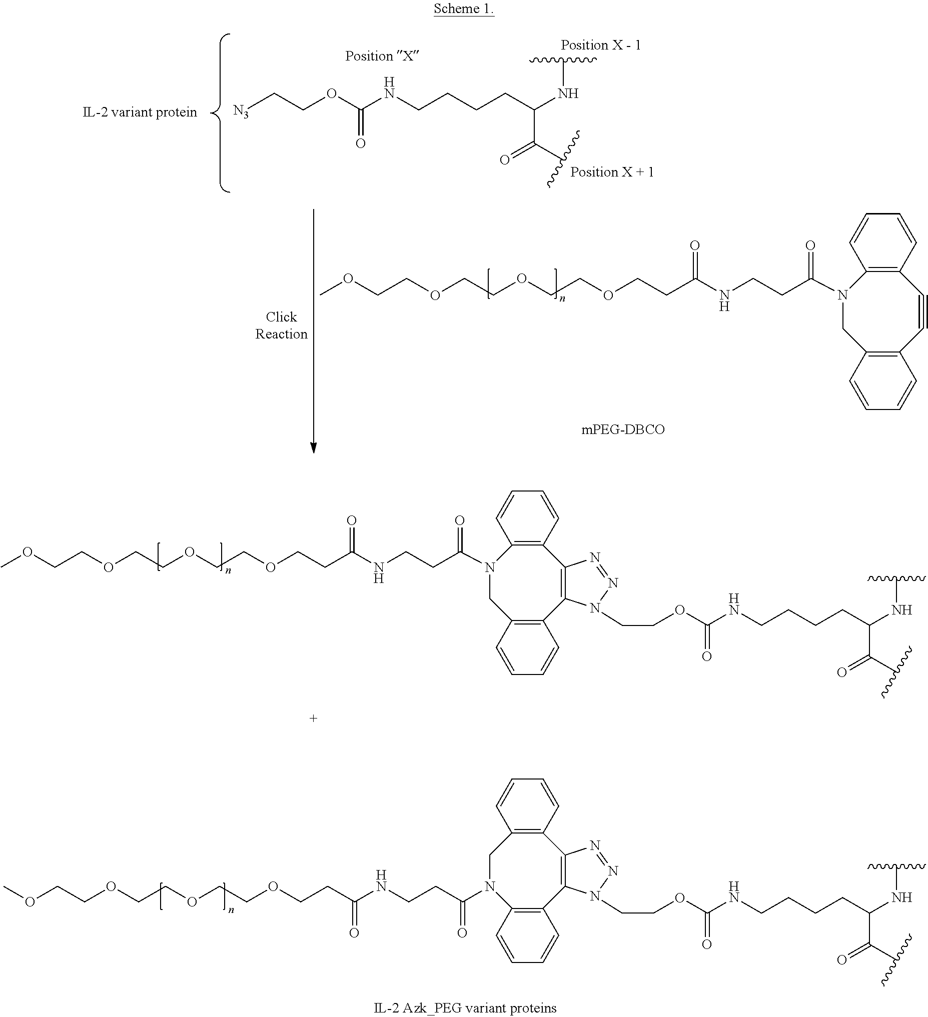

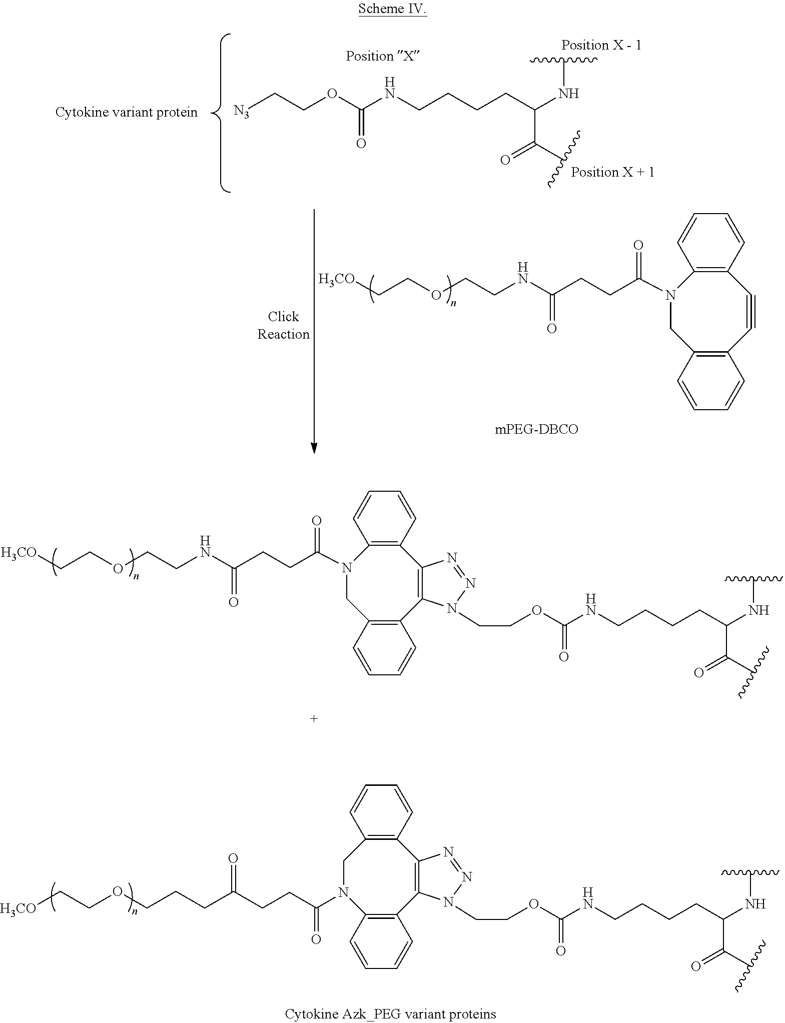

### Example 2. Clinical Study of Combination Therapy Using an IL-2 Conjugate and Cetuximab in Subjects Having Platinum-Refractory HNSCC (Cohort B2)

A Phase 2 non-randomized, open-label, multi-cohort, multi-center study assessing the clinical benefit of an IL-2 conjugate in combination with cetuximab for the treatment of participants with HNSCC was undertaken. The IL-2 conjugate comprises SEQ ID NO: 2, wherein position 64 is AzK_L1_PEG30kD, where AzK_L1_PEG30kD is defined as a structure of Formula (IV) or Formula (V), or a mixture of Formula (IV) and Formula (V), and a 30 kDa, linear mPEG chain. This IL-2 conjugate can also be described as an IL-2 conjugate comprising SEQ ID NO: 1, wherein position 64 is replaced by the structure of Formula (IV) or Formula (V), or a mixture of Formula (IV) and Formula (V), and a 30 kDa, linear mPEG chain. The IL-2 conjugate can also be described as an IL-2 conjugate comprising SEQ ID NO: 1, wherein position 64 is replaced by the structure of Formula (XII) or Formula (XIII), or a mixture of Formula (XII) and Formula (XIII), and a 30 kDa, linear mPEG chain. The compound is prepared as described in Example 1, i.e., using methods wherein a protein was first prepared having SEQ ID NO: 1 in which the proline at position 64 was replaced by N6-((2-azidoethoxy)-carbonyl)-L-lysine AzK. The AzK-containing protein is then allowed to react under click chemistry conditions with DBCO comprising a methoxy, linear PEG group having an average molecular weight of 30 kDa, followed by purification and formulation employing standard procedures.

Cohort B2 participants were patients with platinum-refractory 2L/3L recurrent and/or metastatic HNSCC and who had not received prior treatment with cetuximab. All Cohort B2 participants had been previously treated with a platinum-based regimen and were cetuximab-naïve after failure of no more than 2 regimens for recurrent and/or metastatic (R/M) disease.

Participants were males or females and are aged >18 years. Participants must have had at least one measurable lesion per RECIST v1.1 and a histologically or cytologically confirmed diagnosis of R/M HNSCC that is considered not amenable to further therapy with curative intent (eligible primary tumor locations: oropharynx, oral cavity, hypopharynx, and larynx). Participants with oropharyngeal cancer must have had known human papillomavirus p16 status. Participants must have had adequate cardiovascular, liver, and renal function and laboratory parameters.

Participants were subject to the following exclusion criteria:
ECOG performance status of ≥2
History of allogenic tissue/solid organ transplant
Immune-mediated/related toxicity from prior immunooncology therapy of Grade 4 or leading to discontinuation
Ongoing AEs caused by any prior anti-cancer therapy ≥Grade 2
Baseline oxygen saturation (SpO2)≤92% (without oxygen therapy)
Prior IL2-based anticancer treatment
Cannot temporarily (for at least 36 hours) withhold antihypertensive medications prior to each dose of the IL-2 conjugate
Any medical or clinical condition, laboratory abnormality, or any specific situation as judged by the Investigator that would preclude protocol therapy or would make the subject inappropriate for the study
Prior treatment with cetuximab
Electrolyte (magnesium, calcium, and potassium) levels outside of normal ranges.

Participants of Cohort B2 received the IL-2 conjugate (24 μg/kg dose) once every 3 weeks, and cetuximab by IV infusion according to a dosing regimen as follows. Cetuximab was given on Cycle 1 Day 1 as an initial loading dose of 400 mg/m² infused over 120 minutes (maximum infusion rate 10 mg/min), followed by 250 mg/m² infused over 60 minutes (maximum infusion rate 10 mg/min) for all subsequent doses starting with the Cycle 1 Day 8 administration, until progressive disease (PD). Cetuximab was given on days 1, 8, and 15 of each 21 day cycle. The infusion time of the IL-2 conjugate was about 30 minutes. For the first 4 cycles of treatment, prior to administering the IL-2 conjugate, all participants received IL-2 conjugate premedication to prevent or reduce the acute effect of infusion-associated reactions (IAR) or flu-like symptoms, 30 to 60 minutes prior to infusion of the IL-2 conjugate. The IL-2 conjugate premedication was as follows: acetaminophen (about 650-1000 mg, oral), diphenhydramine (about 25-50 mg, intravenous), and/or ondansetron (about 8 mg or 0.15 mg/kg, intravenous). After the first 4 cycles, administration of the IL-2 conjugate premedication was optional based on the supervising physician's assessment. Prior to administration of the first dose of cetuximab, all participants were pre-medicated with diphenhydramine (about 25 to 50 mg, intravenous). Premedication for subsequent doses of cetuximab was optional based on the supervising physician's assessment. When the IL-2 conjugate and cetuximab were given on the same day, participants who received diphenhydramine as cetuximab premedication may have skipped the diphenhydramine as the IL-2 conjugate premedication. The dosing sequence was as follows: (i) premedication for cetuximab (30-60 min. prior to the start of cetuximab infusion); (ii) cetuximab; (iii) premedication for the IL-2 conjugate (administered 30-60 min. prior to the start of the IL-2 conjugate infusion); and (iv) IL-2 conjugate. Treatment was repeated until PD.

The progression of disease was monitored in patients according to various criteria. The objective response rate (ORR) was evaluated in patients following administration of the IL-2 conjugate and cetuximab combination treatment per RECIST 1.1. The incidence of treatment emergent adverse events (TEAEs), dose-limiting toxicities (DLTs), serious adverse events (SAEs), and laboratory abnormalities were evaluated following administration of the IL-2 conjugate and cetuximab combination treatment according to the National Cancer Institute Common Terminology Criteria for Adverse Events (NCI CTCAE) v 5.0 and the American Society for Transplantation and Cellular Therapy (ASTCT) consensus gradings. The time to complete response (CR) or partial response (PR) per RECIST 1.1 was evaluated for patients following administration of the IL-2 conjugate and cetuximab combination treatment.

The following parameters were also be evaluated in patients following administration of the IL-2 conjugate and cetuximab combination treatment: (1) duration of response (DoR), defined as the time from the first documented evidence of CR or PR until progressive disease (PD) determined per RECIST 1.1 or death from any cause, whichever occurs first; (2) clinical benefit rate (CBR), including confirmed CR or PR at any time or stable disease (SD) of at least 6 months per RECIST 1.1; and (3) progression free survival (PFS), defined as the time from the date of first administration of IL-2 conjugate and cetuximab combination treatment to the date of the first documented tumor progression, as per RECIST 1.1 or death due to any cause, whichever occurs first. Pharmacokinetic parameters, such as concentrations of the IL-2 conjugate and cetuximab, and incidence of anti-drug antibodies (ADAs) against the IL-2 conjugate, were also evaluated in patients at various time points throughout the study. The following additional indicators of anti-tumor activity were also evaluated in patients following administration of the IL-2 conjugate and cetuximab combination treatment: (1) objective response rate by immune Response Evaluation Criteria in Solid Tumors for immune-based therapies (iRECIST); (2) disease control rate (DCR), defined as the proportion of participants who have achieved CR, PR, or SD, per RECIST 1.1; (3) complete response rate (CRR), defined as the proportion of participants who have a confirmed CR, determined per RECIST 1.1; and (4) OS, defined as the time from the first dose of the IL-2 conjugate and cetuximab combination treatment to the date of death due to any cause. The following additional effects on the immune system can also be evaluated in patients following administration of the IL-2 conjugate and cetuximab combination treatment: (1) immune cells expansion and kinetics in blood (CD8+ T-cells and NK cells proliferation (Ki67) and expansion in blood); (2) modulation of immune response in the tumor microenvironment (TME) (programmed death-ligand 1 (PD-L1), CD8+/Ki67, CD4+, FoxP3, and T/NK/Treg expression on baseline and on-treatment tumor tissue samples); (3) kinetics of cytokine production (cytokine panel in blood); and (4) predictive markers of responses (PD-L1, mismatch repair status, tumor mutation burden (TMB), immune gene signature, circulating tumor DNA (ctDNA) on baseline samples).

Results. Seven individuals having R/M HNSCC and having received 1-2 lines of prior treatment (Cohort B2) received the IL-2 conjugate at a dose of 24 μg/kg Q3W in combination with cetuximab (400 mg/m² on day 1, followed by 250 mg/m² QW). Two of the individuals had at least one evaluable post-baseline tumor assessment scan (i.e., were evaluated for efficacy). One subject had a confirmed partial response (i.e., an apparent decrease in the size of target lesions). One subject had a Best Overall Response (BOR) of stable disease. One subject was non-evaluable and discontinued treatment. Evaluations are not yet available for the other subjects

Treatment-emergent adverse events (TEAEs) are summarized in Table 1.

TABLE 1
Treatment Emergent Adverse Events: n = 7.

| System Organ Class | All Grades | Grade ≥3 |
|---|---|---|
| Infections and Infestations | 0/7 (0%) | 0/7 (0%) |
| Blood and lymphatic system disorders | 1/7 (14.3%) | 0/7 (0%) |
| Immune system disorders | 0/7 (0%) | 0/7 (0%) |
| Metabolism and nutrition disorders | 2/7 (28.6%) | 0/7 (0%) |
| Psychiatric disorders | 1/7 (14.3%) | 0/7 (0%) |
| Nervous system disorders | 2/7 (28.6%) | 0/7 (0%) |
| Cardiac disorders | 3/7 (42.9%) | 2/7 (28.6%) |
| Vascular disorders | 1/7 (14.3%) | 0/7 (0%) |
| Respiratory, Thoracic, | 0/7 (0%) | 0/7 (0%) |
| and Mediastinal disorders | | |
| Gastrointestinal disorders | 7/7 (100%) | 1/7 (14.3%) |
| Skin and subcutaneous tissue disorders | 3/7 (42.9%) | 0/7 (0%) |
| Musculoskeletal and | 2/7 (28.6%) | 0/7 (0%) |
| connective tissue disorders | | |
| Renal and Urinary | 0/7 (0%) | 0/7 (0%) |
| General Disorders and | 4/7 (57.1%) | 0/7 (0%) |
| Administration site conditions | | |
| Investigations | 2/7 (28.6%) | 1/7 (14.3%) |
| Injury, Poisoning, and | 4/7 (57.1%) | 0/7 (0%) |
| Procedural Complications | | |

In some embodiments, an individual shows a decrease in the size of target lesion(s) after one cycle of treatment. In some embodiments, an individual shows a decrease in the size of target lesion(s) after the first tumor assessment. In some embodiments, an individual shows a response (i.e., a decrease in the size of target lesions) after the second, third, or fourth tumor assessment. In some embodiments, the individual shows a response (i.e., a decrease in the size of target lesions) after 1, 2, 3, 4, 5, 6, 7, 8, 9, or 10 cycles of treatment. In some embodiments, the individual shows a response (i.e., a decrease in the size of target lesions) after 1, 2, 3, 4, 5, 6, 7, 8, 9, 10, 12, 14, 16, 18, or 20 weeks following the first treatment.

### Example 3. In Vitro Study of IL-2 Conjugate and Cetuximab (PBMC ADCC Assay)

A study was performed to investigate the effects of antibody dependent cellular cytotoxicity (ADCC) by the IL-2 conjugate of Example 2 in combination with cetuximab using a co-culture of human PBMCs with calcein-labeled cancer cell lines (CAL27 and A431).

### CAL27 Cells.

### Reagents.

Bioassay buffer: 1% ultra low IgG FBS added to phenol-red-free RPMI. Complete assay buffer: 450 μL probenecid added to 45 mL bioassay buffer with final probenecid concentration of 77 μg/mL. Calcein-acetoxymethyl ester (Calcein-AM): 50 μg in 25 μL DMSO. Calcein-AM staining buffer: 10 μL Calcein-AM added to 4 mL complete assay buffer (final Calcein-AM concentration of 5 μg/mL). Triton-X-100 lysis buffer: 20 μL Triton-X-100 added to 4 mL complete assay buffer (final concentration of 0.5%).

### Procedure.

On Day 1, a 6-point, 1 in 5 dilution series (in PBS) of the IL-2 conjugate was prepared. The IL-2 conjugate concentrations were 2, 0.4, 0.08, 0.016, 0.0032, and 0 μg/mL. PBMCs were collected by centrifugation at 200×g for 5 minutes and resuspended in phenol red-free RPMI+10% ultra-low IgG at 20 million cells/mL. Appropriate volumes of these PBMCs were transferred to 6 sections of a multi-well reservoir to which a range of the IL-2 conjugate dilutions was added. PBMCs were mixed well with the IL-2 conjugate by pipetting up and down and 50 mL were transferred into round-bottomed 96 well plates using a multi-channel pipette (final PBMC number per well was 1 million). Six empty wells were reserved for controls to be added the following day. The plates were incubated overnight in a humidified incubator at 37° C. in the presence of 5% carbon dioxide.

On Day 2, CAL27 cells (EGFR-expressing oral epithelial squamous cell carcinoma cell line) were harvested using TrypLE express dissociation buffer and collected by centrifugation at 200×g for 5 minutes. Cells were counted and 5 million cells were resuspended in 4 mL calcein-AM staining buffer and incubated for 30 minutes at 37° C. in the presence of 5% carbon dioxide. Cells were then collected and washed twice in complete assay buffer by centrifugation at 200×g for 5 minutes. Cells were counted and resuspended at 0.4 million cells/mL for a final target cell number of 20,000/well.

Cetuximab antibody (Eli Lilly & Co.) was diluted to a working concentration of 3× (3, 0.3, 0.03, 0.003 μg/mL) for final assay concentrations of 1, 0.1, 0.001, 0.0001 μg/mL. The isotype control (hIgG1, Biolegend) was diluted to 3 μg/mL for a final concentration of 1 μg/mL in complete assay buffer. Equal volumes of stained CAL27 cells at 0.4 million cells/mL were mixed with antibody dilutions or isotype control and incubated for 30 minutes at 4° C. to allow antibody to bind. Following incubation, 100 μL of antibody-CAL27 cell mixture were added to the 96 well plates containing 50 μL of the IL-2 conjugate treated PBMCs from Day 1.

Control wells without PBMCs but with 50 μL calcein-AM stained CAL27 cells treated with complete assay buffer (background signal) or stained CAL27 with 50 μL Triton-X-100 treatment (for maximum signal following cell lysis), both made up to 150 μL final volume with complete assay buffer were prepared in triplicate. The plates were centrifuged for 1 minute at 200×g, and then incubated for 60 minutes at 37° C. in the presence of 5% carbon dioxide. After incubation, the plates were again briefly centrifuged before transferring 90 μL of supernatant into fresh black, clear-bottomed plates, and the fluorescence signal was read on an Envision 2104 plate reader (excitation: 492 nm; emission: 515 nm).

The cytotoxicity was calculated using the following formula:

$$Cytotoxicity ( \% ) = ( A - B ) / ( C - B ) \times 100$$

where A is the fluorescence value for treated cells; B is the background from target cells alone; and C is the maximum release valued obtained from Triton-X-100 treatment.

The data represent the % cytotoxicity of the IL-2 conjugate treated human PBMCs on target cancer cells in the presence of cetuximab. The mean percentage from the technical replicates was converted to a proportion. The analysis was conducted using a two-way generalized linear mixed model (GLMM), with factors for the IL-2 conjugate, cetuximab and their interaction, with random donor effects, treating proportion as a pseudo-binomial variable. It was followed by a post-hoc test (with Dunnett-Hsu adjustment) to compare the IL-2 conjugate treated groups to the control group. Statistical analyses were performed using SAS (1) version 9.4 software. A probability less than 5% (p<0.05) was considered as significant.

### Results.

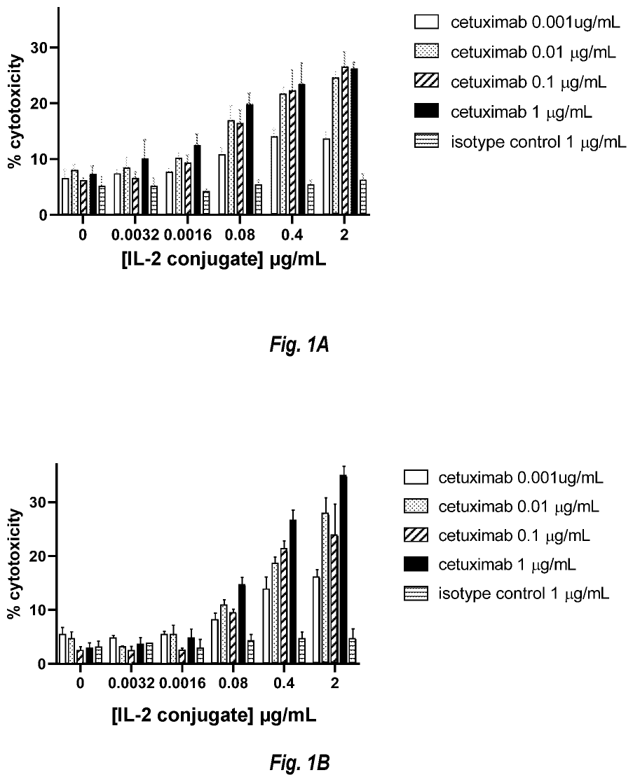

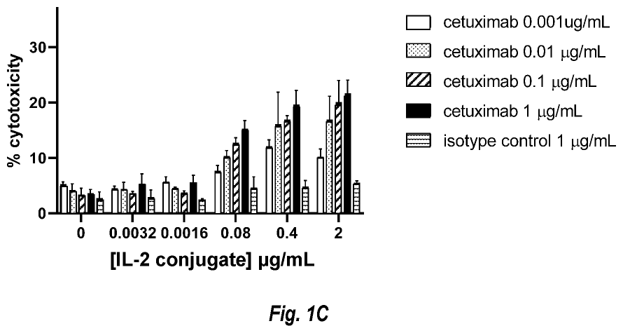

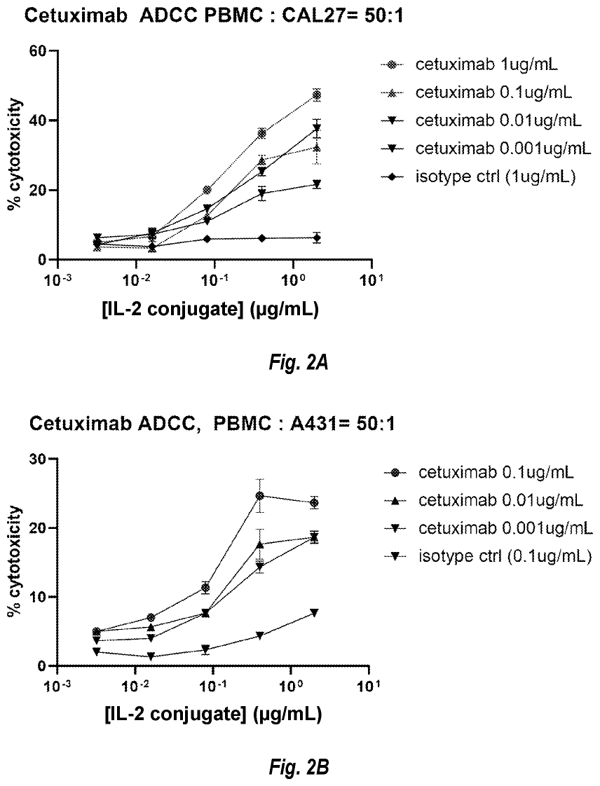

At cetuximab dose levels of 1, 0.1, and 0.01 mg/mL, the IL-2 conjugate enhanced ADCC function of cetuximab against EGFR expressing CAL27 cells (p<0.05) at concentrations of 0.08, 0.4 and 2 mg/mL (FIGS. 1A-C). At a cetuximab dose level of 0.001 mg/mL, the IL-2 conjugate enhanced ADCC function of cetuximab against EGFR expressing CAL27 cells (p<0.05) at concentrations of 0.4 and 2 mg/mL. FIG. 2A further shows the enhanced ADCC function of cetuximab against EGFR expressing CAL27 cells (PBMC to CAL27 ratio 50:1).

The tests of fixed effects from the GLMM model indicate that the factors IL-2 conjugate, cetuximab and their interaction have a significant effect on the cytotoxicity, i.e., the differences between IL-2 conjugate groups vary significantly for the different cetuximab concentrations. The pairwise comparisons indicated a significant difference between the IL-2 conjugate 2 mg/mL group versus the control group (p=0.0001) and between the IL-2 conjugate 0.4 mg/mL group versus the control group (p=0.0001) at a cetuximab concentration of 0.001 mg/mL. The pairwise comparisons also indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p=0.0003) at a cetuximab concentration of 0.01 mg/mL. In addition, the pairwise comparisons indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p<0.0001) at a cetuximab concentration of 0.1 mg/mL. Lastly, the pairwise comparisons indicated a significant difference between the IL-2 conjugate 2 mg/mL group versus the control group (p<0.0001), between the IL-2 conjugate 0.4 mg/mL group versus the control group (p<0.0001), and between the IL-2 conjugate 0.08 mg/mL group versus the control group (p<0.0001) at a cetuximab concentration of 1 mg/mL.

The data demonstrate that the IL-2 conjugate enhanced ADCC function of cetuximab against EGFR expressing CAL27 cancer cells. No significant differences were observed using the IL-2 conjugate in combination with the isotype control.

### A431 Cells.

Studies were performed using EGFR expressing A431 cells (epidermoid carcinoma) following the procedure outlined above for CAL27 cells. FIG. 2B shows the enhanced ADCC function of cetuximab against EGFR expressing A431 cells (PBMC to A431 ratio 50:1). The data demonstrate that the IL-2 conjugate enhanced ADCC function of cetuximab against EGFR expressing A431 cancer cells.

### Example 4. ADCC Assay Using an Engineered Cell Line NK-92.CD16 V as Effector Cells

The effect of the IL-2 conjugate of Example 1 on ADCC function of cetuximab was examined using a calcein-acetyoxymethyl (Calcein-AM; Invitrogen) release assay.

### Materials.

NK-92.CD16 V (high affinity variant) (Conkwest Inc., San Diego, CA) was used as the effector cell line. The following cell lines were used as target cells: CAL27, A431, DLD-1, and FaDu.

The following reagents were used: cetuximab antibody (Eli Lilly & Co.); human isotype IgG1 antibody (Biolegend); calcein-acetyoxymethyl (Calcein-AM; Invitrogen C3100MP), and probenecid (Invitrogen; P36400). The bioassay medium was phenol red-free RPMI with 1% ultra low IgG fetal bovine serum, supplemented with 1% probenecid for complete assay medium. MyeloCult H5100 (Stemcell Cat #05150) supplemented with IL-2 (100 U/mL) and hydrocortisone (Sigma H6909; 10 mL at 50 μM) was used for the NK-92.CD16 V cell culture.

### Procedure.

IL-2 supplement was withdrawn from the NK-92.CD16 V cell culture, which was then incubated overnight prior to starting the assay. The next day, cells were plated in 96-well round-bottom plates (60,000 cells were plated for a 3:1 ratio of effector to target cells) in the presence of IL-2_P65_[AzK_L1_PEG30kD]-1 at varying concentrations (0.1 μg/mL, 0.01 μg/mL, 0.001 μg/mL, and 0 μg/mL) in phenol red-free RPMI 1640 media supplemented with 1% low IgG FBS for 18 hours at 37° C. in a humidified incubator with 5% CO₂. These cells are used as the effector cells. The following day, human EGFR positive cancer cell lines (A431, DLD-1, FaDu, or CAL27) were labeled with calcein-AM for 30 min (50 μg diluted in 25 μL DMSO to prepare a stock solution, then 10 μL of calcein stock solution was added to 4 mL RPMI 1640 containing 1% low IgG FBS and 1% probenecid for the staining of 5×10⁶ cells) and then washed. Cells were divided into several labeled tubes for incubation with varying concentrations of cetuximab or isotype control. Cetuximab and isotype human IgG1 antibody were added at 3× concentrations (for final assay concentrations from 10 μg/mL to 1 μg/mL), and the labeled target cells and antibody were mixed and allowed to sit for 30 min to allow opsonization. After this incubation, target cells (20,000) and antibody were added on top of NK-92.CD16 V cells in 100 μL. The plate was centrifuged briefly for 1 minute at 1100 rpm before incubating at 37° C. and 5% CO₂ for 1 hour.

Following incubation, the plates were again briefly centrifuged as before, and 90 μL of supernatant was transferred from each well to black plates with clear bottom without disturbing the cells. The fluorescence signal was read using Envision 2104 (excitation: 492 nm; emission: 515 nm).

For maximal release, the cells were lysed with 2% Triton X-100. The fluorescence value of the culture medium background was subtracted from that of the experimental release (A), the target cell spontaneous release (B), and the target cell maximal release (C).

The cytotoxicity and ADCC percentages for each plate (in duplicate) were calculated using the following formulas:

Cytotoxicity (%)=(A−B)/(C−B)×100

ADCC (%)=Cytotoxicity (%, with antibody) −Cytotoxicity (%, without antibody)

For each experiment, measurements were conducted in triplicate using three replicate wells. Each experiment is repeated at least 3 times. The half-maximal effective concentration (EC50) values are calculated by fitting the data points to a 4-parameter equation using GraphPad Prism 5 (GraphPad Software, Inc., San Diego, CA).

### Results.

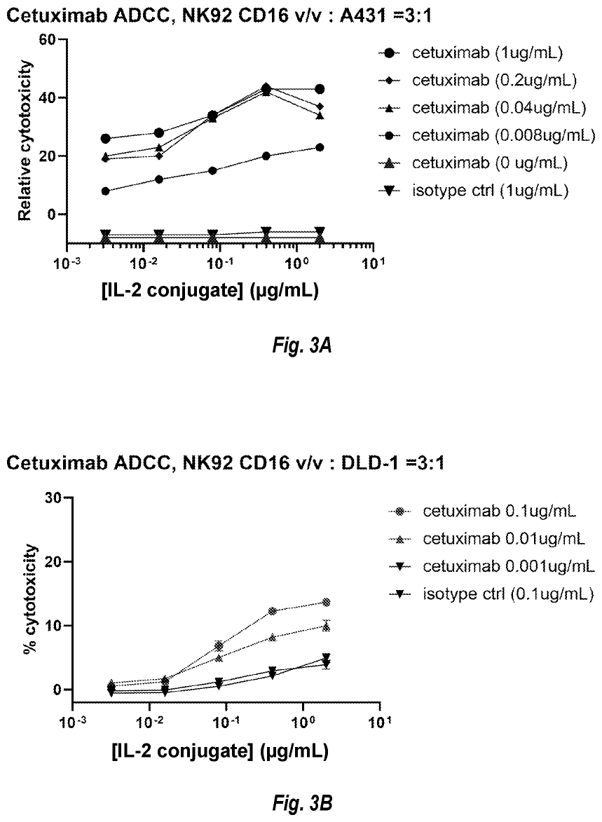

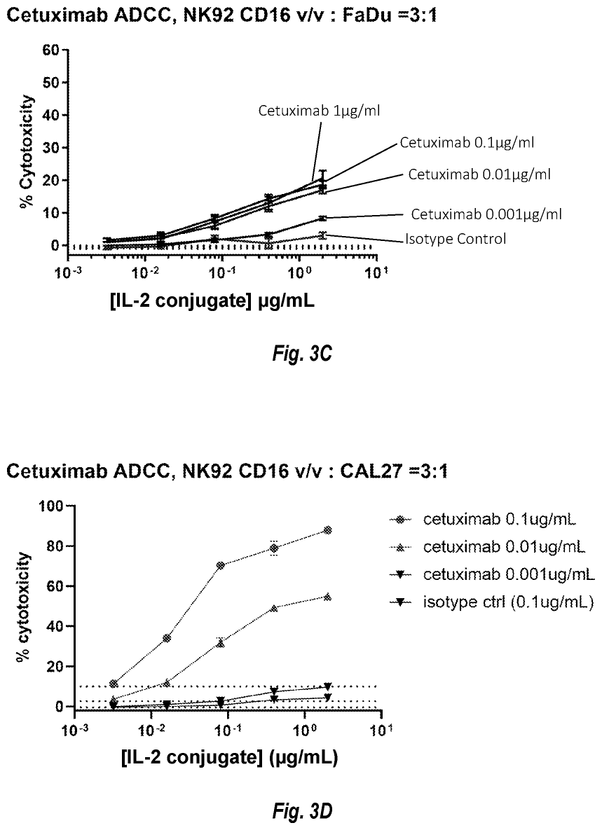

Cytotoxicity data using the NK92 cell line ADCC assay is shown in FIGS. 3A-D for EGFR expressing A431 (epidermoid carcinoma) (NK92 to A431 ratio 3:1), DLD-1 (adenocarcinoma, colorectal) (NK92 to DLD-1 ratio 3:1), FaDu (epithelial squamous cell carcinoma) (NK92 to FaDu ratio 3:1), and CAL27 (epithelial squamous cell carcinoma) (NK92 to CAL27 ratio 3:1) cells, respectively. The data demonstrate that the IL-2 conjugate enhanced ADCC function of cetuximab against EGFR expressing cancer cells.

### Example 5. Clinical Study of Combination Therapy Using an IL-2 Conjugate and Cetuximab

Overview. Monotherapy using the IL-2 conjugate of Example 2 has been demonstrated to promote a peripheral increase in the number of NK cells, which are important effector cells mediating antibody-dependent cellular cytotoxicity (ADCC) for IgG1 antibodies such as cetuximab. See, e.g., WO 2022/076859 A1 to Caffaro et al., published Apr. 14, 2022, which is incorporated herein by reference for all purposes.

A Phase 1/2, open-label, multi-center study assessing the clinical benefit of the IL-2 conjugate described in Example 2 in combination with cetuximab for the treatment of participants with advanced or metastatic solid tumors was undertaken.

A total of 23 participants received the IL-2 conjugate (16, 24, or 32 μg/kg dose) by IV infusion once every 3 weeks. Here and throughout discussion of this and all other Examples, drug mass per kg subject (e.g., 16 μg/kg) refers to IL-2 mass exclusive of PEG and linker mass. Cetuximab was given on Cycle 1 Day 1 as an initial loading dose of 400 mg/m² infused over 120 minutes (maximum infusion rate 10 mg/min), followed by 250 mg/m² infused over 60 minutes (maximum infusion rate 10 mg/min) for all subsequent doses starting with the Cycle 1 Day 8 administration. Cetuximab was given on days 1, 8, and 15 of each 21 day cycle. The infusion time of the IL-2 conjugate was about 30 minutes each. For each cycle of treatment, prior to administering the IL-2 conjugate, at least one participant received IL-2 conjugate premedication to prevent or reduce the acute effect of infusion-associated reactions (IAR) or flu-like symptoms, 30 to 60 minutes prior to infusion of the IL-2 conjugate. The IL-2 conjugate premedication was as follows: anti-pyretic, orally, and anti-histamine (H1 blocker). Antiemetics were provided at the discretion of the supervising physician. Prior to administration of the first dose of cetuximab, at least one participant were pre-medicated with diphenhydramine (about 25 to 50 mg, intravenous). Premedication for subsequent doses of cetuximab was optional based on the supervising physician's assessment. When the IL-2 conjugate and cetuximab were given on the same day, participants who received diphenhydramine as cetuximab premedication may have skipped the diphenhydramine as the IL-2 conjugate premedication. The dosing sequence was as follows: (i) premedication for cetuximab (30-60 min. prior to the start of cetuximab infusion); (ii) cetuximab; (iii) premedication for the IL-2 conjugate (administered 30-60 min. prior to the start of the IL-2 conjugate infusion); and (iv) IL-2 conjugate. Treatment was repeated for up to a total of 35 cycles or for a duration up to 735 days.

Subjects ranged in age from 43 to 75 with a mean age 60.7 and a median age of 65.0. All subjects had metastatic disease. 14 subjects were male and 8 were female. 1 subject was Hispanic or Latino, 20 were not Hispanic or Latino and 2 were not reported. 11 subjects were White, 1 was Black or African American, 7 were Asian, 4 were Other and 1 was not reported. 5 subjects had an ECOG score of 0 and 18 had an ECOG score of 1. Prior lines of systemic therapies were as follows: 1 subject had 1 line; 4 subjects had 2 lines; 4 subjects had 3 lines; 7 subjects had 4 lines; and 6 subjects had 5+ lines. Primary tumor types included 11 colorectal cancer (CRC), 1 non-small cell lung carcinoma (NSCLC), 1 HNSCC, and 12 Other.

The following biomarkers serve as surrogate predictors of safety and/or efficacy:
Eosinophilia (elevated peripheral eosinophil count): Cell surrogate marker for IL-2-induced proliferation of cells (eosinophils) linked to vascular leak syndrome (VLS);
Interleukin 5 (IL-5): Cytokine surrogate marker for IL-2 induced activation of type 2 innate lymphoid cells and release of this chemoattractant that leads to eosinophilia and potentially VLS;
Interleukin 6 (IL-6): Cytokine surrogate marker for IL-2 induced cytokine release syndrome (CRS); and
Interferon γ (IFN-γ): Cytokine surrogate marker for IL-2 induced activation of CD8+ cytotoxic T lymphocytes.

The following biomarkers serve as surrogate predictors of anti-tumor immune activity:
Peripheral CD8+ Effector Cells: Marker for IL-2-induced proliferation of these target cells in the periphery that upon infiltration become a surrogate marker of inducing a potentially latent therapeutic response;
Peripheral CD8+ Memory Cells: Marker for IL-2-induced proliferation of these target cells in the periphery that upon infiltration become a surrogate marker of inducing a potentially durable latent therapeutic and maintenance of the memory population;
Peripheral NK Cells: Marker for IL-2-induced proliferation of these target cells in the periphery that upon infiltration become a surrogate marker of inducing a potentially rapid therapeutic response; and
Peripheral CD4+ Regulatory Cells: Marker for IL-2-induced proliferation of these target cells in the periphery that upon infiltration become a surrogate marker of inducing an immunosuppressive TME and offsetting of an effector-based therapeutic effect.
   First Cohort Using 16 μg/Kg Dose of IL-2 Conjugate

Results. The 5 subjects included two human males and 3 females with a median age of 69 years (ranging from 65-72 years). All subjects had an Eastern Cooperative Oncology Group (ECOG) performance status of 0 or 1, and had received 1 to 4 prior lines of systemic therapies. The cancers were anal cancer (1 subject), colon adenocarcinoma (1 subject), adrenocortical cancer (1 subject), squamous cell carcinoma of the lung (1 subject), and small intestinal cancer (1 subject). All five subjects had metastatic disease.

The subjects received the IL-2 conjugate (16 μg/kg) and cetuximab combination treatment for 2-7 cycles (2-7 doses of the IL-2 conjugate). Two subjects, one with anal cancer and one with metastatic adrenocortical carcinoma, showed progressive disease (PD) following 2 cycles of combination treatment, leading to discontinuation of the IL-2 conjugate and cetuximab combination treatment. One subject with colon cancer showed disease progression after 5 cycles. Two subjects are ongoing: one at 3 cycles with squamous cell carcinoma of the lung and one at 7 cycles with small intestinal carcinoma.

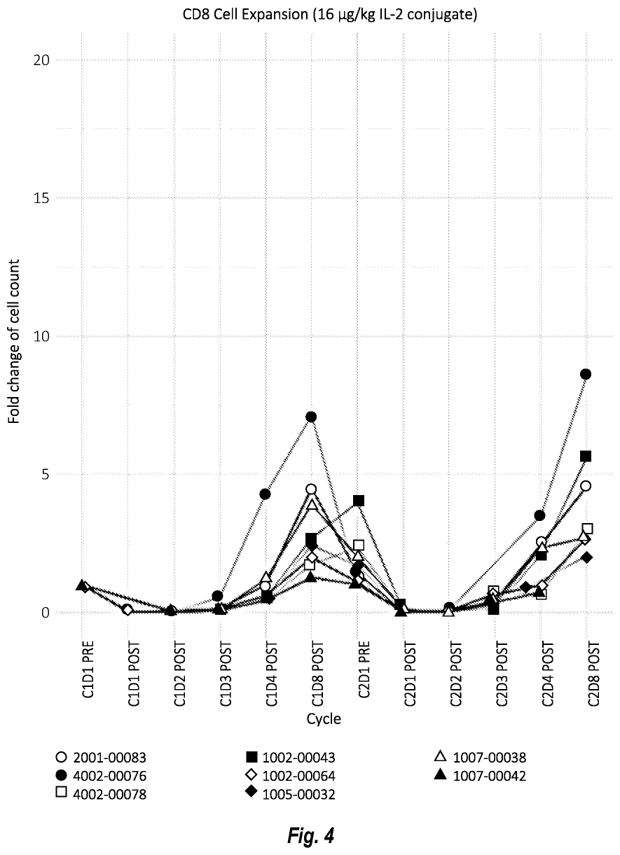

Peripheral CD8+T_{eff} cell counts were measured (FIG. 4). Prolonged CD8+ expansion over baseline (e.g., greater than or equal to 2-fold change) was observed at 3 weeks after the previous dose in some subjects.

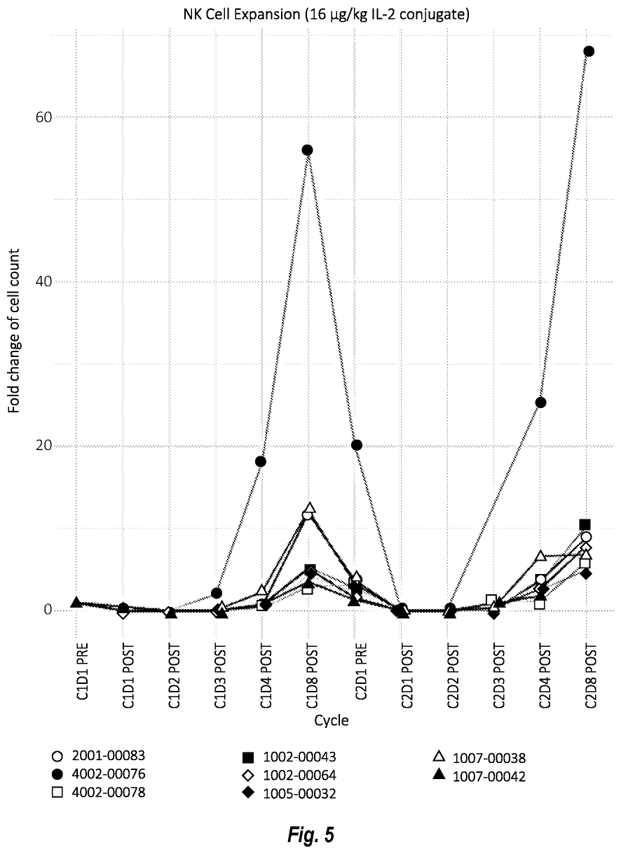

Peripheral NK cell counts are shown in FIG. 5. An increase in NK cell count was observed in each subject. Subjects generally showed elevated NK cell counts over baseline at 8 days and 3 weeks after the previous dose.

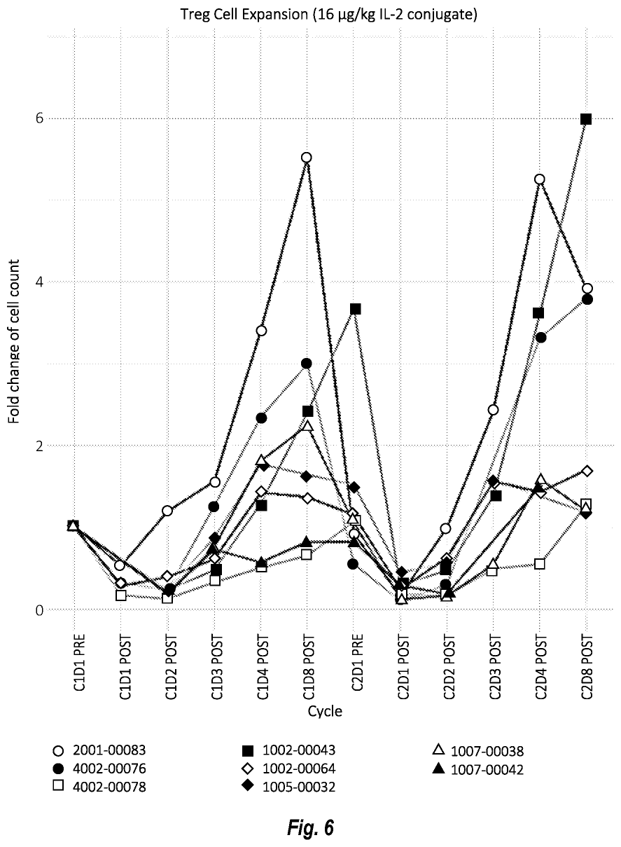

Peripheral CD4+T_{reg} counts are shown in FIG. 6.

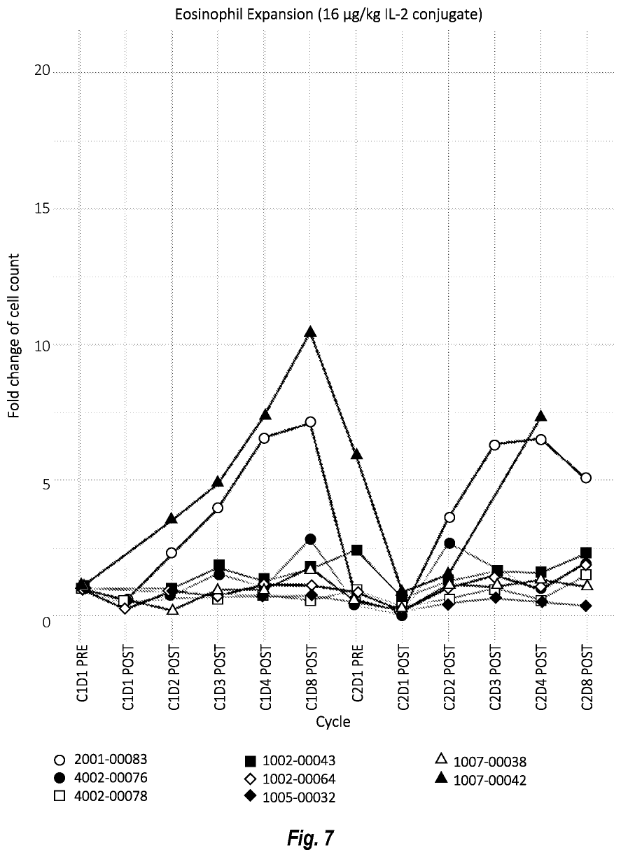

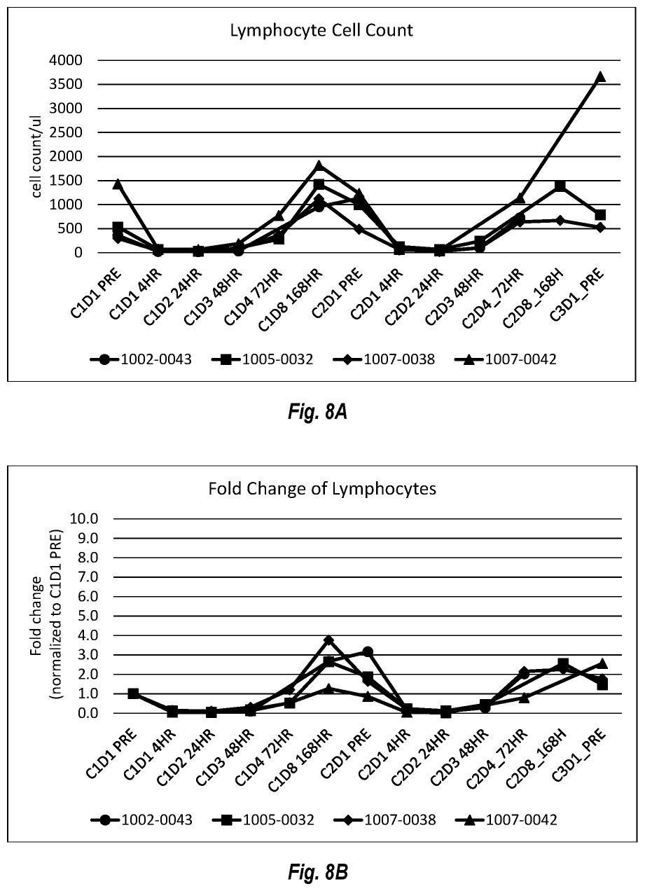

Eosinophil counts were measured (FIG. 7). The measured values did not exceed a four-fold increase and were consistently below the range of 2328-15958 eosinophils/μL in patients with IL-2 induced eosinophilia as reported in Pisani et al., Blood 1991 Sep. 15; 78(6):1538-44. Lymphocyte counts were also measured (FIGS. 8A-B).

Summary of Results and Discussion. All subjects tested had post-dose peripheral expansion of CD8+T effector (Teff) cells, NK cells, and CD4+T_{reg} cells.

An adverse event (AE) was any untoward medical occurrence in a clinical investigation subject administered a pharmaceutical product, regardless of causal attribution. Dose-limiting toxicities were defined as an AE occurring within Day 1 through Day 29 (inclusive)+1 day of a treatment cycle that was not clearly or incontrovertibly solely related to an extraneous cause and that met at least one of the following criteria:
Grade 3 neutropenia (absolute neutrophil count <1000/mm³>500/mm³) lasting >7 days, or Grade 4 neutropenia of any duration
Grade 3+ febrile neutropenia
Grade 4+ thrombocytopenia (platelet count <25,000/mm³)
Grade 3+ thrombocytopenia (platelet count <50,000-25,000/mm³) lasting >5 days, or associated with clinically significant bleeding or requiring platelet transfusion
Failure to meet recovery criteria of an absolute neutrophil count of at least 1,000 cells/mm³ and a platelet count of at least 75,000 cells/mm³ within 10 days
Any other grade 4+ hematologic toxicity lasting ≥5 days
Grade 3+ ALT or AST in combination with a bilirubin >2 times ULN with no evidence of cholestasis or another cause such as viral infection or other drugs (i.e. Hy's law)
Grade 3 infusion-related reaction that occurs with premedication; Grade 4 infusion-related reaction
Grade 3 Vascular Leak Syndrome defined as hypotension associated with fluid retention and pulmonary edema
Grade 3+ anaphylaxis
Grade 3+ hypotension
Grade 3+AE that does not resolve to grade <2 within 7 days of starting accepted standard of care medical management
Grade 3+ cytokine release syndrome

The following exceptions applied to non-hematologic AEs:
Grade 3 fatigue, nausea, vomiting, or diarrhea that resolves to grade ≤2 with optimal medical management in ≤3 days
Grade 3 fever (as defined by >40° C. for ≤24 hours)
Grade 3 infusion-related reaction that occurs without premedication; subsequent doses should use premedication and if reaction recurs then it will be a DLT
Grade 3 arthralgia or rash that resolves to grade ≤2 within 7 days of starting accepted standard of care medical management (e.g., systemic corticosteroid therapy)

If a subject had grade 1 or 2 ALT or AST elevation at baseline considered secondhand to liver metastases, a grade 3 elevation must also be ≥3 times baseline and last >7 days.

Serious AEs were defined as any AE that results in any of the following outcomes: death; life-threatening AE; inpatient hospitalization or prolongation of an existing hospitalization; a persistent or significant incapacity or substantial disruption of the ability to conduct normal life functions; or a congenital anomaly/birth defect. Important medical events that may not result in death, be life-threatening, or require hospitalization may be considered serious when, based upon appropriate medical judgment, they may jeopardize the subject and may require medical or surgical intervention to prevent one of the outcomes listed above. Examples of such medical events include allergic bronchospasm requiring intensive treatment in an emergency room or at home, blood dyscrasias or convulsions that do not result in inpatient hospitalization, or the development of drug dependency or drug abuse.

There were no meaningful elevations in IL-5. There was no cumulative toxicity. There was no end organ toxicity. There was no QTc prolongation or other cardiac toxicity. Overall, the IL-2 conjugate was considered well-tolerated.

Four of the 5 subjects had at least one treatment-emergent AE (TEAE). Most of the TEAEs were Grade 1-2, one subject had at least one Grade 3, and one subject at least one Grade 4 TEAE. Four subjects had treatment related AEs. These included: one Grade 1 infusion reaction; one Grade 1 nausea; one Grade 1 fatigue; one Grade 2 diarrhea; and one Grade 4 lymphocyte count decrease. Two subjects had 3 unrelated SAEs: one dysphagia and spinal cord compression; and one pleural effusion. The TEAEs did not result in any drug discontinuations, no dose reductions, no DLTs, and no anaphylaxis or CRS. The treatment-related AEs resolved with accepted standard of care. TEAEs are detailed in Table 2, and treatment-related adverse events are summarized in Table 3.

TABLE 2
Treatment Emergent Adverse Events: n = 5.

| System Organ Class | Grade 1 | Grade 2 | Grade 3 | Grade 4 | Grade 5 |
|---|---|---|---|---|---|
| General disorders and | 2/5 (40%) | 0/5 (0%) | 0/5 (0%) | 0/5 (0%) | 0/5 (0%) |
| administration site conditions | | | | | |
| Gastrointestinal disorders | 2/5 (40%) | 0/5 (0%) | 1/5 (20%) | 0/5 (0%) | 0/5 (0%) |
| Investigations | 0/5 (0%) | 0/5 (0%) | 0/5 (0%) | 1/5 (20%) | 0/5 (0%) |
| Infections and infestations | 0/5 (0%) | 1/5 (20%) | 0/5 (0%) | 0/5 (0%) | 0/5 (0%) |
| Injury, Procedural | 2/5 (40%) | 0/5 (0%) | 0/5 (0%) | 0/5 (0%) | 0/5 (0%) |
| Complications | | | | | |
| Nervous system disorders | 0/5 (0%) | 0/5 (0%) | 2/5 (40%) | 0/5 (0%) | 0/5 (0%) |
| Skin and subcutaneous tissue | 3/5 (60%) | 0/5 (0%) | 0/5 (0%) | 0/5 (0%) | 0/5 (0%) |
| disorders | | | | | |
| Blood and lymphatic system | 1/5 (20%) | 0/5 (0%) | 0/5 (0%) | 0/5 (0%) | 0/5 (0%) |
| disorders | | | | | |
| Eye Disorders | 1/5 (20%) | 0/5 (0%) | 0/5 (0%) | 0/5 (0%) | 0/5 (0%) |
| Metabolism and nutrition | 1/5 (20%) | 0/5 (0%) | 0/5 (0%) | 0/5 (0%) | 0/5 (0%) |
| disorders | | | | | |
| Respiratory, thoracic and | 0/5 (0%) | 1/5 (20%) | 0/5 (0%) | 0/5 (0%) | 0/5 (0%) |
| mediastinal disorders | | | | | |
| Endocrine Disorders | 0/5 (0%) | 1/5 (20%) | 0/5 (0%) | 0/5 (0%) | 0/5 (0%) |
| Musculoskeletal and | 1/5 (20%) | 1/5 (20%) | 0/5 (0%) | 0/5 (0%) | 0/5 (0%) |
| connective tissue disorders | | | | | |
| Psychiatric disorders | 1/5 (20%) | 0/5 (0%) | 0/5 (0%) | 0/5 (0%) | 0/5 (0%) |
| Reproductive and Breast | 1/5 (20%) | 0/5 (0%) | 0/5 (0%) | 0/5 (0%) | 0/5 (0%) |
| Disorders | | | | | |
| Renal and Urinary Disorders | 1/5 (20%) | 0/5 (0%) | 0/5 (0%) | 0/5 (0%) | 0/5 (0%) |

TABLE 3
Treatment Related Adverse Events: n = 5.

| System Organ Class | Grade 1 | Grade 2 | Grade 3 | Grade 4 | Grade 5 |
|---|---|---|---|---|---|
| Endocrine Disorders | 0/5 (0%) | 1/5 (20%) | 0/5 (0%) | 0/5 (0%) | 0/5 (0%) |
| General disorders and | 2/5 (40%) | 0/5 (0%) | 0/5 (0%) | 0/5 (0%) | 0/5 (0%) |
| administration site conditions | | | | | |
| Gastrointestinal disorders | 1/5 (20%) | 1/5 (205%) | 0/5 (0%) | 0/5 (0%) | 0/5 (0%) |
| Investigations | 0/5 (0%) | 0/5 (0%) | 0/5 (0%) | 1/5 (20%) | 0/5 (0%) |
| Injury, Procedural | 1/5 (20%) | 0/5 (0%) | 0/5 (0%) | 0/5 (0%) | 0/5 (0%) |
| Complications | | | | | |
| Nervous system disorders | 0/5 (0%) | 0/5 (0%) | 1/5 (20%) | 0/5 (0%) | 0/5 (0%) |

Second Cohort Using 24 μg/Kg Dose of IL-2 Conjugate

Results. Five subjects received the IL-2 conjugate at a 24 μg/kg dosage. All subjects had an Eastern Cooperative Oncology Group (ECOG) performance status of 1. One subject had received 1 prior line of therapy, and a second subject had received 4 prior lines of therapy. The cancers included squamous cell carcinoma (2 subjects, one of which was head and neck squamous cell carcinoma (HNSCC)) and adenocarcinoma (3 subjects, two of which were colon adenocarcinoma). All of the subjects had advanced and/or metastatic disease. The subjects received the IL-2 conjugate (24 μg/kg) and cetuximab combination treatment for 1, 2, or 3 cycles (each cycle having 1 dose of the IL-2 conjugate). One subject showed progressive disease (PD) following the first cycle of combination treatment, and a second subject showed progressive disease (PD) following the second cycle of combination treatment, preventing administration of a further treatment dose of the IL-2 conjugate and cetuximab combination treatment. Progressive disease was not observed in the other three subjects as of 1, 2, and 3 cycles of treatment.

One 75-year-old subject with HNSCC achieved a partial response by 3 cycles of treatment (41% decrease tumor size), which was again confirmed at 5 cycles of treatment (70% decrease tumor size). Progressive decrease was not observed as of 18 cycles of treatment. This subject had previously undergonet radiotherapy, surgery and two lines of systemic therapy including cisplatin (for a duration of about six weeks) as well as radiotherapy and resection. Following recurrence of the HNSCC, the subject had received cemiplimab (for a duration of about three months) with a best response of progressive disease.

All five initial subjects experienced at least one TEAE. Three subjects had a Grade 3 TEAEs (one each of chills, pyrexia, and vomiting). There were related SAEs of nausea, vomiting and tachycardia requiring hospitalization, which resolved with supportive care. One subject experienced elevation of IL-6 to about 1876 μg/mL without symptoms.

At least in the initial subjects, No DLTs were observed and no drug discontinuations resulted from the TEAEs. TEAEs are detailed for the first three subjects enrolled in Table 4, and treatment-related adverse events for the first three subjects enrolled are summarized in Table 5.

TABLE 4
Treatment Emergent Adverse Events: n = 3.

| System Organ Class | Grade 1 | Grade 2 | Grade 3 | Grade 4 | Grade 5 |
|---|---|---|---|---|---|
| Gastrointestinal disorders | 0/3 (0%) | 1/3 (33.3%) | 0/3 (0%) | 0/3 (0%) | 0/3 (0%) |
| General disorders and | 1/3 (33.3%) | 0/3 (0%) | 1/3 (33.3%) | 0/3 (0%) | 0/3 (0%) |
| administration conditions | | | | | |
| Infections and infestations | 0/3 (0%) | 2/3 (66.7%) | 0/3 (0%) | 0/3 (0%) | 0/3 (0%) |
| Metabolism and nutrition | 0/3 (0%) | 2/3 (66.7%) | 0/3 (0%) | 0/3 (0%) | 0/3 (0%) |
| disorders | | | | | |
| Musculoskeletal and | 1/3 (33.3%) | 0/3 (0%) | 0/3 (0%) | 0/3 (0%) | 0/3 (0%) |
| connective tissue disorders | | | | | |
| Nervous system disorders | 0/3 (0%) | 1/3 (33.3%) | 0/3 (0%) | 0/3 (0%) | 0/3 (0%) |
| Renal and urinary disorders | 1/3 (33.3%) | 0/3 (0%) | 0/3 (0%) | 0/3 (0%) | 0/3 (0%) |
| Respiratory and mediastinal | 0/3 (0%) | 1/3 (33.3%) | 0/3 (0%) | 0/3 (0%) | 0/3 (0%) |
| disorders | | | | | |
| Skin and subcutaneous | 1/3 (33.3%) | 1/3 (33.3%) | 0/3 (0%) | 0/3 (0%) | 0/3 (0%) |
| tissue disorders | | | | | |

TABLE 5
Treatment Related Adverse Events: n = 3.

| System Organ Class | Grade 1 | Grade 2 | Grade 3 | Grade 4 | Grade 5 |
|---|---|---|---|---|---|
| Gastrointestinal disorders | 0/3 (0%) | 1/3 (33.3%) | 0/3 (0%) | 0/3 (0%) | 0/3 (0%) |
| General disorders and | 1/3 (33.3%) | 0/3 (0%) | 1/3 (33.3%) | 0/3 (0%) | 0/3 (0%) |
| administration conditions | | | | | |
| Infections and infestations | 0/3 (0%) | 2/3 (66.7%) | 0/3 (0%) | 0/3 (0%) | 0/3 (0%) |
| Metabolism and nutrition | 0/3 (0%) | 2/3 (66.7%) | 0/3 (0%) | 0/3 (0%) | 0/3 (0%) |
| disorders | | | | | |
| Nervous system disorders | 0/3 (0%) | 1/3 (33.3%) | 0/3 (0%) | 0/3 (0%) | 0/3 (0%) |
| Respiratory and mediastinal | 0/3 (0%) | 1/3 (33.3%) | 0/3 (0%) | 0/3 (0%) | 0/3 (0%) |
| disorders | | | | | |
| Skin and subcutaneous | 0/3 (0%) | 1/3 (33.3%) | 0/3 (0%) | 0/3 (0%) | 0/3 (0%) |
| tissue disorders | | | | | |

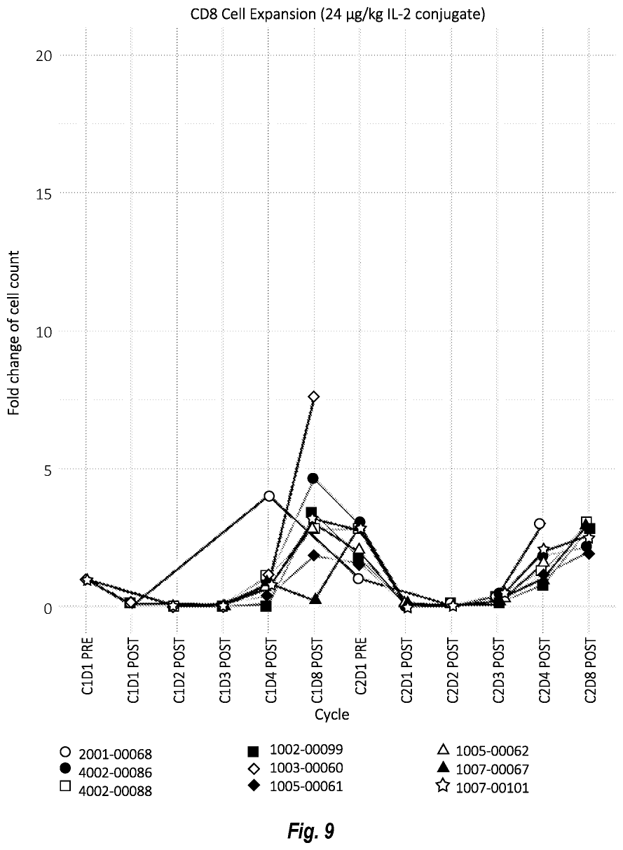

Peripheral CD8+ Teff cell counts were measured (FIG. 9). CD8+ expansion over baseline (e.g., about 2-fold change) was observed in some subjects.

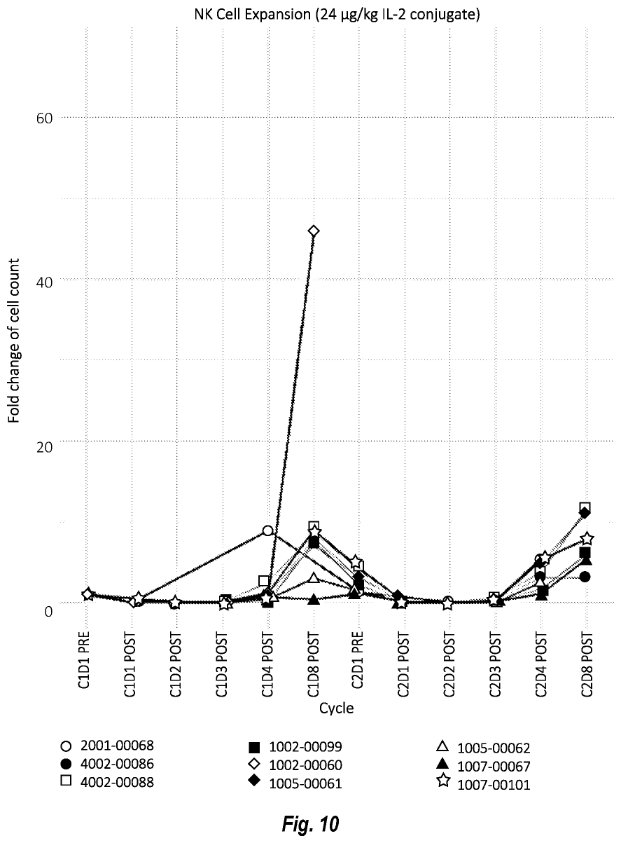

Peripheral NK cell counts are shown in FIG. 10. An increase in NK cell count was observed in each subject. Subjects generally showed elevated NK cell counts over baseline at one or more time points after dosing.

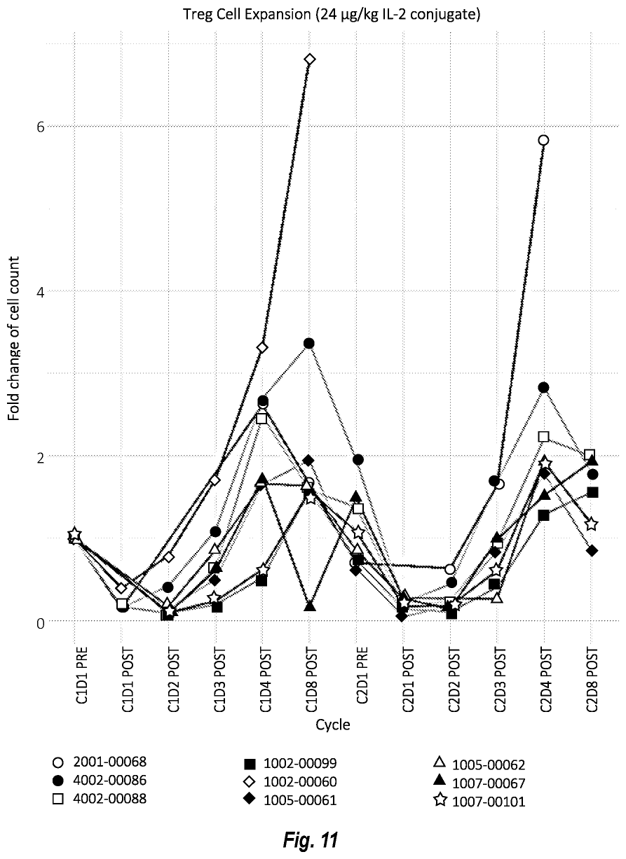

Peripheral CD4+T_{reg} counts are shown in FIG. 11.

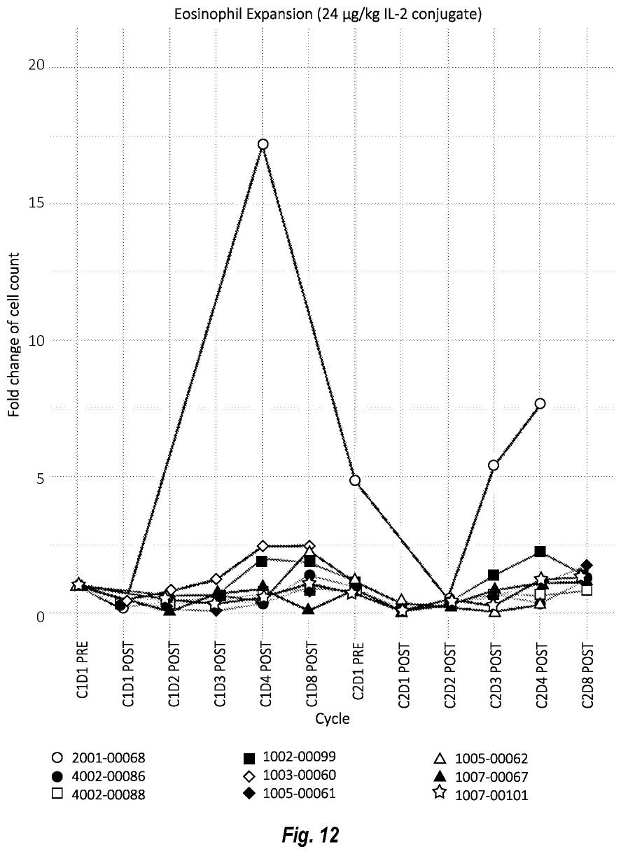

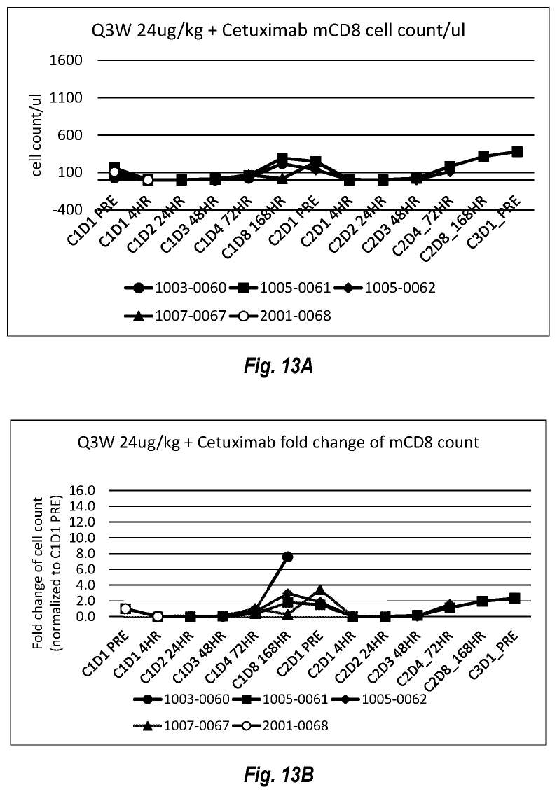

Eosinophil counts were measured (FIG. 12). The measured values did not exceed a three-fold increase and were consistently below the range of 2328-15958 eosinophils/μL in patients with IL-2 induced eosinophilia as reported in Pisani et al., Blood 1991 Sep. 15; 78(6):1538-44. mCD8 Lymphocyte counts were also measured (FIGS. 13A-B).

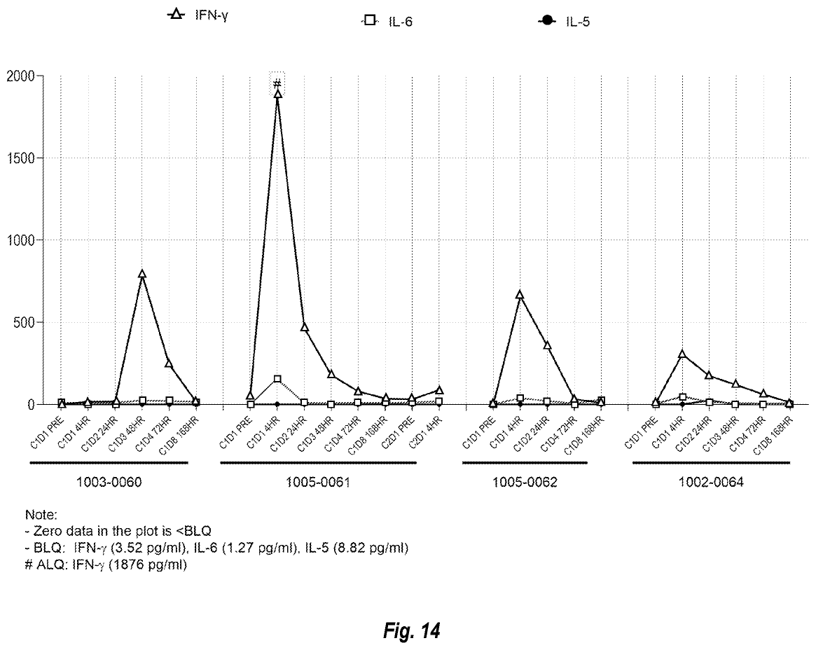

IFN-γ, IL-6, and IL-5 serum levels for four of the subjects are shown in FIG. 14. As mentioned above, the subject who experienced an IL-6 increase to about 1876 μg/mL did not experience an IL-6 associated AE.

Third Cohort Using 32 μg/Kg Dose of IL-2 Conjugate

Six subjects having advanced and/or metastatic cancers, including colorectal cancer (e.g., colorectal adenocarcinoma), colon cancer, ampullary cancer, esophageal squamous cell carcinoma, and pancreatic adenocarcinoma, received the IL-2 conjugate at a 32 μg/kg dosage. The subjects received the IL-2 conjugate (32 μg/kg) and cetuximab combination treatment for 2-5 cycles (2-5 doses of the IL-2 conjugate). One subject stopped receiving treatment for PD after Cycle 2. Another subject received pre-medication with granisetron and diphenhydramine and stopped receiving treatment for PD. For another subject, scans showed disease progression, but the subject continued treatment post disease progression to at least Cycle 3; the subject was subsequently documented to have disease progression. Another subject (colorectal adenocarcinoma) is ongoing at Cycle 5. One subject achieved a best response of stable disease. One of these subjects (pancreatic adenocarcinoma) continued therapy until PD at Cycle 4, Day 15.

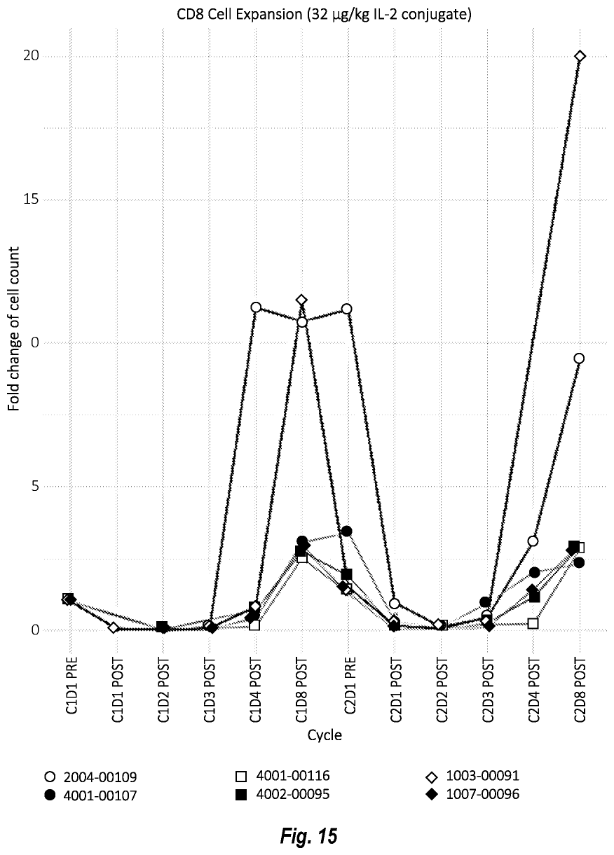

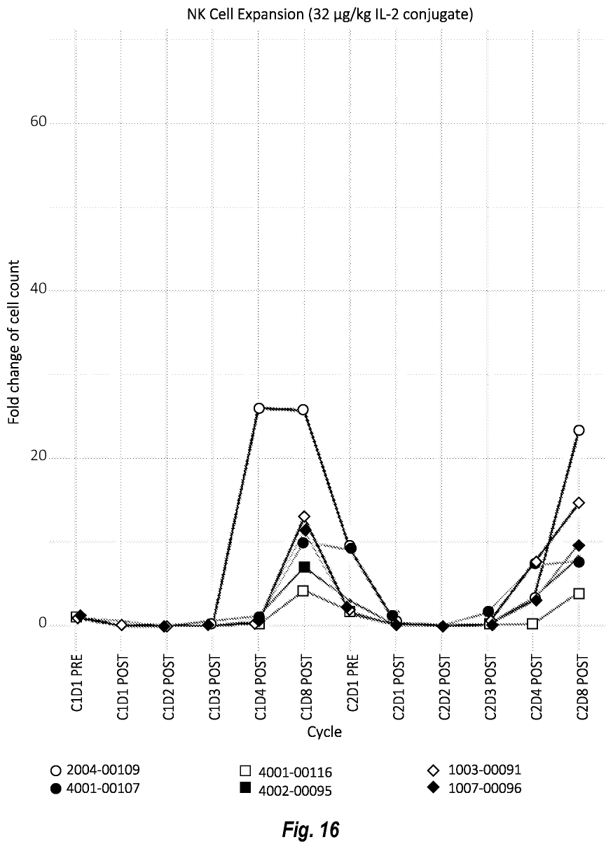

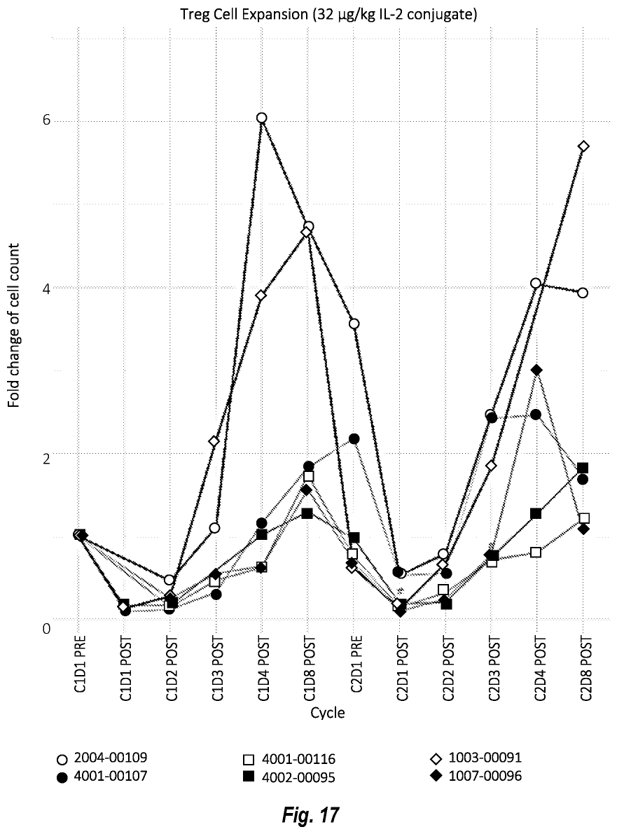

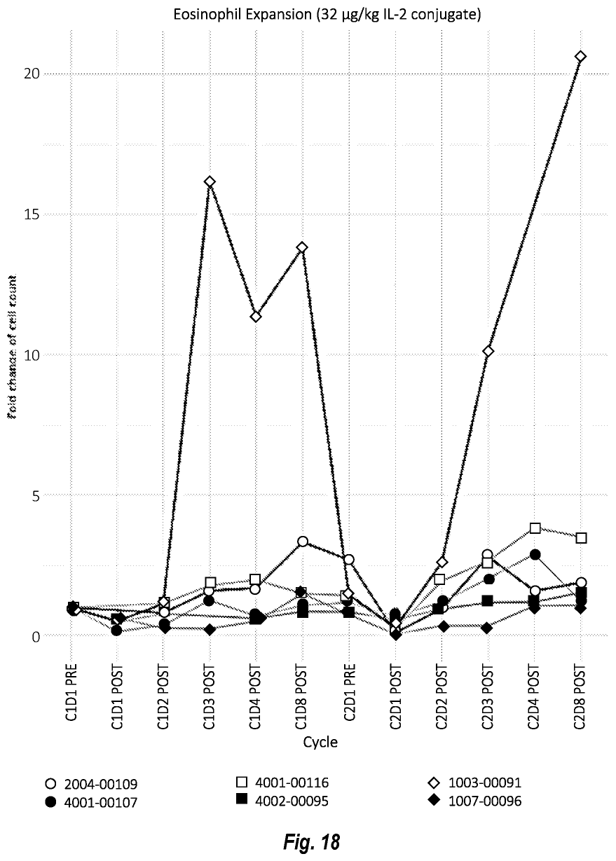

Peripheral CD8+ Teff cell counts of the subjects are shown in FIG. 15. Peripheral NK cell counts of the subjects are shown in FIG. 16. Peripheral CD4+T_{reg} counts of the subjects are shown in FIG. 17. Eosinophil counts of the subjects are shown in FIG. 18. Expansion of CD8+ cells and NK cells, with 32 μg/kg Q3W of the IL-2 conjugate+ cetuximab appeared higher than that of 24 μg/kg Q3W of the IL-2 conjugate+ cetuximab. Expansion of T_{reg} cells with 32 μg/kg Q3W of the IL-2 conjugate+ cetuximab appeared comparable to that with 24 μg/kg Q3W of the IL-2 conjugate+ cetuximab.

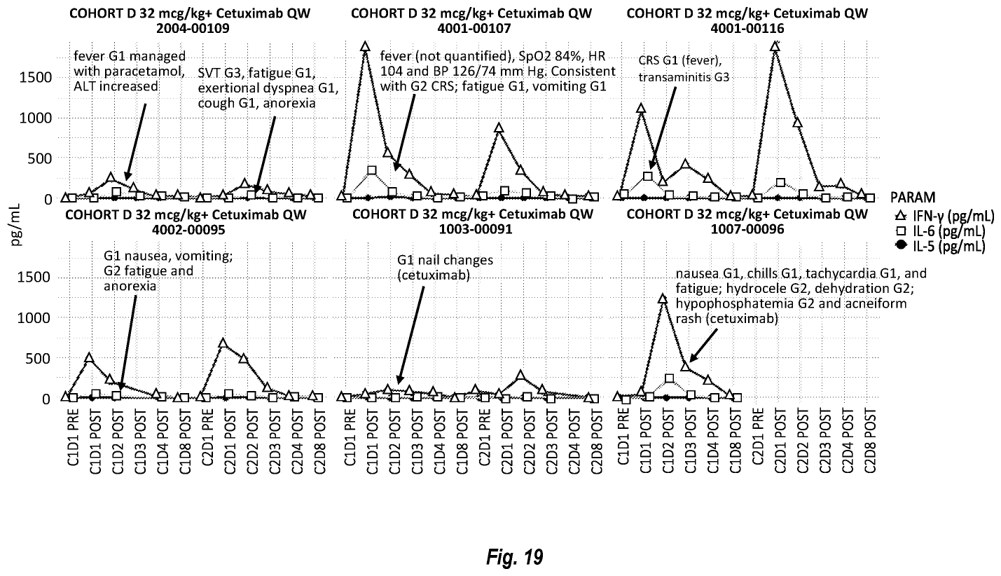

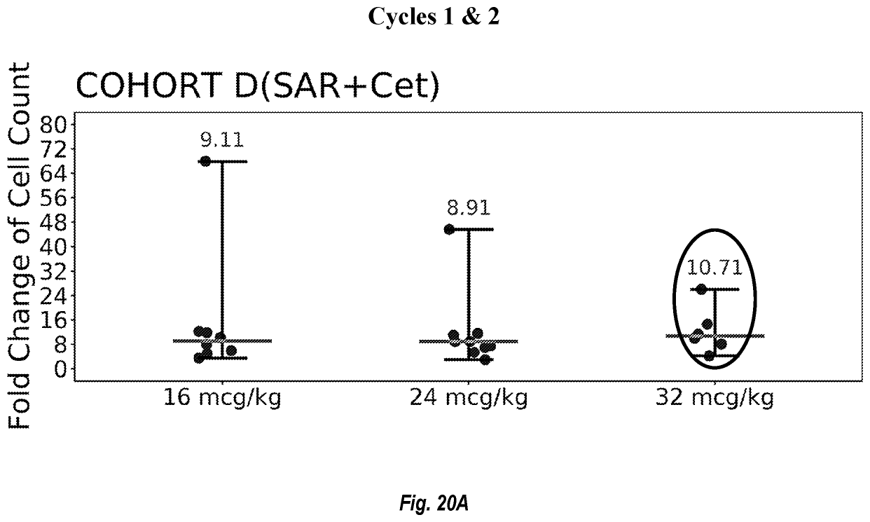

FIG. 19 shows shows serum levels of IFN-γ, IL-5, and IL-6 in the indicated subjects treated with 32 μg/kg of the IL-2 conjugate in combination with cetuximab at specified times following administration of the IL-2 conjugate. One subject developed an IL-6 level of about 250 μg/mL without symptoms suggestive of CRS. There were no elevations of IL-5.

Adverse events include Grade 1 fever, chills, tachycardia, nausea; Grade 2 fatigue, hydrocele, and dehydration; Grade 2 chills and hypophosphatemia (deemed related to cetuximab); Grade 1 nail changes deemed related to cetuximab (no other related TEAEs); Grade 1 nausea, vomiting; Grade 1 dry skin; Grade 1 CRS;Grade 2 fatigue and anorexia as well as oral ulcers (deemed unrelated to the IL-2 conjugate); Grade 2 CRS; Grade 3 supraventricular tachycardia; and Grade 3 transaminitis. Five (83.3%) of the six subjects experienced at least one TEAE. Two (33.3%) of the six subjects experienced at least one G3-4 related TEAE. There were no DLT's or drug discontinuations from TEAEs. Two subjects had dose reductions for Cycle 2, day 1, and both continue on treatment. Four (66.7%) of the six patients experienced SAEs and two (33.3%) of the six patients had three treatment related SAEs.

TEAEs are detailed for the six subjects in Table 6, and treatment-related adverse events for the six subjects are summarized in Table 7.

TABLE 6
Treatment Emergent Adverse Events: n = 6.

| System Organ Class | Grade 1 | Grade 2 | Grade 3 | Grade 4 | Grade 5 |
|---|---|---|---|---|---|
| Blood and Lymphatic | 1/6 (16.7%) | 0/6 (0%) | 0/6 (0%) | 0/6 (0%) | 0/6 (0%) |
| Disorders | | | | | |
| Gastrointestinal disorders | 5/6 (83.3%) | 0/6 (0%) | 0/6 (0%) | 0/6 (0%) | 0/6 (0%) |
| Skin and Subcutaneous | 6/6 (100.0%) | 0/6 (0%) | 0/6 (0%) | 0/6 (0%) | 0/6 (0%) |
| Tissue Disorders | | | | | |
| General Disorders and | 3/6 (50%) | 3/6 (50.0%) | 0/6 (0%) | 0/6 (0%) | 0/6 (0%) |
| Administration Conditions | | | | | |
| Metabolism and nutrition | 1/6 (16.7%) | 3/6 (50.0%) | 0/6 (0%) | 0/6 (0%) | 0/6 (0%) |
| disorders | | | | | |
| Respiratory and Thoracic | 2/6 (33.3%) | 0/6 (0%) | 1/6 (16.7%) | 0/6 (0%) | 1/6 (16.7%) |
| Disorders | | | | | |
| Eye Disorders | 2/6 (33.3%) | 0/6 (0%) | 0/6 (0%) | 0/6 (0%) | 0/6 (0%) |
| Cardiac Disorders | 1/6 (16.7%) | 0/6 (0%) | 1/6 (16.7%) | 0/6 (0%) | 0/6 (0%) |
| Immune System Disorders | 1/6 (16.7%) | 1/6 (16.7%) | 0/6 (0%) | 0/6 (0%) | 0/6 (0%) |
| Infections and infestations | 0/6 (0%) | 1/6 (16.7%) | 0/6 (0%) | 0/6 (0%) | 0/6 (0%) |
| Musculoskeletal Disorders | 0/6 (0%) | 0/6 (0%) | 1/6 (16.7%) | 0/6 (0%) | 0/6 (0%) |
| Nervous System Disorders | 1/6 (16.7%) | 0/6 (0%) | 1/6 (16.7%) | 0/6 (0%) | 0/6 (0%) |
| Reproductive Disorders | 1/6 (16.7%) | 0/6 (0%) | 0/6 (0%) | 0/6 (0%) | 0/6 (0%) |

TABLE 7
Treatment Related Adverse Events: n = 6.

| System Organ Class | Grade 1 | Grade 2 | Grade 3 | Grade 4 | Grade 5 |
|---|---|---|---|---|---|
| Gastrointestinal disorders | 4/6 (66.7%) | 0/6 (0%) | 0/6 (0%) | 0/6 (0%) | 0/6 (0%) |
| General Disorders and | 4/6 (66.7%) | 1/6 (16.7%) | 0/6 (0%) | 0/6 (0%) | 0/6 (0%) |
| Administration Conditions | | | | | |
| Metabolism and Nutrition | 1/6 (16.7%) | 2/6 (33.3%) | 0/6 (0%) | 0/6 (0%) | 0/6 (0%) |
| Disorders | | | | | |
| Skin and Subcutaneous | 1/6 16.7%) | 0/6 (0%) | 0/6 (0%) | 0/6 (0%) | 0/6 (0%) |
| Tissue Disorders | | | | | |
| Cardiac Disorders | 1/6 (16.7%) | 0/6 (0%) | 1/6 (16.7%) | 0/6 (0%) | 0/6 (0%) |
| Eye Disorders | 1/6 (16.7%) | 0/6 (0%) | 0/6 (0%) | 0/6 (0%) | 0/6 (0%) |
| Immune System Disorders | 1/6 (16.7%) | 1/6 (16.7%) | 0/6 (0%) | 0/6 (0%) | 0/6 (0%) |
| Respiratory Disorders | 1/6 (16.7%) | 0/6 (0%) | 0/6 (0%) | 0/6 (0%) | 0/6 (0%) |

Overall, for the 16 μg/kg and 24 μg/kg cohorts receiving cetuximab, sustained increases in CD8+ T cells (1.3 to 7.57-fold above baseline) and NK cells (3.6 to 45.4-fold above baseline) were observed. No vascular leak syndrome, QTc prolongation, cardiac or end organ toxicity, or IL-5 elevation was observed. There were no AEs associated with IL-6 increases. Effects on IL-6 levels were transient in nature. Also, CD4 and eosinophil levels did not exceed 150 cells/μL and 1,100 cells/μL, respectively. The most common AEs included pyrexia, nausea, flu-like symptoms, vomiting, chills, fatigue, and AST elevation. AEs generally resolved promptly with supportive care. Grade(G) 3/4 related AEs included ALT/AST elevation and decreased lymphocyte count (recovering by 48-72 hours, lymphocyte migration being mechanistically consistent with immune cell margination). G3/4 CRS was observed in two patients.

For the 32 μg/kg cohort receiving cetuximab, two subjects had low-grade CRS managed with supportive care. One of those subjects had G3 transaminitis, which recovered within four days. One subject had G3 supraventricular tachycardia. One subject had G4 decreased lymphocyte count. There were no DLT's. No cumulative toxicity, QTc prolongation, end organ toxicity, or meaningful IL-5 elevation was observed. Based on safety, NK cell expansion, T-cell expansion and cytokines, this dose appears tolerable.

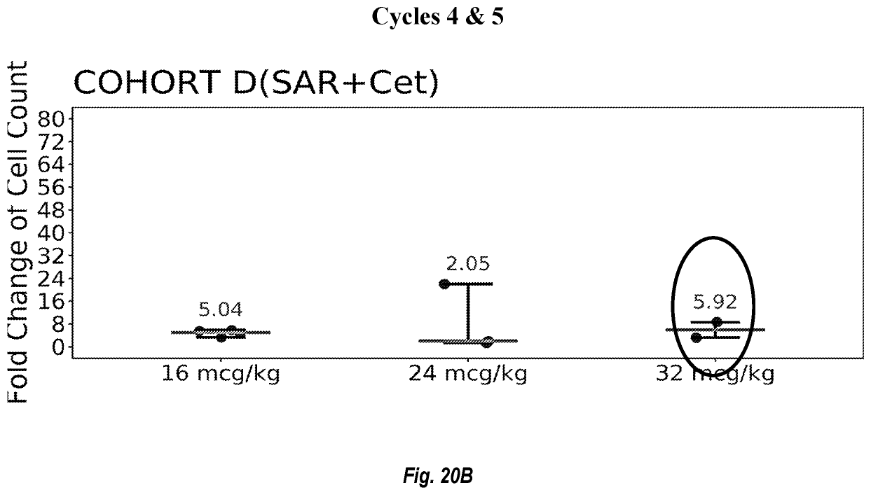

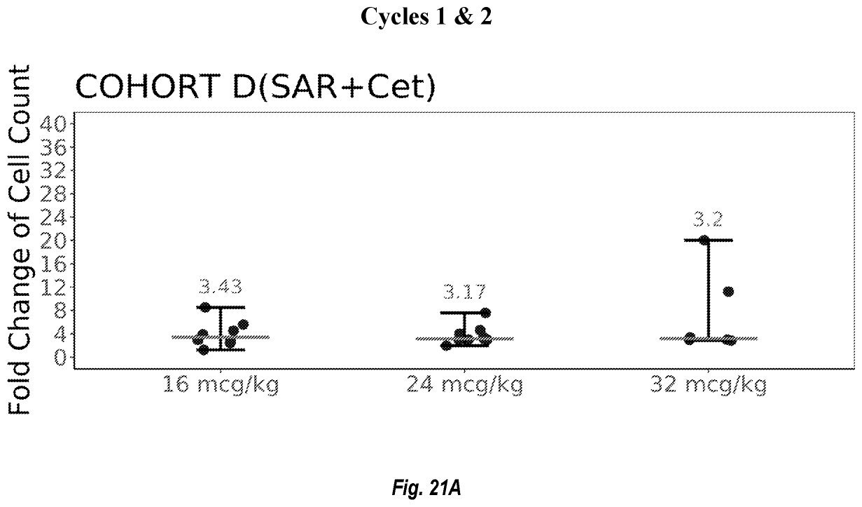

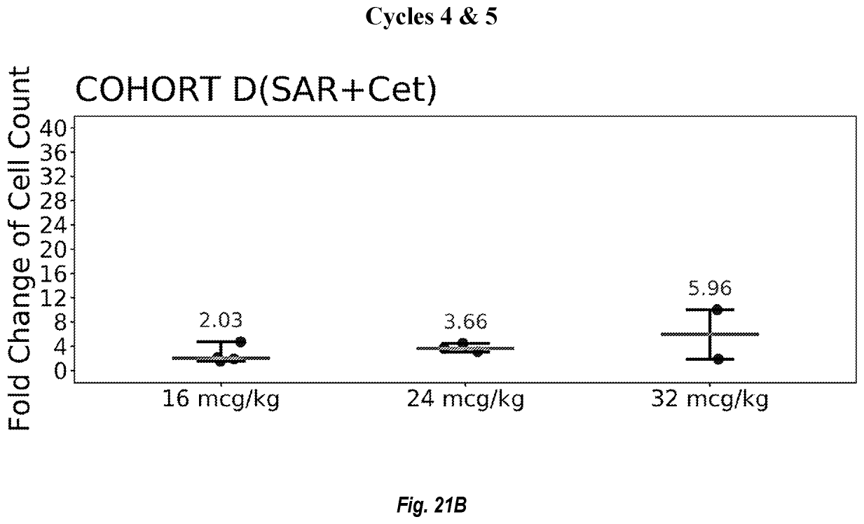

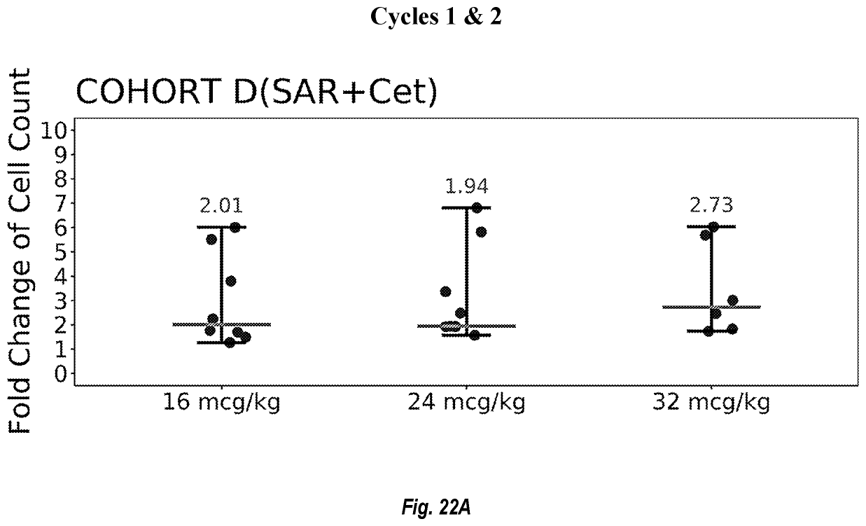

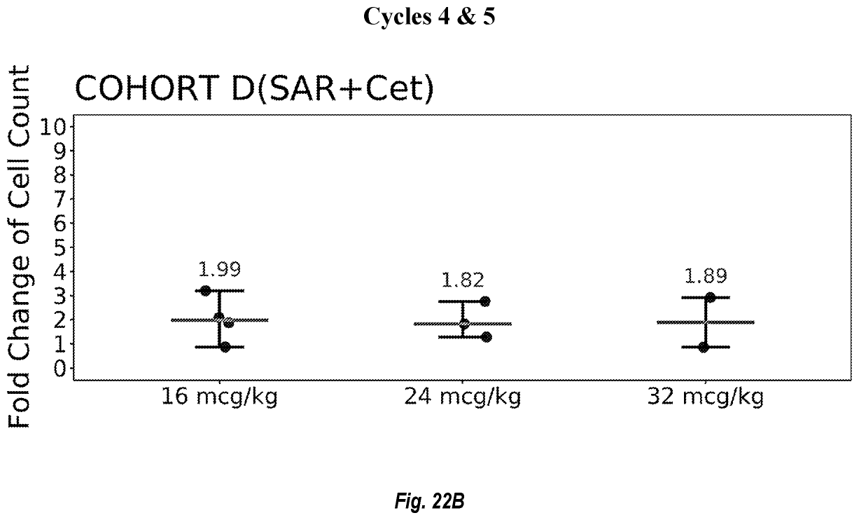

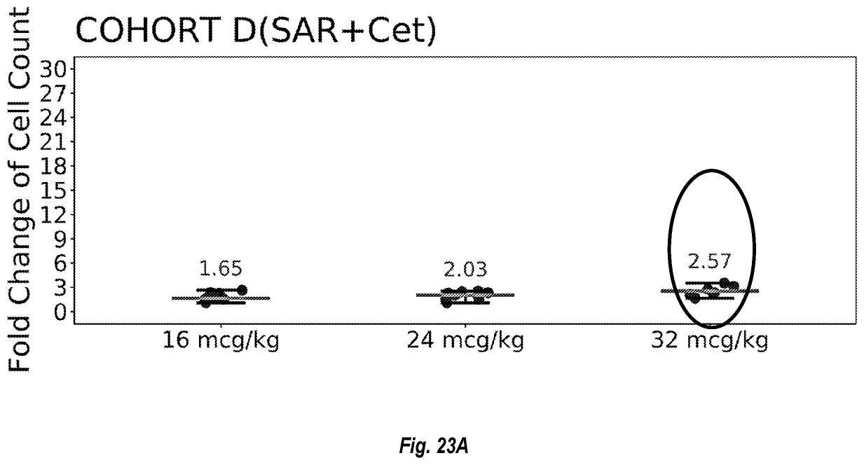

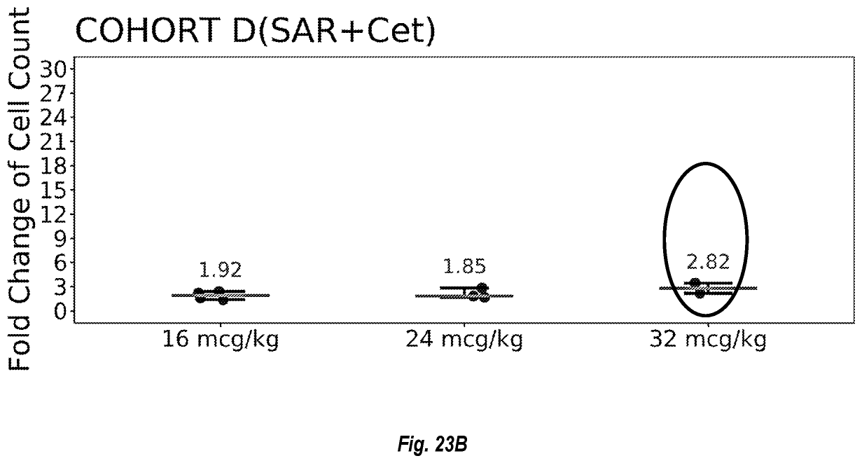

Peripheral NK cell counts of the subjects treated with 16, 24, or 32 μg/kg of the IL-2 conjugate in combination with cetuximab are shown in FIG. 20A (Cycles 1 and 2) and FIG. 20B (Cycles 4 and 5). Peripheral CD8+ Teff cell counts of the subjects treated with 16, 24, or 32 μg/kg of the IL-2 conjugate in combination with cetuximab are shown in FIG. 21A (Cycles 1 and 2) and FIG. 21B (Cycles 4 and 5). Peripheral CD4+T_{reg} counts of the subjects treated with 16, 24, or 32 μg/kg of the IL-2 conjugate in combination with cetuximab are shown in FIG. 22A (Cycles 1 and 2) and FIG. 22B (Cycles 4 and 5). Eosinophil counts of the subjects treated with 16, 24, or 32 μg/kg of the IL-2 conjugate in combination with cetuximab are shown in FIG. 23A (Cycles 1 and 2) and FIG. 23B (Cycles 4 and 5).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating head and neck squamous cell carcinoma (HNSCC) in a subject in need thereof, comprising administering to the subject (a) an IL-2 conjugate and (b) cetuximab, wherein:
the HNSCC is recurrent and/or metastatic HNSCC; and
the IL-2 conjugate comprises the amino acid sequence of SEQ ID NO: 1 wherein the amino acid at position P64 is replaced by the structure of Formula (I): 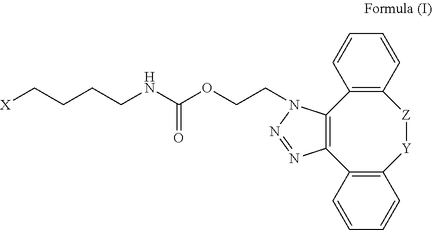
wherein:
Z is CH₂ and Y is 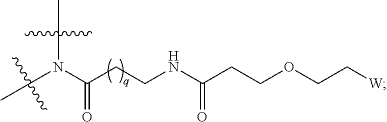
Y is CH₂ and Z is 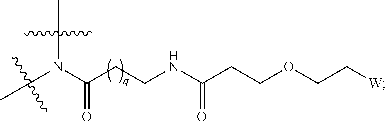
Z is CH₂ and Y is 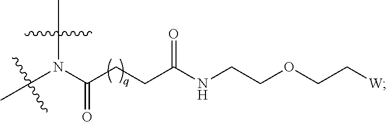
or
Y is CH₂ and Z is 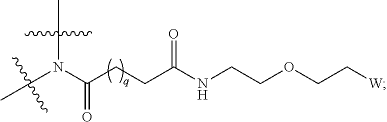
W is a PEG group having an average molecular weight of about 25 kDa-35 kDa;
q is 1, 2, or 3;
X is an L-amino acid having the structure: 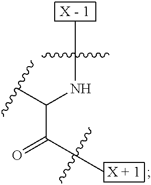
X−1 indicates the point of attachment to the preceding amino acid residue; and
X+1 indicates the point of attachment to the following amino acid residue.

2. A method of treating head and neck squamous cell carcinoma (HNSCC) in a subject in need thereof, comprising:
selecting a subject having HNSCC, wherein the subject is selected at least in part on the basis of the subject having recurrent and/or metastatic HNSCC; and
administering to the subject (a) an IL-2 conjugate, and (b) cetuximab, wherein:
the IL-2 conjugate comprises the amino acid sequence of SEQ ID NO: 1 wherein the amino acid at position P64 is replaced by the structure of Formula (I): 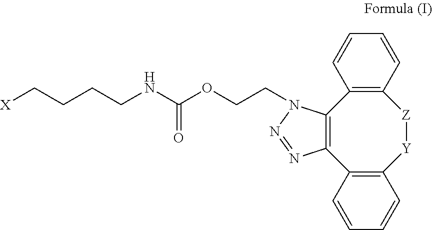
wherein:
Z is CH₂ and Y is 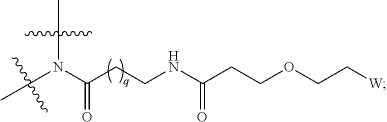
Y is CH₂ and Z is 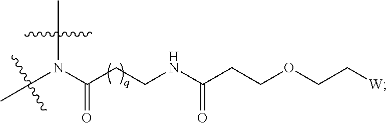
Z is CH₂ and Y is 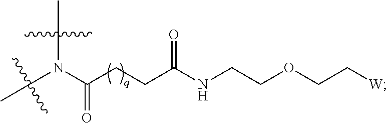
or
Y is CH₂ and Z is 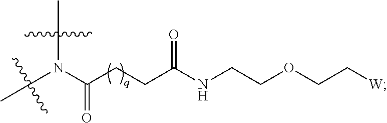
W is a PEG group having an average molecular weight of about 25 kDa-35 kDa;
q is 1, 2, or 3;
X is an L-amino acid having the structure: 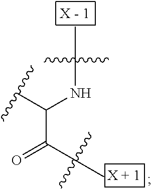 [SMILES: *NC(*)C(*)=O]
X−1 indicates the point of attachment to the preceding amino acid residue; and
X+1 indicates the point of attachment to the following amino acid residue.

3. A method of treating head and neck squamous cell carcinoma (HNSCC) in a subject in need thereof, comprising administering to the subject (a) from 8 μg/kg to 32 μg/kg IL-2 as an IL-2 conjugate and (b) cetuximab, wherein:
the IL-2 conjugate comprises the amino acid sequence of SEQ ID NO: 1 wherein the amino acid at position P64 is replaced by the structure of Formula (I): 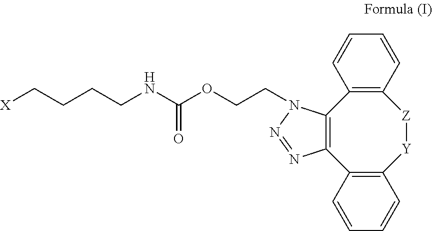
wherein:
Z is CH₂ and Y is 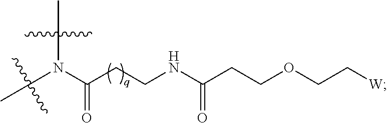
Y is CH₂ and Z is 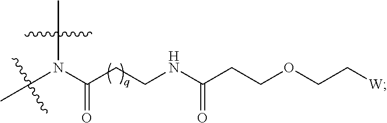
Z is CH₂ and Y is 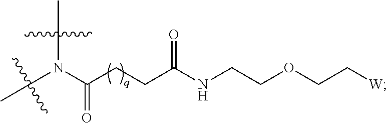
or
Y is CH₂ and Z is 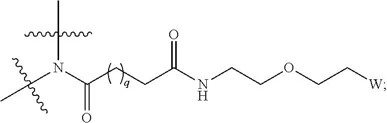
W is a PEG group having an average molecular weight of about 25 kDa-35 kDa;
q is 1, 2, or 3;
X is an L-amino acid having the structure: 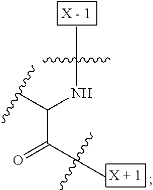 [SMILES: *NC(*)C(*)=O]
X−1 indicates the point of attachment to the preceding amino acid residue; and
X+1 indicates the point of attachment to the following amino acid residue.

4. The method of claim 1, wherein the subject was not previously treated with cetuximab.

5. The method of claim 1, wherein the subject has platinum-refractory HNSCC.

6. The method of claim 1, wherein the subject was previously treated for HNSCC and the previous treatment for HNSCC comprised failure of no more than two regimens.

7. The method of claim 1, wherein the subject has platinum-refractory HNSCC and the subject's previous treatment for HNSCC comprised failure of one or two regimens.

8. (canceled)

9. The method of claim 1, comprising administering to the subject about 8 μg/kg to 32 μg/kg IL-2 as the IL-2 conjugate.

10. (canceled)

11. The method of claim 1, comprising administering to the subject about 16 μg/kg IL-2 as the IL-2 conjugate.

12. The method of claim 1, comprising administering to the subject about 24 μg/kg IL-2 as the IL-2 conjugate.

13. The method of claim 1, comprising administering to the subject about 32 μg/kg IL-2 as the IL-2 conjugate.

14. The method of claim 1, wherein in the TL-2 conjugate the PEG group has an average molecular weight of about 30 kDa.

15. (canceled)

16. (canceled)

17. The method of claim 1, wherein in the IL-2 conjugate Z is CH₂ and Y is 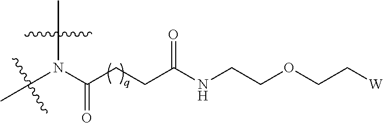
or Y is CH₂ and Z is

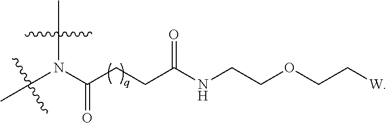

18. (canceled)

19. The method of claim 1, wherein the structure of Formula (I) has the structure of Formula (IV) or Formula (V), or is a mixture of Formula (IV) and Formula (V): 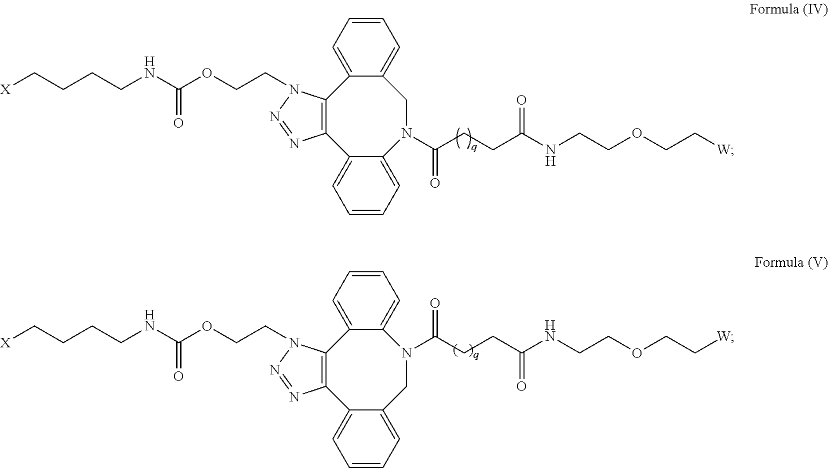
wherein:
W is a PEG group having an average molecular weight of about 25 kDa-35 kDa;
q is 1, 2, or 3;
X is an L-amino acid having the structure: 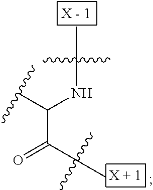 [SMILES: *NC(*)C(*)=O]
X−1 indicates the point of attachment to the preceding amino acid residue; and
X+1 indicates the point of attachment to the following amino acid residue.

20. The method of claim 1, wherein the structure of Formula (I) has the structure of Formula (XII) or Formula (XIII), or is a mixture of Formula (XII) and Formula (XIII): 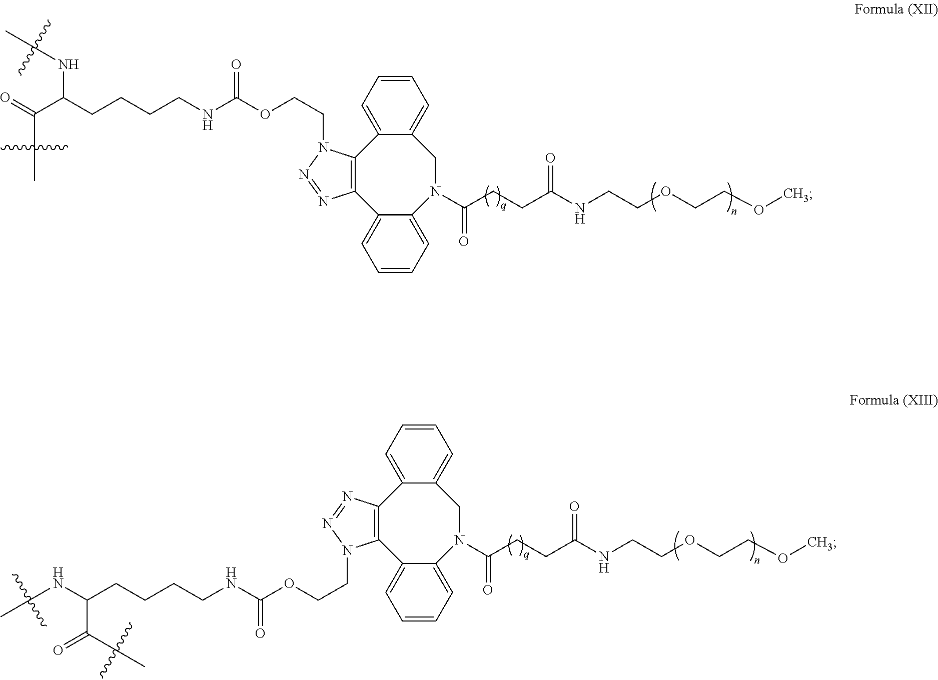
wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

21. The method of claim 1, wherein q is 1.

22. (canceled)

23. (canceled)

24. The method of claim 1, wherein the average molecular weight is a number average molecular weight or a weight average molecular weight.

25. (canceled)

26. The method of claim 1, wherein the IL-2 conjugate is administered to the subject about once every two weeks, about once every three weeks, or about once every 4 weeks: optionally wherein cetuximab is administered to the subject about once every two weeks, about once every three weeks, or about once every 4 weeks.

27. (canceled)

28. The method of claim 1, wherein the IL-2 conjugate is a pharmaceutically acceptable salt, solvate, or hydrate.

29. The method of claim 1, wherein the initial dose of cetuximab is administered at a dose of about 400 mg/m², and subsequent doses of cetuximab are administered at a dose of about 250 mg/m².

30. (canceled)

31. The method of claim 1, wherein the IL-2 conjugate and cetuximab are administered separately or sequentially, or the IL-2 conjugate is administered after cetuximab.

32. (canceled)

33. (canceled)

34. The method of claim 1, wherein the IL-2 conjugate is administered to the subject by subcutaneous administration, optionally wherein cetuximab is administered to the subject by subcutaneous administration.

35. (canceled)

36. The method of claim 1, wherein the IL-2 conjugate is administered to the subject by intravenous administration, optionally wherein cetuximab is administered to the subject by intravenous administration.

37. (canceled)

38. The method of claim 1, further comprising administering acetaminophen, diphenhydramine, and/or ondansetron to the subject.

39. (canceled)

40. (canceled)

41. The method of claim 38, wherein the acetaminophen, diphenhydramine, and/or ondansetron is administered to the subject before administering the IL-2 conjugate and/or cetuximab.

42. (canceled)

43. (canceled)

44. (canceled)


SEQUENCE LISTING (2 sequences)

SEQ ID NO: 1 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, synthetic construct
  MOD_RES 64: note = Xaa is [AzK_L1_PEG30kD] (N6-((2-azidoethoxy)-carbonyl)-L-lysine stably-conjugated to PEG via DBCO-mediated click chemistry)
PTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT